(19) United States
(12) Patent Application Publication
(10) Pub. No.: US 2022/0401586 A1
(43) Pub. Date: Dec. 22, 2022

(54) CARBON QUANTUM DOTS AND USES THEREOF

(71) Applicant: CNANO MEDICINE CORP., Beijing (CN)

(72) Inventors: Louzhen FAN, Beijing (CN); Shuhua LI, Beijing (CN); Wen SU, Beijing (CN); Hao WU, Beijing (CN)

(73) Assignee: CNANO MEDICINE CORP., Beijing (CN)

(21) Appl. No.: 17/773,759

(22) PCT Filed: Nov. 4, 2019
(86) PCT No.: PCT/CN2019/115263

§ 371 Date: May 2, 2022

(51) Int. Cl. A61K 49/00; C09K 11/65

(52) U.S. Cl. CPC A61K 49/0067; C09K 11/65; A61K 2121/00; A61K 2123/00

(57) ABSTRACT

Polycyclic compounds that are aromatic or partially aromatic, and are substituted with one or more alkyl groups having an amino group and a carboxylic acid group, and includes carbon quantum dots (CQDs) that contain these compounds. The compounds and CQDs have a selective affinity for cells that express LAT1 and for tumor cells, and can internalize within such cells. The compounds and CQDs are useful for imaging of such cells and for delivering cargo compounds to and into cells that express LAT1 and tumor cells. Methods for making and using these compounds and CQDs for bioimaging and targeting certain tissues, including tumors, are disclosed.

### FIELD OF THE INVENTION

The field of this invention is compounds, compositions and methods related to imaging of tumors and delivery of therapeutic agents to tumors, as well as methods to make the novel compounds. The methods and compositions involve polycyclic aromatic structures that often function as carbon quantum dots (CQDs).

### BACKGROUND OF THE INVENTION

Cancer is one of the most devastating diseases, with more than 14 million new cases every year. The incidence of cancer is projected to continue to rise, with an estimated 21.7 million new cases annually by 2030. In current clinical practices, cancer patients are typically managed through a combination of surgical resection, if applicable, in conjunction with chemotherapy and radiotherapy. With tremendous advances in surgery and medicine over the past few decades, this combination has significantly increased survival times and quality of life for patients. However, further improvement of these approaches has been hindered by the inability to selectively image and deliver therapeutics to tumors. This challenge limits our ability to achieve maximum surgical resection or to deliver chemotherapeutic agents and radiotherapy without induction of significant side effects.¹⁻⁷

To enhance tumor-specific imaging and drug delivery, recent efforts have been focused on engineering imaging or therapeutic agents through conjugation of ligands that recognize “receptor” molecules expressed on cancer cells or in the tumor microenvironment. Accumulating evidence suggests that this approach has several major limitations. First, receptor molecules that are exclusively expressed on the surface of cancer cells are rare-most are also present in normal tissues. As a result, the accumulation of cargo agents in normal tissues cannot be avoided. Second, cancer represents a group of highly heterogeneous diseases. Cancers of different tissue origin or intra-tumor location often have distinct genotypes and phenotypes. Consequently, they have not been successfully targeted using a single ligand. Due to these limitations, a strategy that enables selective imaging and drug delivery to tumors, regardless of their origin and location, has not yet been achieved.

One promising approach to tumor-specific imaging and drug delivery leverages specific carrier transporters that are differentially upregulated in cancer cells. Examples of such transporters include large neutral amino acid transporter 1 (LAT1), alanine, serine, cysteine-preferring transporter 2 (ASCT2), and glucose transporters.⁸⁻¹¹ Of these, LAT1, which mediates transport of large-neutral amino acids, is particularly intriguing. LAT1 has been shown to be highly expressed in a wide variety of tumors. In stark contrast, based on the expression of mRNA, the distribution of LAT1 in normal tissues is restricted to only a few organs, including the placenta, BBB, spleen, testis, and colon.¹⁰ LAT1 has been previously targeted for cancer chemotherapy using LAT1 inhibitors, such as 2-aminobicyclo-(2,2,1)-heptane-2-carboxylic acid (BCH).¹² Unfortunately, BCH-mediated chemotherapy has largely failed because of the lack of potency and specificity. BCH inhibits all four LATs, including LAT1, LAT2, LAT3 and LAT4, each of which has distinct physiological functions. Treatment with the required high doses of BCH often induces significant toxicity.¹² LAT1 has also been targeted to enhance the delivery of chemotherapy drugs to tumors. It was shown that conjugation of aspartate enabled LAT1-mediated delivery, improving the accumulation of doxorubicin (DOX) in tumors by 3-6-fold.¹³

Due to their excellent biocompatibility, optical properties, drug loading capacity, and low toxicity, CQDs have recently emerged as a promising class of imaging agents and drug carriers for various biomedical applications. However, most of the previously reported CQDs produce non-selective interactions with both tumor cells and non-tumor cells, and do not achieve a level of tumor specificity to make them useful for selectively targeting tumor cells.^{14,15}

### SUMMARY OF THE INVENTION

The present invention provides large amino acid-mimicking compounds, and large amino acid-mimicking carbon quantum dots (LAAM CQDs) comprising these compounds, which are useful for selective imaging of tissues that express LAT1 and for delivery of drugs and/or detectable labels to such tissues. Because many cancer cells express LAT1 at levels much higher than most human tissues, the compounds and CQDs can be used for imaging of tumors and for drug delivery to and into tumor cells, regardless of the tumor's origin and location, with little or no delivery into cells of normal tissues.

The LAAM compounds of the invention are polycyclic compounds having a large aromatic or partially aromatic core substituted with alpha-amino carboxylic acid moieties. The core of the compounds is a polycyclic array of 8 or more 6-membered rings, each of which is aromatic or partially aromatic or partially unsaturated. The polycyclic core is substituted with one or more, typically two or more, and in many embodiments three or four or more alkyl groups, each of which is substituted with a carboxylic acid and an amino group on the same carbon as the carboxylic acid, i.e., an alpha-amino carboxylic acid moiety. The amino group can be —NH₂ or a substituted version thereof. The alkyl group can also have one or more additional substituents.

LAAM compounds of the invention are able to assemble into aggregates that function as carbon quantum dots (LAAM CQDs). LAAM CQDs have an α-amino carboxylic acid moiety that is believed to trigger multivalent interaction and thus tight binding with LAT1. Without being bound by theory, the high and selective affinity these compounds and LAAM CQDs exhibit toward LAT1 are believed to account for their ability to selectively target tumor cells for both imaging of tumors and for delivery of cargo compounds such as therapeutic and cytotoxic agents to tumor cells. Some of the CQDs provided by the invention including LAAM TC-CQDs described herein, exhibit capacity for near-infrared (NIR) fluorescence (FL) and photoacoustic (PA) imaging, which allow imaging of tumors. Because they are selectively internalized into cancerous cell or tissue, such as by endocytosis through or transported by LAT1 into cells that express LAT1, the compounds and CQDs of the invention can also function as carriers for delivery of chemotherapeutic drugs to tumors. The LAAM CQDs disclosed herein, including LAAM CQDs synthesized using 1,4,5,8-tetraminoanthraquinone (TAAQ) and citric acid (CA), provide new compositions and methods for selectively imaging tumors and certain other types of cells, and for selectively delivering therapeutic or labeling agents to these cells and tumors.

In one aspect the invention provides carbon quantum dots that have useful fluorescent properties and are soluble in water and biocompatible. The carbon quantum dots of the invention can form organic (carbon-based) particles about 0.2 to 10 nm in size, which are typically at least about 20% or 40%, and preferably at least about 50% carbon by weight. Their fluorescent properties, water solubility, and biocompatibility make them useful in vivo, and the LAAM CQDs of the invention that selectively bind to LAT1 are especially useful in methods described herein for imaging tumors.

In another aspect, the invention provides solvolysis products formed by methods described herein that are effectively internalized into cells, e.g., via endocytosis or transported by LAT1, and are selectively taken up by tumor cells, especially in tumors that express LAT1. These solvolysis products comprise a polycyclic aromatic or partially aromatic core ring system, and can be isolated and used as particles about 0.2 to 10 nm in size that act as carbon quantum dots. Use of solvolysis for preparing carbon quantum dots is known in the art. These compounds and particles made by the methods herein can be used for imaging tumors or as carriers to deliver a payload such as a label or a therapeutic agent to the surface of a tumor cell that expresses LAT1, or into the interior of such cells. In some embodiments, the solvolysis products form particles that comprise one or more of the LAAM compounds disclosed herein, which behave as carbon quantum dots and are thus useful for imaging or targeting of tumors that express the LAT1 transporter.

In another aspect, the invention provides methods of making the compounds and LAAM CQDs described above.

In another aspect, the invention provides methods of using the compounds and CQDs containing them for imaging tissues that express LAT1, and especially for imaging tumors that express LAT1 in vivo.

In another aspect, the invention provides conjugates comprising the compounds and CQDs described above, and methods of using these conjugates to selectively deliver imaging agents, therapeutic agents, and cytotoxins to cells that express LAT1, including tumor cells.

In still another aspect, the present disclosure provides for a pharmaceutical composition comprising a compound described above admixed with at least one pharmaceutically acceptable carrier or excipient.

In yet another aspect, the present disclosure provides for a method of using the LAAM compounds and LAAM CQDs disclosed herein to image a tumor or a tissue that expresses LAT1.

In yet another aspect, the present disclosure provides for a use of the LAAM compounds and LAAM CQDs disclosed above for the manufacture of a medicament.

In yet another aspect, the present disclosure provides for a conjugate comprising an LAAM compound or LAAM CQDs of the invention linked covalently or non-covalently to a cargo compound to be delivered to or into a cell that expresses LAT1. Since many tumors express LAT1, these conjugates are useful to deliver labeling or chemotherapeutic agents to tumor cells selectively, with little or no delivery into normal cells.

In yet another aspect, the invention provides methods of using the conjugates of the invention to image and/or to treat tumors that express LAT1.

Other aspects and advantages of the products, compositions and methods of the invention will be apparent to the skilled person in view of the examples and further description that follow.

### BRIEF DESCRIPTION OF THE DRAWINGS

FIG. 1a is a Schematic diagram and hypothetical steps (dashed line) of LAAM TC-CQDs synthesis.

FIG. 1b shows TEM and HRTEM image (insert) of LAAM TC-CQDs.

FIG. 1c shows UV-vis absorption spectrum of LAAM TC-CQDs and appearance of LAAM TC-CQDs under daylight (insert).

FIG. 1d shows fluorescence emission spectra and appearance of LAAM TC-CQDs under a UV light at 365 nm (insert).

FIG. 1e shows the photoacoustic (PA) signal intensity and imaging (insert) of LAAM TC-CQDs at a concentration of 2 μg/mL.

FIG. 2a shows laser confocal scanning microscopy (LCSM) images of representative cells, including HeLa cells, and non-cancerous CCC-ESF-1 cells after incubation with LAAM TC-CQDs. Scale bar: 100 m.

FIG. 2b: Quantification of cellular uptake in cancerous and non-cancerous cells by flow cytometry.

FIG. 2c provides near IR fluorescence (NIR FL) images of a representative tumor-bearing mouse after receiving intravenous injection of LAAM TC-CQDs at the indicated time points.

FIG. 2d shows a 3D reconstruction of LAAM TC-CQDs distribution in the mouse of FIG. 2c, 8 h after injection.

FIG. 2e shows ex vivo NIR FL imaging of the indicated organs and tumor of the mouse of 2c, 8 h after injection of LAAM TC-CQDs.

FIG. 2f provides cross-sectional photoacoustic (PA) imaging of the indicated tumor (circled) in a representative tumor-bearing mouse receiving intravenous injection of LAAM TC-CQDs at the indicated time points.

FIG. 3a provides LCSM images of HeLa cells pretreated with Leu, Phe, Gly or 2-aminobicyclo-(2,2,1)-heptane-2-carboxylic acid (BCH, a known inhibitor of LAT1), followed by incubation with LAAM TC-CQDs. Scale bar: 25 m.

FIGS. 3b-d show down-regulation of LAT1 expression by CRISPR/Cas9 reduced cellular uptake of LAAM TC-CQDs (bottom panel). Red arrows in b indicate the sgRNA targeting sequences. Successful targeting of LAT1 was confirmed by Sanger sequencing (b, upper panel) and WB test (c). LCSM images of cells with down-regulation of LAT1 were shown in d.

FIG. 3e shows correlation of LAT1 expression with cellular uptake of LAAM TC-CQDs.

FIG. 4a: LCSM images of HeLa and CCC-ESF-1 cells after incubation with TPTC, LAAM TC-CQDs or TPTC/LAAM TC-CQDs.

FIG. 4b: Viability of HeLa and CCC-ESF-1 cells after treatment with LAAM TC-CQDs TPTC or TPTC/LAAM TC-CQDs.

FIG. 4c shows changes in serum concentration of TPTC, when delivered in form of free drug or with TPTC/LAAM TC-CQDs, over time. Data are expressed as percentage of total injected dose (% ID).

FIG. 4d shows biodistribution of TPTC and TPTC/LAAM TC-CQDs in indicated organs.

FIG. 4e shows representative images of tumor-bearing mice receiving the indicated treatments.

FIG. 4f: Representative images of tumors obtained from mice receiving the indicated treatments at day 15 after treatment.

FIG. 4g: Changes of tumor volume with time in mice receiving the indicated treatments (n=5).

FIG. 5a: molecular structures of selected compounds that form LAAM TC-CQDs, including NH₂— and COOH-null LAAM TC-CQDs, 2,6-CQDs, 1,4-CQDs and 1,5-CQDs and Phe-CQDs.

FIG. 5b: Images showing cell uptake/penetration by the LAAM TC-CQDs shown in FIG. 5a.

FIG. 5c, Flow cytometry quantification of FL intensities in HeLa or CCC-ESF-1 cells after treatment with the indicated CQDs.

FIG. 6. The size distribution of TC-CQDs.

FIG. 7. Raman spectrum of the TC-CQDs.

FIG. 8(a) AFM image of the TC-CQDs on a Si substrate.

FIG. 8(b) Height profile along the lines in FIG. 9(a).

FIG. 9. XRD pattern of the LAAM TC-CQDs.

FIG. 10. XPS survey spectrum of the LAAM TC-CQDs.

FIG. 11. C1s spectra of LAAM TC-CQDs.

FIG. 12. N1s spectra of LAAM TC-CQDs.

FIG. 13. FT-IR spectrum of LAAM TC-CQDs.

FIG. 14. ¹³C-NMR spectrum of LAAM TC-CQDs.

FIG. 15. Fluorescence emission spectrum of TC-CQDs aqueous solution with various excitation wavelength from 560 to 660 nm.

FIG. 16. The main geometric parameters (A) for the optimized ground (a) and excited (b) structures of one FL unit of LAAM TC-CQDs. The optimized electron delocalization molecular orbital (MO) diagrams of TC-CQDs (c, d) and its band positions (e) obtained from theoretical calculation with density functional theory calculations (B3LYP/6-31G(d,p)).

FIG. 17(a) The photothermal curves of TC-CQDs at various concentrations (0-10 g/mL) under 650 nm laser irradiation (0.5 W/cm²) recorded every 30 s. The inset are IR thermal images of TC-CQDs (0-10 μg/mL) after 650 nm laser irradiation (0.5 W/cm²) for 5 min.

FIG. 17(b) The temperature change of LAAM TC-CQDs (10 μg/mL) for five laser on/off cycles (650 nm laser irradiation, 0.5 W/cm²).

FIG. 17(c) Photothermal effect of the TC-CQDs aqueous solution when illuminated with 650 nm laser (0.5 W/cm²). The laser was shut off after irradiation for 5 min.

FIG. 17(d) Plot of cooling time versus negative natural logarithm of the temperature driving force obtained from the cooling stage as shown (c). The time constant for heat transfer of the system was determined to be τs=83.86 s.

FIG. 18. Photoacoustic (PA) signal intensities of LAAM TC-CQDs at concentrations ranging from 0 to 10 μg/mL.

FIG. 19. PA signal intensity and imaging of LAAM TC-CQDs at concentrations ranging from 2 to 10 μg/mL.

FIG. 20. Characterization of side population cells sorted from MDA-MB-231 cells. (a) was from the cells stained with isotype controls; (b) was from the cells stained with antihuman CD44-FITC and CD24-PE antibodies. CSCs cells were analyzed with flow cytometry.

FIG. 21. LCSM images of different types of cancer cells co-incubated with LAAM TC-CQDs. The scale bar is 100 m.

FIG. 22. LCSM images of different types of normal cells co-incubated with LAAM TC-CQDs. The scale bar is 100 m.

FIG. 23. Uptake rates of LAAM TC-CQDs in 20 types of cancer cells obtained by flow cytometric profiles.

FIG. 24. Uptake rates of LAAM TC-CQDs in 18 types of normal cells obtained by flow cytometric profiles.

FIG. 25. LCSM images of HeLa cells treated with LAAM TC-CQDs for different time (1-10 h). The scale bar is 25 m.

FIG. 26. LCSM images of CCC-ESF-1 cells treated with LAAM TC-CQDs for different times (1-48 h). The scale bar is 25 m.

FIG. 27. Semiquantitative biodistribution of LAAM TC-CQDs in tumor-bearing mice determined by the averaged FL intensity of major organs and tumors.

FIG. 28. The PA signal intensities at the tumor area for different times.

FIG. 29. NIR FL images of mice with subcutaneous tumor xenografts derived from A549 (a), PANC-1 (b), MCF-7 (c) and MD-MBA-231 (d) cells following intravenous injection of LAAM TC-CQDs during 10 h.

FIG. 30. Ex vivo NIR FL imaging of major organs (heart, liver, spleen, lung and kidney) and tumors from nude mice with subcutaneous tumor xenografts derived from A549 (a), PANC-1 (b), MCF-7 (c) and MD-MBA-231 (d) cells after the injection of LAAM TC-CQDs at 8 h post-injection.

FIG. 31. NIR FL images of nude mice with hepatic (A549) tumor (left side) and ovarian (HeLa) tumor (right side) following intravenous injection of LAAM TC-CQDs during 10 h.

FIG. 32. Ex vivo NIR FL imaging of major organs (heart, liver, spleen, lung and kidney) and tumors from nude mice with hepatic (A549) tumor and ovarian (HeLa) tumor (right) after the injection of LAAM TC-CQDs at 8 h post-injection.

FIG. 33. LCSM images of HeLa and CCC-ESF-1 cells treated with G-CQDs, Y-CQDs, B-CQDs and B,S-CQDs, respectively. The scale bar is 100 m. Note: G-CQDs refer to green fluorescent CQDs synthesized by pyrolysis of citric acid; Y-CQDs refer to yellow fluorescent CQDs synthesized by electrolysis of graphite in alkaline condition and reduction of the products with hydrazine at room temperature; B-CQDs refer to boron (B) doped CQDs synthesized by electrolysis of graphite in borax aqueous solution; B,S-CQDs refer to boron (B) and sulfur (S) co-doped CQDs synthesized by electrolyzing graphite rods in sodium p-toluenesulfonate (TsONa) acetonitrile solution.

FIG. 34. The uptake rates of G-CQDs, Y-CQDs, B-CQDs and B,S-CQDs in HeLa and CCC-ESF-1 cells.

FIG. 35. (a) Three-dimensional reconstruction of G-CQDs distribution in the tumor 8 h after injection.

FIG. 36. (a) Ex vivo NIR FL imaging of major organs (heart, liver, spleen, lung and kidney) and tumors after the injection of G-CQDs at 8 h post-injection. (b) Semiquantitative biodistribution of G-CQDs in tumor-bearing mice determined by the averaged FL intensity of major organs and tumors after the injection of G-CQDs at 8 h post-injection.

FIG. 37. NIR FL images of nude mice with hepatic (A549) tumor left and ovarial (HeLa) tumor right following intravenous injection of FA-G-CQDs during 10 h.

FIG. 38. Ex vivo NIR FL imaging of major organs (heart, liver, spleen, lung and kidney) and tumors after the injection of FA-G-CQDs with surface conjugation of folic acid (FA) (FA-G-CQDs) at 8 h post-injection.

FIG. 39. The uptake rates of LAAM TC-CQDs (control), LAAM TC-CQDs pretreated with Leu, Phe, Gly or BCH in HeLa, A549, PANC-1, MCF-7, MDA-MB-231 and CSCs cells.

FIG. 40. The uptake rates of LAAM TC-CQDs in wild type HeLa cells, sgLAT1-1 and sgLAT1-2 HeLa cells, respectively.

FIG. 41. The relative LAT1 expressions in different types of cells were measured by WB tests. For cancer cells, from left to right is HeLa, A549, PANC-1, MCF-7, MDA-MB-231, MKN-45, A498, HepG2, HTB-9, EC109, PC-3, SK-MEL-1, NCI-H1975, T84, CAL-27, SH-SY5Y, Hep-2, H460, Jurkat, Breast CSCs cells; For normal cells, from left to right is CCC-ESF-1, HUVEC, CCD-1095SK, HL-7702, RWPE-1, HaCaT, Hs578Bst, MCF 10A, BEAS-2B, MRC-5, WISH, HKC, 1301, HLF-a, CCC-HEK-1, CCC-HPE-2, CCC-HIE-2, HBMSCs cells.

FIG. 42. UV-vis absorption spectra of (a) LAAM TC-CQDs, TPTC and TPTC/LAAM TC-CQDs, (b) TC-CQDs, DOX and DOX/LAAM TC-CQDs, and (c) LAAM TC-CQDs, HCPT and HCPT/LAAM TC-CQDs aqueous solution.

FIG. 43. The FL emission spectrum of TPTC.

FIG. 44. Mean FL intensities of TPTC and TPTC/LAAM TC-CQDs in the nuclei were calculated using Image-Pro Plus 6.0 software.

FIG. 45. Cell viabilities of HeLa, A549, PANC-1, MCF-7 and MDA-MB-231 cells after treatment with LAAM TC-CQDs, TPTC or TPTC/LAAM TC-CQDs.

FIG. 46. NIR FL images of nude mice with bearing HeLa tumors right after the intravenous injection of TPTC or TPTC/LAAM TC-CQDs during 10 h.

FIG. 47. Ex vivo NIR FL imaging of major organs (heart, liver, spleen, lung and kidney) and tumors from nude mice bearing HeLa tumors after the intravenous injection of TPTC or TPTC/LAAM TC-CQDs at 8 h post-injection.

FIG. 48. Relative survival rate of mice after different treatments.

FIG. 49. Representative images of nude mice with hepatic (A549) tumor left and ovarial (HeLa) tumor right from different groups at different days after treatment.

FIG. 50. The A549 tumor (a) and HeLa tumor (b) growth curves of mice treated with saline, TPTC or TPTC/LAAM TC-CQDs.

FIG. 51. Body weights were measured during the 15 day evaluation period in mice treated with saline, TPTC or TPTC/LAAM TC-CQDs.

FIG. 52. Histological evaluation of major organs from mice treated with saline, TPTC, or TPTC/LAAM TC-CQDs. Each organ was sliced for H&E staining. The scale bar is 200 m.

FIG. 53. The complete blood panel data from healthy control and treated mice, including WBC (a), RBC (b), HGB (c), HCT (d), MCV (e), MCH (f), MCHC (g), PLT (h), RDW (i), PDW (j), MPV (k) and PCT (1). All the parameters of blood analysis fell well in the normal range. No significant difference in all blood test data was noticed between control and treated groups. Error bars were based on five mice per group.

FIG. 54. Serum biochemical parameters measurements. (a) Three indicators including ALT, AST and ALP for hepatic function, and another two, UREA (b) and CREA (c) for renal function were evaluated via blood samples from healthy control and treated mice.

FIG. 55. Preparation of NH₂ null LAAM TC-CQDs and COOH null LAAM TC-CQDs.

FIG. 56. FL emission spectra of NH₂ null LAAM TC-CQDs (a) and COOH null LAAM TC-CQDs (b). (c) The normalized FL emission spectra of LAAM TC-CQDs (kex=600 nm), NH₂ null LAAM TC-CQDs (λex=580 nm) and COOH null LAAM TC-CQDs (λex=600 nm), respectively.

FIG. 57. The FT-IR spectra of NH₂ null LAAM TC-CQDs (a) and COOH null LAAM TC-CQDs (b).

FIG. 58. The uptake of LAAM TC-CQDs, NH₂ null LAAM TC-CQDs and COOH null LAAM TC-CQDs in HeLa and CCC-ESF-1 cells obtained by flow cytometric profiles.

FIG. 59. Preparation of (a) 1,4-CQDs, (b) 1,5-CQDs and (c) 2,6-CQDs by hydrothermal treatment of CA and 1,4-DAAQ, 1,5-DAAQ or 2,6-DAAQ, respectively.

FIG. 60. TEM image of 1,4-CQDs (a), 1,5-CQDs (b) and 2,6-CQDs (c). AFM image of 1,4-CQDs (d), 1,5-CQDs (e) and 2,6-CQDs (f) on a Si substrate. The inset graphs in FIG. 65d-e are height profiles along the lines shown.

FIG. 61. Raman (a), XRD (b) and FT-IR spectra of 1,4-CQDs, 1,5-CQDs and 2,6-CQDs.

FIG. 62. XPS survey (a), C1s (b, c, d) and N1s (e, f, g) spectra of 1,4-CQDs, 1,5-CQDs and 2,6-CQDs.

FIG. 63. (a) UV-vis absorption and FL spectra of 1,4-CQDs (b), 1,5-CQDs (c) and 2,6-CQDs (d), respectively.

FIG. 64. The optimized electron delocalization molecular orbital (MO) diagrams of one FL unit of (a) 1,4-CQDs, (b) 1,5-CQDs and (c) 2,6-CQDs obtained from theoretical calculation with density functional theory calculations (B3LYP/6-31G(d,p)).

FIG. 65. The uptake of LAAM TC-CQDs, 1,4-CQDs, 1,5-CQDs and 2,6-CQDs in HeLa and CCC-ESF-1 cells obtained by flow cytometric profiles.

FIG. 66. (a) Preparation of Phe-CQDs via solvothermal method by using Phe and ethanol as precursors.

FIG. 67. The TEM (a) and HRTEM (b) image of Phe-CQDs.

FIG. 68. FT-IR (a), XPS survey (b), C1s (c), N1s (c), and O1s (e) spectra of Phe-CQDs.

FIG. 69. The UV-vis absorption (a) and FL emission (b) spectra of Phe-CQDs.

FIG. 70. The uptake of LAAM TC-CQDs and Phe-CQDs in HeLa and CCC-ESF-1 cells obtained by flow cytometric profiles.

FIG. 71a, Viability of HeLa and CCC-ESF-1 cells after treatment with LAAM TC-CQDs, TPTC or TPTC/LAAM TC-CQDs.

FIG. 71b, Changes in serum concentration of TPTC, when delivered in form of free drug or with TPTC/LAAM TC-CQDs, with time, in mice bearing HeLa tumors (n=5). Data are expressed as percentage of total injected dose (% ID).

FIG. 71c, Biodistribution of TPTC and TPTC/LAAM TC-CQDs in indicated organs isolated from mice bearing HeLa tumours (n=5).

FIG. 71d, Changes of tumour volume with time in HeLa tumour-bearing mice receiving the indicated treatments in mice bearing HeLa tumours (n=5).

FIG. 71e. Viability of A549 cells after treatment with LAAM TC-CQDs, TPTC or TPTC/LAAM TC-CQDs.

FIG. 71f, Changes in serum concentration of TPTC, when delivered in form of free drug or with TPTC/LAAM TC-CQDs, with time in mice bearing A549 tumours (n=5).

FIG. 71g, Biodistribution of TPTC and TPTC/LAAM TC-CQDs in indicated organs isolated from mice bearing A549 tumours (n=5).

FIG. 71h, Changes of tumour volume with time in A549 tumour-bearing mice receiving the indicated treatments (n=5).

FIG. 72. (a) FL emission spectrum of LAAM TC-CQDs at selected pHs. (b) FL emission spectrum of LAAM TC-CQDs in the presence of amino acids ((Asp, Glu, Met, Phe, Tyr and Gly)) or MMP-2.

FIG. 73. Photostability of LAAM TC-CQDs after long time storage or irradiation. (a) PL spectra of freshly prepared LAAM TC-CQDs solution (black) and LAAM TC-CQDs three months after storage at room temperature (red). (b) Change of ratio of PL intensity of LAAM TC-CQDs after irradiation at the indicated time point (F) to that of fresh LAAM TC-CQDs (FO) over time.

FIG. 74. Characterization of the impact of 5 hours UV irradiation at 365 nm on LAAM TC-CQDs. (a) Fluorescence emission spectra of LAAM TC-CQDs before (black) and after (red) UV irradiation (b) UV-vis spectra of LAAM TC-CQDs before (black) and after (red) UV irradiation with treatment of ninhydrin. (c) Flow cytometry analysis of uptake of control and UV irradiation-treated LAAM TC-CQDs in HeLa cells and CCC-ESF-1 cells.

FIG. 75. (a) Size distributions of fresh LAAM TC-CQDs, and (b) LAAM TC-CQDs after incubation in PBS buffer containing 10% FBS for 30 days. (c) Change of ratio of PL intensity of LAAM TC-CQDs after incubation in PBS buffer containing 10% FBS at the indicated time point (F) to that of fresh LAAM TC-CQDs (FO) over time.

### DETAILED DESCRIPTION

General Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, “a” or “an” means “at least one” or “one or more”.

The term “alkyl” as used herein refers to saturated hydrocarbon groups in a straight, branched, or cyclic configuration or any combination thereof, and particularly contemplated alkyl groups include those having ten or less carbon atoms, especially 1-6 carbon atoms and lower alkyl groups having 1-4 carbon atoms. Exemplary alkyl groups are methyl, ethyl, propyl, isopropyl, butyl, sec-butyl, tertiary butyl, pentyl, isopentyl, hexyl, cyclopropylmethyl, etc.

Alkyl groups can be unsubstituted, or they can be substituted to the extent that such substitution makes sense chemically. Typical substituents include, but are not limited to, halo, ═O, ═N—CN, ═N—OR^{a}, ═NR^{a}, —OR, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C₁-C₅ alkyl, C₂-C₅ heteroalkyl, 3-8 membered heterocyclyl, C₄-C₁₀ heterocyclyclalkyl, C₁-C₈ acyl, C₂-C₈ heteroacyl, C₂-C₈ alkenyl, C₂-C₈ heteroalkenyl, C₂-C₈ alkynyl, C₂-C₅ heteroalkynyl, C₆-C₁₀ aryl, or 5-10 membered heteroaryl, and each R^{a} is optionally substituted with halo, ═O, ═N—CN, ═N—OR^{b}, ═NR^{b}, OR^{b}, NR^{b}₂, SR^{b}, SO₂R^{b}, SO₂NR^{b}₂, NR^{b}SO₂R^{b}, NR^{b}CONR^{b}₂, NR^{b}COOR^{b}, NR^{b}COR^{b}, CN, COOR^{b}, CONR^{b}₂, OOCR^{b}, COR^{b}, and NO₂, wherein each R^{b} is independently H, C₁-C₈ alkyl, C₂-C₈ heteroalkyl, C₃-C₈ heterocyclyl, C₄-C₁₀ heterocyclyclalkyl, C₁-C₈ acyl, C₂-C₈ heteroacyl, C₆-C₁₀ aryl or 5-10 membered heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group. Where a substituent group contains two R^{a} or R^{b} groups on the same or adjacent atoms (e.g., —NR^{b}2, or —NR^{b}—C(O) R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

The term “cycloalkyl” as used herein refers to a cyclic alkane (i.e., in which a chain of carbon atoms of a hydrocarbon forms a ring), preferably including three to eight carbon atoms. Thus, exemplary cycloalkanes include cyclopropyl, cyclobutyl, cyclopentyl, cyclohexyl, cycloheptyl, and cyclooctyl. Cycloalkyls also include one or two double bonds, which form the “cycloalkenyl” groups. Cycloalkyl groups are optionally substituted by groups suitable for alkyl groups as set forth herein.

The term “aryl” or “aromatic moiety” as used herein refers to an aromatic ring system, which may further include one or more non-carbon atoms. These are typically 5-6 membered isolated rings, or 8-10 membered bicyclic groups, and can be substituted. Thus, contemplated aryl groups include (e.g., phenyl, naphthyl, etc.) and pyridyl, and pyridone rings are considered aromatic for discussion herein. Further contemplated aryl groups may be fused (i.e., covalently bound with 2 atoms on the first aromatic ring) with one or two 5- or 6-membered aryl or heterocyclic group, and are thus termed “fused aryl” or “fused aromatic”.

The term “partially aromatic” as used herein refers to a ring that is fused to and shares at least one bond with an aromatic ring. Partially aromatic fused ring systems are those in which each ring is either aromatic or partially aromatic.

The term “partially unsaturated” as used herein refers to a ring containing at least one double bond within the cyclic ring, where the ring is not aromatic. Note that a ring that is partially aromatic shares an aromatic bond, and because of the aromatic ring, it is considered to be partially unsaturated as well as partially aromatic.

Aromatic groups containing one or more heteroatoms (typically N, O or S) as ring members can be referred to as heteroaryl or heteroaromatic groups. Typical heteroaromatic groups include monocyclic C₅-C₆ aromatic groups such as pyridyl, pyrimidyl, pyrazinyl, thienyl, furanyl, pyrrolyl, pyrazolyl, thiazolyl, oxazolyl, isothiazolyl, isoxazolyl, and imidazolyl and the fused bicyclic moieties formed by fusing one of these monocyclic groups with a phenyl ring or with any of the heteroaromatic monocyclic groups to form a 8-10 membered bicyclic group such as indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms.

As also used herein, the terms “heterocycle” and “heterocyclic” as used herein refer to any compound in which a plurality of atoms form a ring via a plurality of covalent bonds, wherein the ring includes at least one atom other than a carbon atom as a ring member.

Particularly contemplated heterocyclic rings include 5- and 6-membered rings with nitrogen, sulfur, or oxygen as the non-carbon atom (e.g., imidazole, pyrrole, triazole, dihydropyrimidine, indole, pyridine, thiazole, tetrazole etc.). Typically these rings contain 0-1 oxygen or sulfur atoms, at least one and typically 2-3 carbon atoms, and up to four nitrogen atoms as ring members. Further contemplated heterocycles may be fused (i.e., covalently bound with two atoms on the first heterocyclic ring) to one or two carbocyclic rings or heterocycles, and are thus termed “fused heterocycle” or “fused heterocyclic ring” or “fused heterocyclic moieties” as used herein. Where the ring is aromatic, these can be referred to herein as ‘heteroaryl’ or heteroaromatic groups.

Heterocyclic groups that are not aromatic can be substituted with groups suitable for alkyl group substituents, as set forth above.

Aryl and heteroaryl groups can be substituted where permitted. Suitable substituents include, but are not limited to, halo, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C₁-C₈ alkyl, C₂-C₈ heteroalkyl, C₃-C₈ heterocyclyl, C4-C10 heterocyclyclalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or 5-10 membered heteroaryl, and each R^{a} is optionally substituted with halo, ═O, ═N—CN, ═N—OR^{b}, ═NR^{b}, OR^{b}, NR^{b}₂, SR^{b}, SO₂R^{b}, SO₂NR^{b}₂. NR^{b}SO₂R^{b}, NR^{b}CONR^{b}₂, NR^{b}COOR^{b}, NR^{b}COR^{b}, CN, COOR^{b}, CONR^{b}₂, OOCR^{b}, COR^{b}, and NO₂, wherein each R^{b} is independently H, C₁-C₈ alkyl, C₂-C₈ heteroalkyl, C₃-C₈ heterocyclyl, C₄-C₁₀ heterocyclyclalkyl, C₁-C₈ acyl, C₂-C₈ heteroacyl, C₆-C₁₀ aryl or 5-10 membered heteroaryl. Alkyl, alkenyl and alkynyl groups can also be substituted by C₁-C₈ acyl, C₂-C₈ heteroacyl, C₆-C₁₀ aryl or 5-10 membered heteroaryl, each of which can be substituted by the substituents that are appropriate for the particular group. Where a substituent group contains two R^{a} or R^{b} groups on the same or adjacent atoms (e.g., —NR^{b}₂, or —NR^{b}—C(O) R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

The term “alkoxy” as used herein refers to a hydrocarbon group connected through an oxygen atom, e.g., —O-Hc, wherein the hydrocarbon portion He may have any number of carbon atoms, typically 1-10 carbon atoms, may further include a double or triple bond and may include one or two oxygen, sulfur or nitrogen atoms in the alkyl chains, and can be substituted with aryl, heteroaryl, cycloalkyl, and/or heterocyclyl groups. For example, suitable alkoxy groups include methoxy, ethoxy, propyloxy, isopropoxy, methoxyethoxy, benzyloxy, allyloxy, and the like. Similarly, the term “alkylthio” refers to alkylsulfides of the general formula —S-Hc, wherein the hydrocarbon portion He is as described for alkoxy groups. For example, contemplated alkylthio groups include methylthio, ethylthio, isopropylthio, methoxyethylthio, benzylthio, allylthio, and the like.

The term ‘amino’ as used herein refers to the group —NH₂. The term “alkylamino” refers to amino groups where one or both hydrogen atoms are replaced by a hydrocarbon group He as described above, wherein the amino nitrogen “N” can be substituted by one or two He groups as set forth for alkoxy groups described above. Exemplary alkylamino groups include methylamino, dimethylamino, ethylamino, diethylamino, etc. Also, the term “substituted amino” refers to amino groups where one or both hydrogen atoms are replaced by a hydrocarbon group He as described above, wherein the amino nitrogen “N” can be substituted by one or two He groups as set forth for alkoxy groups described above.

The term “halogen” as used herein refers to fluorine, chlorine, bromine and iodine. Where present as a substituent group, halogen or halo typically refers to F or Cl or Br, more typically F or Cl.

The term “haloalkyl” refers to an alkyl group as described above, wherein one or more hydrogen atoms on the alkyl group have been substituted with a halo group. Examples of such groups include, without limitation, fluoroalkyl groups, such as fluoroethyl, trifluoromethyl, difluoromethyl, trifluoroethyl and the like.

The term “haloalkoxy” refers to the group alkyl-O— wherein one or more hydrogen atoms on the alkyl group have been substituted with a halo group and include, by way of examples, groups such as trifluoromethoxy, and the like.

It should further be recognized that all of the above-defined groups may further be substituted with one or more substituents, which may in turn be substituted with hydroxy, amino, cyano, C₁-C₄ alkyl, halo, or C₁-C₄ haloalkyl. For example, a hydrogen atom in an alkyl or aryl can be replaced by an amino, halo or C₁-C₄ haloalkyl or alkyl group.

The term “substituted” as used herein refers to a replacement of a hydrogen atom of the unsubstituted group with a functional group, and particularly contemplated functional groups include nucleophilic groups (e.g., —NH₂, —OH, —SH, —CN, etc.), electrophilic groups (e.g., C(O)OR, C(X)OH, etc.), polar groups (e.g., —OH), non-polar groups (e.g., heterocycle, aryl, alkyl, alkenyl, alkynyl, etc.), ionic groups (e.g., —NH₃⁺), and halogens (e.g., —F, —Cl), NHCOR, NHCONH₂, OCH₂COOH, OCH₂CONH₂, OCH₂CONHR, NHCH₂COOH, NHCH₂CONH₂, NHSO₂R, OCH₂-heterocycles, PO₃H, SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, compounds arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The compounds and CQDs of the invention possess at least one alpha-amino carboxylic acid moiety, and thus are capable of forming salts by protonation (acid addition) or by deprotonation; moreover, they can exist as zwitterions, as is known in the art for amino acid compounds. It is to be understood that, for convenience, unless otherwise specified, each reference to the compounds of the invention and the CQDs of the invention, the substance can be made, isolated, and used in any suitable protonation state, as a pharmaceutically acceptable salt or as a zwitterion. In use or in solution, the protonation state largely depends upon the pH of its environment, and interconversion of the protonation states is well understood. Also, where a compound or CQD of the invention has more than one alpha-amino carboxylic acid moiety, each of the alpha-amino acid moieties can be in any of these forms, depending on the environment, so the compound can comprise an internal mixture of salts and/or zwitterionic moieties.

The term “salt thereof” means a compound formed when a proton of an acid is replaced by a cation, such as a metal cation or an organic cation and the like; or a basic group such as a nitrogen atom (especially nitrogen that is sp³ hybridized) accepts a proton to form a positively charged group (e.g., ammonium), in which case the positively charged compound is accompanied by an anionic counterion such as a halide anion. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

The terms “polypeptide,” “oligopeptide,” “peptide,” and “protein” are used interchangeably herein to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the terms “variant” is used in reference to polypeptides that have some degree of amino acid sequence identity to a parent polypeptide sequence. A variant is similar to a parent sequence, but has at least one substitution, deletion or insertion in their amino acid sequence that makes them different in sequence from a parent polypeptide. Additionally, a variant may retain the functional characteristics of the parent polypeptide, e.g., maintaining a biological activity that is at least 50%, 60%, 70%, 80%, 90%, 95%, 98%, or 99% of that of the parent polypeptide.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule, and can be an immunoglobulin of any class, e.g., IgG, IgM, IgA, IgD and IgE. IgY, which is the major antibody type in avian species such as chicken, is also included within the definition. As used herein, the term encompasses not only intact polyclonal or monoclonal antibodies, but also fragments thereof (such as Fab, Fab′, F(ab′)2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein, the term “antigen” refers to a target molecule that I s specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein, the term “epitope” refers to a portion of an antigen, e.g., a peptide sequence of at least about 3 to 5, preferably about 5 to 10 or 15, and not more than about 1,000 amino acids (or any integer there between), which define a sequence that by itself or as part of a larger sequence, binds to an antibody generated in response to such sequence. There is no critical upper limit to the length of the fragment, which may, for example, comprise nearly the full-length of the antigen sequence, or even a fusion protein comprising two or more epitopes from the target antigen. An epitope for use in the subject invention is not limited to a peptide having the exact sequence of the portion of the parent protein from which it is derived, but also encompasses sequences identical to the native sequence, as well as modifications to the native sequence, such as deletions, additions and substitutions (conservative in nature).

As used herein, the term “specifically binds” refers to the binding specificity of a specific binding pair. Recognition by an antibody of a particular target in the presence of other potential targets is one characteristic of such binding. Specific binding involves two different molecules wherein one of the molecules specifically binds with the second molecule through chemical or physical means. The two molecules are related in the sense that their binding with each other is such that they are capable of distinguishing their binding partner from other constituents having similar characteristics. The members of the binding component pair are referred to as ligand and receptor (anti-ligand), specific binding pair (SBP) member and SBP partner, and the like. A molecule may also be an SBP member for an aggregation of molecules; for example an antibody raised against an immune complex of a second antibody and its corresponding antigen may be considered to be an SBP member for the immune complex.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

As used herein, the term “homologue” is used to refer to a nucleic acid which differs from a naturally occurring nucleic acid (e.g., the “prototype” or “wild-type” nucleic acid) by minor modifications to the naturally occurring nucleic acid, but which maintains the basic nucleotide structure of the naturally occurring form. Such changes include, but are not limited to: changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art for the production of nucleic acids including, but not limited to, recombinant DNA techniques, chemical synthesis, etc.

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5.times.Denhardt's solution, 5.times.SSPE, 0.2% SDS at 42.degree. C., followed by washing in 0.2.times.SSPE, 0.2% SDS, at 42.degree. C. High stringency conditions can be provided, for example, by hybridization in 50% formamide, 5.times.Denhardt's solution, 5.times.SSPE, 0.2% SDS at 42.degree. C., followed by washing in 0.1.times.SSPE, and 0.1% SDS at 65.degree. C. Low stringency hybridization refers to conditions equivalent to hybridization in 10% formamide, 5.times.Denhardt's solution, 6.times.SSPE, 0.2% SDS at 22.degree. C., followed by washing in 1.times.SSPE, 0.2% SDS, at 37.degree. C. Denhardt's solution contains 1% Ficoll, 1% polyvinylpyrolidone, and 1% bovine serum albumin (BSA). 20.times.SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art.

As used herein, the term “RNA interference” or “RNAi” refers generally to a process in which a double-stranded RNA molecule or a short hairpin RNA molecule reducing or inhibiting the expression of a nucleic acid sequence with which the double-stranded or short hairpin RNA molecule shares substantial or total homology. The term “short interfering RNA” or “siRNA” or “RNAi agent” refers to an RNA (or RNA analog) sequence comprising between about 10-50 nucleotides (or nucleotide analogs) that elicits RNA interference. See Kreutzer et al., WO 00/44895; Zernicka-Goetz et al., WO 01/36646; Fire, WO 99/32619; Mello & Fire, WO 01/29058. As used herein, siRNA molecules include RNA molecules encompassing chemically modified nucleotides and non-nucleotides. The term “ddRNAi agent” refers to a DNA-directed RNAi agent that is transcribed from an exogenous vector. The terms “short hairpin RNA” or “shRNA” refer to an RNA structure having a duplex region and a loop region. In certain embodiments, ddRNAi agents are expressed initially as shRNAs.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent or treatment, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art. In some embodiments, “treatment” means any manner in which the symptoms of a condition, disorder or disease are ameliorated or otherwise beneficially altered. Treatment also encompasses any pharmaceutical use of the compositions herein. In some embodiments, “amelioration” of the symptoms of a particular disorder by administration of a particular pharmaceutical composition refers to any lessening, whether permanent or temporary, lasting or transient that can be attributed to or associated with administration of the composition.

The term “prediction” or “prognosis” is used herein to refer to the likelihood that a patient will respond either favorably or unfavorably to a drug or set of drugs, or the likely outcome of a disease. In one embodiment, the prediction relates to the extent of those responses or outcomes. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc.

As used herein, “production by recombinant means” refers to production methods that use recombinant nucleic acid methods that rely on well-known methods of molecular biology for expressing proteins encoded by cloned nucleic acids.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20.sup.th ed., (Lippincott, Williams & Wilkins 2003). Except insofar as any conventional media or agent is incompatible with the active compound or substance, such use in the compositions is contemplated.

As used herein, the term “therapeutically effective amount” or “effective amount” refers to an amount of a therapeutic agent that when administered alone or in combination with an additional therapeutic agent to a cell, tissue, or subject is effective to prevent or ameliorate a disease or disorder in a subject. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. In some embodiment, “an effective amount of a compound or substance for treating a particular disease” is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

As used herein, a “prodrug” is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

The compounds and compositions described herein can be administered to a subject in need of treatment for a cell proliferation disorder such as cancer, particularly cancers selected from leukemia, lymphoma, lung cancer, colon cancer, melanoma, ovarian cancer, renal cancer, prostate cancer, breast cancer, head and neck cancers, and pancreatic cancer. The subject is typically a mammal diagnosed as being in need of treatment for one or more of such proliferative disorders, and frequently the subject is a human. The methods comprise administering an effective amount of at least one compound of the invention; optionally the compound may be administered in combination with one or more additional therapeutic agents, particularly therapeutic agents known to be useful for treating the cancer or proliferative disorder afflicting the particular subject.

The carbon quantum dots of the invention are particles having suitable size and electronic characteristics to function as quantum dots, and are comprised of at least 20% by weight carbon, and preferably at least 50% by weight carbon. Where a CQD is described as ‘comprising’ a compound of a specified structure, it is understood that these carbon quantum dots do not necessarily consist of a single pure compound. The CQD particle may contain other materials within the particulate core, and the core may produce or influence the quantum dot properties (fluorescence, photoacoustic) of the particle; but the particle nonetheless contains the specified compound, and its valuable biological properties (binding to LAT1, internalization into cells, selectivity for tumors or specific LAT1-expressing cells over other cells) are attributable to the specified chemical structure as demonstrated by data herein showing that modification of the specified chemical structures of the CQDs of the embodiments and claims herein results in decrease in these valuable biological properties. Thus, for example, the CQDs as described and claimed herein with reference to a specific chemical structure are particles that contain at least a biologically relevant amount of the specific chemical structure, and may contain 5% by weight, or 10% by weight of the specific chemical structure. The CQDs may also, of course, consist of or consist essentially of the specified compound(s) in a particular embodiment or claim. Likewise, methods described herein may produce compositions that contain at least 5% or at least 10% by weight of the compound described as being comprised in the product. These products may be obtained as particles, or may be capable of forming particles having the properties of a carbon quantum dots, but it is also to be understood that the compounds and compositions of the invention, including products made by the processes described and claimed, and particles comprising these compounds and compositions are expected to exhibit useful affinity for LAT1 and ability to be selectively internalized through LAT1 into cells that express LAT1, even if not sized to function as CQDs. These compounds and compositions are thus useful to internalize labels or therapeutic agents into LAT1-expressing cells and thus exhibit valuable pharmaceutical, imaging, and therapeutic utility; thus, where the materials herein are not expressly described as carbon quantum dots, the invention includes compounds, compositions and products described herein, whether or not they are in the form of particles of function as carbon quantum dots.

The following enumerated embodiments represent some aspects of the invention.

1. A carbon quantum dot, which:
a) comprises a core structure comprising a fused polycyclic array of 6-membered rings, each of which is aromatic or unsaturated, wherein the polycyclic array of 6-membered rings is substituted with at least one C₁-C₃ alkyl group that is substituted with at least a carboxyl group and an amino group; or
b) comprises a fused polycyclic ring system comprising 6-membered rings, wherein each ring is aromatic or unsaturated, and the polycyclic ring system is substituted with at least one group of Formula A:

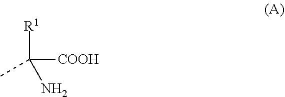

wherein R¹ is H or a C₁-C₃ alkyl group optionally substituted with one or two groups selected from halo, —OH, —OMe, —NH₂, —SMe, —COOH, and —CONH₂, and the dashed bond indicates where said group of Formula A is connected to said fused polycyclic ring system; or
c) comprises a polycyclic aromatic or partially aromatic ring system comprising at least 10 fused 6-membered rings, wherein said polycyclic aromatic or partially aromatic ring system is fused to at least one subunit of Formula B:

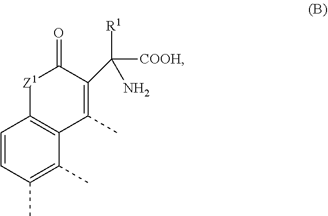

wherein:
R¹ is H or a C₁-C₃ alkyl optionally substituted with one or two groups selected from halo, —OH, —OMe, —NH₂, —SMe, —COOH, and —CONH₂,
Z¹ is NR² or C(R²)₂, where each R² is independently selected from H and C₁-C₃ alkyl, and wherein the dashed bonds indicate where said Formula B is fused to said polycyclic aromatic or partially aromatic ring system; and/or
d) is configured to selectively enter a cell that expresses a large neutral amino acid transporter (LAT1), or a subunit thereof; and/or
e) is configured to selectively enter a tumor or cancer cell,
   wherein, preferably, the percentage of carbon atoms by weight in said carbon quantum dot is 20% or more. In some of these embodiments, the percentage of carbon atoms by weight in said carbon quantum dot can be 20%, 25%, 30%, 35%, 40%, 45%, 50%, 55%, 60%, 65%, 70%, 75%. 80%, 85%, 90%, 91%, 92%, 93%, 94%, 95%, 96%, 97%, 98%, 99% or more, or any subrange thereof.

In some examples of this embodiment, the carbon quantum dot comprises a core structure comprising a fused polycyclic array of 6-membered rings, each of which is aromatic or unsaturated (partially aromatic), wherein the polycyclic array of 6-membered rings is substituted with at least one C₁-C₃ alkyl group that is substituted with at least a carboxyl group and an amino group; and the polycyclic array of 6-membered rings can optionally be substituted with one or more additional groups selected from C₁₋₄ alkyl, phenyl, C₁₋₄ alkoxy, halo, COOR*, —OH, CN, and —NR*₂, where each R* is independently selected from H and C₁₋₃ alkyl, where each C₁₋₃ alkyl is optionally substituted with one to three groups selected from —OH, —COOH, —NH₂, and C₁₋₂ alkyl. Moreover, the C₁-C₃ alkyl group that is substituted with at least a carboxyl group and an amino group can optionally be further substituted by a C₁-C₂ group that is optionally substituted with one to three groups selected from —OH, —COOH, —NH₂, and C₁₋₂ alkyl.

In some examples of the first embodiment, the carbon quantum dot comprises a fused polycyclic ring system comprising 6-membered rings, wherein each ring is aromatic or unsaturated, and the polycyclic ring system is substituted with at least one group of Formula A:

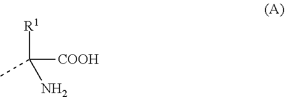

wherein R¹ is H or a C₁-C₃ alkyl group optionally substituted with one or two groups selected from halo, —OH, —OMe, —NH₂, —SMe, —COOH, and —CONH₂, and the dashed bond indicates where said group of Formula A is connected to said fused polycyclic ring system. In these compounds and CQDs, the polycyclic ring system is optionally further substituted with one or more groups selected from C₁₋₄ alkyl, phenyl, C₁₋₄ alkoxy, halo, COOR*, —OH, CN, and —NR*₂, where each R* is independently selected from H and C₁₋₃ alkyl, where each C₁₋₃ alkyl is optionally substituted with one to three groups selected from —OH, —COOH, —NH₂, and C₁₋₂ alkyl. Moreover, the C₁-C₃ alkyl group that is substituted with at least a carboxyl group and an amino group can optionally be further substituted by a C₁-C₂ group that is optionally substituted with one to three groups selected from —OH, —COOH, —NH₂, and C₁₋₂ alkyl. In preferred examples of these compounds and CQDs, R¹ is H, methyl, or —CH₂COOH.

In some examples of embodiment 1, the carbon quantum dot comprises a polycyclic aromatic or partially aromatic ring system comprising at least 10 fused 6-membered rings, wherein said polycyclic aromatic or partially aromatic ring system is fused to at least one subunit of Formula B:

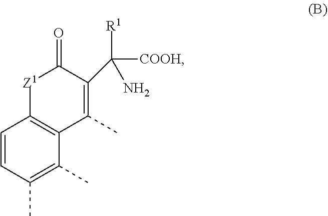

wherein:
R¹ is H or a C₁-C₃ alkyl optionally substituted with one or two groups selected from halo, —OH, —OMe, —NH₂, —SMe, —COOH, and —CONH₂,
Z¹ is NR² or C(R²)₂, where each R² is independently selected from H and C₁-C₃ alkyl, and wherein the dashed bonds indicate where said Formula B is fused to said polycyclic aromatic or partially aromatic ring system. In these compounds and CQDs, the polycyclic ring system is optionally further substituted with one or more groups selected from C₁₋₄ alkyl, phenyl, C₁₋₄ alkoxy, halo, COOR*, —OH, CN, and —NR*₂, where each R* is independently selected from H and C₁₋₃ alkyl, where each C₁₋₃ alkyl is optionally substituted with one to three groups selected from —OH, —COOH, —NH₂, and C₁₋₂ alkyl. Moreover, the C₁-C₃ alkyl group that is substituted with at least a carboxyl group and an amino group can optionally be further substituted by a C₁-C₂ group that is optionally substituted with one to three groups selected from —OH, —COOH, —NH₂, and C₁₋₂ alkyl.

In some examples of embodiment 1, including the preceding examples of these embodiments, the carbon quantum dot is configured to selectively enter a cell that expresses a large neutral amino acid transporter (LAT1), or a subunit thereof. In these examples, the CQDs are preferably adapted or configured to exhibit selective uptake into a cell, having an uptake rate of at least 90%, e.g., at least 90%, 91%, 92%, 93%, 94%, 95%, 96%, 97%, 98%, 99% or more.

In some examples of embodiment 1, including the preceding examples of these embodiments, the carbon quantum dot is configured to selectively enter a tumor or cancer cell. In these examples, the tumor or cancer cell expresses a transporter that recognizes and binds to the carbon quantum dot.

2. The carbon quantum dot of embodiment 1, which comprises a core structure comprising a fused polycyclic array of 6-membered rings, each of which is aromatic or unsaturated, wherein the polycyclic array of 6-membered rings is substituted with at least one C₁-C₃ alkyl group that is substituted with a carboxyl group and an amino group.
3. The carbon quantum dot of embodiment 2, wherein the core structure comprises at least 5 fused 6-membered aromatic or unsaturated core rings.
4. The carbon quantum dot of embodiment 2 or 3, wherein the polycyclic array of 6-membered rings is substituted with at least two of the C₁-C₃ alkyl groups each of which is substituted with a carboxyl group and an amino group, and is optionally further substituted by one or two groups selected from —OH, methyl, phenyl, —OMe, —NH₂, —SMe, —COOH, and —CONH₂.
5. The carbon quantum dot of any of embodiments 2-4, wherein a ratio between the number of the 6-membered aromatic or unsaturated rings in the core structure and the number of the C₁-C₃ alkyl groups is at least 2.
6. The carbon quantum dot of embodiment 5, wherein the ratio between the number of the 6-member aromatic or unsaturated rings in the core structure and the number of the C₁-C₃ alkyl groups is from about 2 to about 70, e.g., 2, 3, 4, 5, 6, 7, 8, 9, 10, 15, 20, 25, 30, 35, 40, 45, 50, 55, 60, 65 or 70, or any subrange thereof.
7. The carbon quantum dot of embodiment 1, which comprises a fused polycyclic ring system comprising 6-membered rings, wherein each ring is aromatic or unsaturated, and the polycyclic ring system is substituted with at least one group of Formula A:

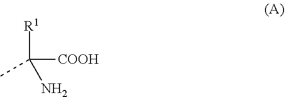

wherein R¹ is H or a C₁-C₃ alkyl group optionally substituted with one or two groups selected from —OH, —OMe, —SMe, —COOH, and —CONH₂, and the dashed bond indicates where said group of Formula (A) is connected to said fused polycyclic ring system.
8. The carbon quantum dot of embodiment 7, wherein R¹ is H.
9. The carbon quantum dot of embodiment 7, wherein R¹ is a C₁-C₃ alkyl group.
10. The carbon quantum dot of embodiment 9, wherein R¹ is a C₁-C₃ alkyl group substituted with one or two groups selected from halo, —OH, —OMe, —SMe, —COOH, and —CONH₂.
11. The carbon quantum dot of embodiment 1, which comprises a polycyclic aromatic or partially aromatic ring system comprising at least 10 fused 6-membered rings, wherein the polycyclic aromatic or partially aromatic ring system is fused to at least one subunit of Formula B:

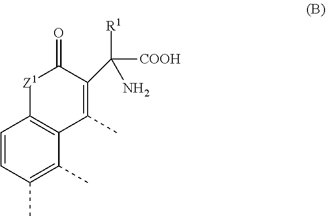

wherein:
R¹ is H or a C₁-C₃ alkyl optionally substituted with one or two groups selected from halo, —OH, —OMe, —SMe, —COOH, and —CONH₂,
Z¹ is NR² or C(R²)₂, where each R² is independently selected from H and C₁-C₃ alkyl, and
   wherein the dashed bonds indicate where the Formula B is fused to the polycyclic aromatic ring system.
   12. The carbon quantum dot of embodiment 11, wherein R¹ is H.
   13. The carbon quantum dot of embodiment 11, wherein R¹ is a C₁-C₃ alkyl.
   14. The carbon quantum dot of embodiment 11, wherein R¹ is a C₁-C₃ alkyl substituted with one or two groups selected from halo, —OH, —OMe, —SMe, —COOH, and —CONH₂. 15. The carbon quantum dot of any of embodiments 11-14, wherein Z¹ is NR², e.g., NH.
   16. The carbon quantum dot of any of embodiments 11-14, wherein Z¹ is C(R²)₂, e.g., CH₂. 17. The carbon quantum dot of embodiment 15 or 16, wherein R² is H.
   18. The carbon quantum dot of embodiment 15 or 16, wherein R² is a C₁-C₃ alkyl.
   19. The carbon quantum dot of any of embodiments 11-18, which comprises at least two subunits of Formula B, e.g., four subunits of Formula B, fused to the polycyclic aromatic or partially aromatic ring system.
   20. The carbon quantum dot of any of embodiments 11-19, which has a molecular weight from about 500 to about 500,000, e.g., about 500, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, or any subrange thereof.
   21. The carbon quantum dot of any of embodiments 1-20, which is formed by reacting at least two different precursors, at least one precursor comprising carboxyl and hydroxyl groups, and at least another precursor comprising a plurality of 6-member aromatic rings and at least two amino groups.
   22. The carbon quantum dot of embodiment 21, wherein the at least one precursor comprising carboxyl and hydroxyl groups is citric acid (CA).
   23. The carbon quantum dot of embodiment 21 or 22, wherein the precursor comprising a plurality of 6-member aromatic rings is 1,4,5,8-tetraminoanthraquinone (TAAQ), 1,4-diaminoanthraquinone (1,4-DAAQ), or 1,5-diaminoanthraquinone (1,5-DAAQ).
   24. The carbon quantum dot of any of embodiments 1-20, which is formed by reacting at least two different precursors, at least one precursor comprising an alpha-amino carboxylic acid compound or an alpha-hydroxy carboxylic acid compound, and a second precursor comprising a C₁-C₈ alcohol.
   25. The carbon quantum dot of embodiment 24, wherein the precursor comprising an alpha-amino carboxylic acid compound is phenylalanine (Phe).
   26. The carbon quantum dot of embodiment 24 or 25, wherein the second precursor is ethanol.
   27. The carbon quantum dot of any of embodiments 1-26, which is configured to selectively enter a cell that expresses a large neutral amino acid transporter (LAT1), or a subunit thereof.
   28. The carbon quantum dot of embodiment 27, wherein the LAT1 comprises 4F2hc/CD98 heavy subunit protein encoded by the SLC3A2 (solute carrier family 3 member 2) gene and CD98 light subunit protein encoded by the SLC7A5 gene.
   29. The carbon quantum dot of embodiment 27 or 28, wherein the cell has a relative uptake rate of a large neutral amino acid of at least 90%, or the cell has a LAT1 expression level at least 10× higher than normal human bone marrow stromal cells. For example, the cell can have a relative uptake rate of a large neutral amino acid of at least 90%, 91%, 92%, 93%, 94%, 95%, 96%, 97% 98%, 99% or more, or any subrange thereof. In another example, the cell can have a LAT1 expression level at least 10×, 15×, 20×, 25×, 30×, 35×, 40×, 45×, or 50×, higher than normal human bone marrow stromal cells, or any subrange thereof.
   30. The carbon quantum dot of any of embodiments 27-29, wherein the cell has a ratio between LAT1 gene expression level and expression level of another gene, e.g., a house keeping gene such as GAPDH, of at least 0.5. For example, the cell can have a ratio between LAT1 gene expression level and expression level of another gene, e.g., a house keeping gene such as GAPDH, of at least 0.5, 0.6, 0.7, 0.8, 0.9, 1.0 or higher, or any subrange thereof.
   31. The carbon quantum dot of any of embodiments 27-30, wherein the LAT1 preferentially internalizes a branched-chain amino acid and/or an aromatic amino acid.
   32. The carbon quantum dot of embodiment 31, wherein the branched-chain amino acid is a proteinogenic branched-chain amino acid, e._, leucine, isoleucine, or valine.
   33. The carbon quantum dot of embodiment 31, wherein the branched-chain amino acid is a non-proteinogenic branched-chain amino acid, e.g., 2-aminoisobutyric acid.
   34. The carbon quantum dot of embodiment 31, wherein the aromatic amino acid is a proteinogenic amino acid, e.g., phenylalanine, tyrosine, arginine, tryptophan or histidine.
   35. The carbon quantum dot of embodiment 31, wherein the aromatic amino acid is a non-proteinogenic aromatic amino acid, e.g., thyroxine, 5-hydroxytryptophan (5-HTP) or L-DOPA.
   36. The carbon quantum dot of any of embodiments 27-35, wherein the LAT1 is preferentially or highly expressed in a targeted organ relative to other tissue(s) of a subject, e.g., a mammal.
   37. The carbon quantum dot of any of embodiments 27-35, wherein the LAT1 is preferentially or highly expressed in tumor or cancer cells relative to other tissue(s) or cells of a subject, e.g., a mammal.
   38. The carbon quantum dot of any of embodiments 1-37, which is doped with N, S, P, B and/or O.
   39. The carbon quantum dot of any of embodiments 1-38, which has an average particle size or diameter ranging from about 0.2 nm to about 10 nm, e.g., from about 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, or 9 nm, to about 10 nm, or any subrange thereof. In many of these embodiments, the carbon quantum dot has an average particle between 1 nm and 5 nm, or between 2 nm and 4 nm.
   40. The carbon quantum dot of any of embodiments 1-39, which has an excitation wavelength ranging from about 300 nm to about 900 nm, e.g., about 300 nm, 400 nm, 500 nm, 600 nm, 700 nm, 800 nm, 900 nm, or any subrange thereof.
   41. The carbon quantum dot of any of embodiments 1-40, which has an emission wavelength ranging from about 400 nm to about 1,000 nm, e.g., about 400 nm, 500 nm, 600 nm, 700 nm, 800 nm, 900 nm, 1,000 nm, or any subrange thereof.
   42. The carbon quantum dot of embodiment 41, which emits near infrared (NIR) fluorescence (FL).
   43. The carbon quantum dot of any of embodiments 1-42, which is configured for photoacoustic (PA) imaging upon radiation.
   44. The carbon quantum dot of embodiment 43, wherein upon radiation at an excitation wavelength ranging from about 600 nm to about 900 nm, e.g., about 600 nm, 650 nm, 700 nm, 750 nm, 800 nm, 850 nm, or 900 nm, or any subrange thereof, the carbon quantum dot generates ultrasonic wave.
   45. The carbon quantum dot of embodiment 44, wherein the ultrasonic wave is configured for forming a PA image.
   46. The carbon quantum dot of any of embodiments 43-45, which has an increasing linearity at a concentration from about 0.1 μg/mL to about 10 μg/mL, e.g., about 0.1 μg/mL, 0.5 μg/mL, 1 g/mL, 2 μg/mL, 3 μg/mL, 4 μg/mL, 5 μg/mL, 6 μg/mL, 7 μg/mL, 8 μg/mL, 9 μg/mL, 10 μg/mL, or any subrange thereof.
   47. The carbon quantum dot of any of embodiments 1-46, which emits near infrared (NIR) fluorescence (FL) and is configured for photoacoustic (PA) imaging upon radiation.
   48. The carbon quantum dot of embodiment 47, which is configured for deep tissue, tumor or cancer imaging.
   49. The carbon quantum dot of any of embodiments 1-48, which is configured to selectively enter a tumor or cancer cell.
   50. The carbon quantum dot of embodiment 49, which is configured to selectively enter nucleus of a tumor or cancer cell.
   51. The carbon quantum dot of embodiment 49 or 50, wherein the tumor or cancer is selected from the group consisting of sarcoma, epidermoid cancer, fibrosarcoma, cervical cancer, gastric carcinoma, skin cancer, leukemia, lymphoma, lung cancer, non-small cell lung cancer, colon cancer, melanoma, ovarian cancer, renal cancer, prostate cancer, breast cancer, liver cancer, head and neck cancers, pancreatic cancer, bladder cancer, esophagus cancer, tongue cancer, and cutaneous malignant melanoma.
   52. The carbon quantum dot of any of embodiments 1-51, which is configured to selectively enter a cell of a tumor or cancer selected from the group consisting of sarcoma, epidermoid cancer, fibrosarcoma, cervical cancer, gastric carcinoma, skin cancer, leukemia, lymphoma, lung cancer, non-small cell lung cancer, colon cancer, melanoma, ovarian cancer, renal cancer, prostate cancer, breast cancer, liver cancer, head and neck cancers, pancreatic cancer, bladder cancer, esophagus cancer, tongue cancer, and cutaneous malignant melanoma.
   53. The carbon quantum dot of any of embodiments 1-52, which is configured to selectively enter a cell or nucleus of the cell in a subject.
   54. The carbon quantum dot of embodiment 53, wherein the subject is a mammal, e.g., a human or a non-human mammal.
   55. A method for preparing a polycyclic compound or particle comprising a polycyclic compound, e.g., a carbon quantum dot of any of embodiments 1-54, which method comprises solvothermal synthesis using at least two different precursors, wherein:
1) at least one precursor comprises an alpha-amino carboxylic acid compound or an alpha-hydroxy carboxylic acid compound, and at least one other precursor that comprises a plurality of 6-membered aromatic rings; or
2) at least one precursor comprising phenylalanine or a phenylalanine analog, e.g. an analog of phenylalanine having a substituent on the phenyl ring that is selected from halo, hydroxy, methoxy, methyl, and CF₃, and a C₁-C₈ alcohol.

In some of these embodiments, the polycyclic compound comprises a fused polycyclic ring system comprising 6-membered rings, wherein each ring is aromatic or unsaturated (partially aromatic), and the polycyclic ring system is substituted with at least one group of Formula A:

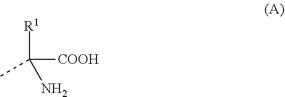

wherein R¹ is H or a C₁-C₃ alkyl group optionally substituted with one or two groups selected from halo, —OH, —OMe, —NH₂, —SMe, —COOH, and —CONH₂, and the dashed bond indicates where said group of Formula A is connected to said fused polycyclic ring system. In these compounds, the polycyclic ring system is optionally further substituted with one or more groups selected from C₁₋₄ alkyl, phenyl, C₁₋₄ alkoxy, halo, COOR*, —OH, CN, and —NR*₂, where each R* is independently selected from H and C₁₋₃ alkyl, where each C₁₋₃ alkyl is optionally substituted with one to three groups selected from —OH, —COOH, —NH₂, and C₁₋₂ alkyl. Moreover, the C₁-C₃ alkyl group that is substituted with at least a carboxyl group and an amino group can optionally be further substituted by a C₁-C₂ group that is optionally substituted with one to three groups selected from —OH, —COOH, —NH₂, and C₁₋₂ alkyl.

In some of these embodiments within embodiment 55, the polycyclic compound comprises a polycyclic aromatic or partially aromatic ring system comprising at least 10 fused 6-membered rings, wherein said polycyclic aromatic or partially aromatic ring system is fused to at least one subunit of Formula B:

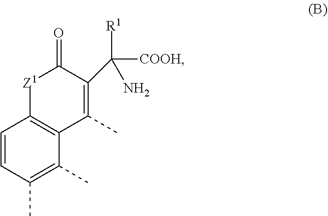

wherein:
R¹ is H or a C₁-C₃ alkyl optionally substituted with one or two groups selected from halo, —OH, —OMe, —NH₂, —SMe, —COOH, and —CONH₂,
Z¹ is NR² or C(R²)₂, where each R² is independently selected from H and C₁-C₃ alkyl, and wherein the dashed bonds indicate where said Formula B is fused to said polycyclic aromatic or partially aromatic ring system. In these compounds, the polycyclic ring system is optionally further substituted with one or more groups selected from C₁₋₄ alkyl, phenyl, C₁₋₄ alkoxy, halo, COOR*, —OH, CN, and —NR*₂, where each R* is independently selected from H and C₁₋₃ alkyl, where each C₁₋₃ alkyl is optionally substituted with one to three groups selected from —OH, —COOH, —NH₂, and C₁₋₂ alkyl. Moreover, the C₁-C₃ alkyl group that is substituted with at least a carboxyl group and an amino group can optionally be further substituted by a C₁-C₂ group that is optionally substituted with one to three groups selected from —OH, —COOH, —NH₂, and C₁₋₂ alkyl.
   56. The method of embodiment 55, wherein the method comprises solvothermal synthesis using at least one precursor comprising an alpha-amino carboxylic acid compound or an alpha-hydroxy carboxylic acid compound, and at least another precursor comprising a plurality of 6-member aromatic rings.
   57. The method of embodiment 56, wherein the precursor comprising an alpha-hydroxy carboxylic acid compound is citric acid (CA).
   58. The method of embodiment 56 or 57, wherein the precursor comprising a plurality of 6-membered aromatic rings is 1,4,5,8-tetraminoanthraquinone (TAAQ), 1,4-diaminoanthraquinone (1,4-DAAQ), or 1,5-diaminoanthraquinone (1,5-DAAQ).
   59. The method of embodiment 55, wherein the method comprises solvothermal synthesis using at least one precursor comprising phenylalanine or a phenylalanine analog, and a C₁-C₅ alcohol.
   60. The method of embodiment 59, wherein the precursor comprising phenylalanine or a phenylalanine analog is Phe.
   61. The method of embodiment 59 or 60, wherein the a C₁-C₈ alcohol is ethanol.
   62. The method of any of embodiments 55-61, which method comprises dissolving or dispersing the precursor molecules in a solvent to form a solution or a mixture and heating the solution or mixture at a temperature from about 100° C. to about 300° C. for a time from about 10 minutes to about 72 hours, e.g., about 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 20 hours, 30 hours, 40 hours, 50 hours, 60 hours, 70 hours, 72 hours, or any subrange thereof. In some embodiments, the temperature is between about 100° C., 150° C., 200° C., 250° C., 300° C., or any subrange thereof.
   63. The method of embodiment 62, wherein the solvent is water, a C₁₋₁₀ alcohol, e.g., ethanol, an amide, e.g., formamide, N,N-dimethyl formamide, dimethylacetamide, or N-methylpyrrolidone, a ketone, e.g., acetone or 2-butanone, or a sulfoxide, e.g., dimethylsulfoxide.
   64. The method of any of embodiments 55-63, which further comprises isolating or purifying a carbon quantum dot. In some of these embodiments, the carbon quantum dot fluoresces in the near-IR region. Optionally, the CQDs have an average particle size between about 1 nm and 5 nm, e.g., about 1 nm, 2 nm, 3 nm, 4 nm, or 5 nm, or any subrange thereof.
   65. The method of embodiment 64, wherein the carbon quantum dot is isolated or purified via filtration, centrifugation, chromatography, e.g., column chromatography, gel electrophoresis, or dialysis.
   66. The method of any of embodiments 55-65, which comprises:
1) mixing one precursor comprising an alpha-amino carboxylic acid compound or an alpha-hydroxy carboxylic acid compound, e.g., citric acid (CA), and another precursor comprising a plurality of 6-membered aromatic rings, e.g., TAAQ, 1,4-DAAQ or 1,5-DAAQ, in water to form a mixture;
2) heating the mixture at about 180° C. for about 2 hours;
3) cooling the mixture to a lower temperature, e.g., room temperature; and 4) isolating or purifying a carbon quantum dot from the mixture, e.g., via chromatography, solvent-solvent extraction, and/or crystallization.
   67. The method of embodiment 65 or 66, which further comprises removing the solvent, e.g., via drying under vacuum, to obtain an isolated or a purified carbon quantum dot.
   68. A carbon quantum dot that is prepared by a method of any of embodiments 55-67.
   69. The carbon quantum dot of any of embodiments 1-54 and 68, which further comprises a releasable cargo, e.g., the carbon quantum dot is covalently or non-covalently linked with a releasable cargo. In some of these embodiments, the cargo is a therapeutic agent, a chemotherapeutic agent, or a labeling agent. In some embodiments, the cargo is an aromatic chemotherapy drug, such as topotecan (TPTC), doxorubicin (DOX), or hydroxycamptothecin (HCPT).

In some of these embodiments, the quantum carbon dot is conjugated with a therapeutic agent, optionally by a releasable linker. Any suitable therapeutic agent can be used in these compositions and conjugates. For example, the therapeutic agent can be a small molecule therapeutic agent, a large molecule therapeutic agent, or a combination or complex thereof. Exemplary large molecule therapeutic agent can be a peptide, a polypeptide, a protein, e.g., a recombinant protein, an antibody, an antibody fragment or derivative, a polynucleotide or nucleic acid, e.g., a DNA, RNA, PNA, an anti-sense oligonucleotide or polynucleotide, or a RNAi oligonucleotide or polynucleotide, a lipid, or a sugar or carbohydrate, or a combination or complex thereof.

70. The carbon quantum dot of embodiment 69, wherein the release of the releasable cargo is triggered by a contact between the carbon quantum dot and a target cell, tissue, organ or subject, or by an enzymatic cleavage, or by a change of a physical and/or chemical parameter surrounding the carbon quantum dot.
71. The carbon quantum dot of embodiment 69 or 70, wherein the releasable cargo is a therapeutic agent, a prophylactic agent, a diagnostic agent, a marker agent or label, a prognostic agent, an imaging agent, or a combination thereof.
72. The carbon quantum dot of embodiment 71, wherein the releasable cargo is a therapeutic agent. Suitable therapeutic agents include topotecan (TPTC), doxorubicin (DOX), or hydroxycamptothecin (HCPT).
73. The carbon quantum dot of embodiment 72, wherein the therapeutic agent comprises a small molecule such as an organic compound having a molecular weight between 200 and 2,000, a large molecule, e.g., a polypeptide or polynucleotide, a cell therapy agent, a conjugate, or a combination thereof.
74. The carbon quantum dot of embodiment 72 or 73, wherein the therapeutic agent comprises an anti-tumor or anti-cancer agent.
75. The carbon quantum dot of embodiment 74, wherein the anti-cancer agent is a chemotherapy drug, e.g., topotecan hydrochloride (TPTC), doxorubicin (DOX), or hydroxycamptothecin (HCPT), or a kinase inhibitor.
76. The carbon quantum dot of any of embodiments 69-75, the therapeutic agent is configured for treating a cancer or tumor.
77. The carbon quantum dot of embodiment 76, wherein the therapeutic agent is configured for treating a tumor or cancer selected from the group consisting of sarcoma, epidermoid cancer, fibrosarcoma, cervical cancer, gastric carcinoma, skin cancer, leukemia, lymphoma, lung cancer, non-small cell lung cancer, colon cancer, melanoma, ovarian cancer, renal cancer, prostate cancer, breast cancer, liver cancer, head and neck cancers, pancreatic cancer, bladder cancer, esophagus cancer, tongue cancer, and cutaneous malignant melanoma.
78. A composition, which comprises the carbon quantum dot of any of embodiments 1-54 and 68, which is optionally a pharmaceutical composition comprising the carbon quantum dot of any of embodiments 1-54 and 68 admixed with at least one pharmaceutically acceptable carrier or excipient.
79. The composition of embodiment 78, which is configured for use as a therapeutic, a prophylactic, a diagnostic, a sensing, a marking, a prognostic, an imaging application, or a combination thereof, such as by having the carbon quantum dot linked or releasably linked to a therapeutic agent such as a therapeutic agent for treating a cancer, such as topotecan hydrochloride (TPTC), doxorubicin (DOX), or hydroxycamptothecin (HCPT), or a kinase inhibitor.
80. A kit, which comprises the carbon quantum dot of any of embodiments 1-54 and 68-77, or the composition of embodiment 78 or 79, and an instruction for using the carbon quantum dot or the composition.
81. A method for sensing, marking or imaging a target cell, tissue or organ in a subject, which method comprises:
a) administering, to a subject in need, an effective amount of a carbon quantum dot of any of embodiments 1-54 and 68-77, or a composition of embodiment 78 or 79; and
b) assessing said carbon quantum dot for sensing, marking or imaging a target cell, tissue or organ in said subject.

In these embodiments, the step of assessing said carbon quantum dot can comprise detecting near-IR fluorescence produced by the carbon quantum dot.

82. The method of embodiment 81, wherein the carbon quantum dot does not comprise a releasable cargo.
83. The method of embodiment 81, wherein the carbon quantum dot comprises a releasable cargo.
84. The method of any of embodiments 81-83, which is used for sensing, marking or imaging a target cell, tissue or organ in a subject.
85. The method of any of embodiments 81-83, which is used for sensing, marking or imaging an abnormal or diseased cell, tissue or organ in a subject.
86. The method of embodiment 85, which is used for sensing, marking or imaging a tumor or cancer cell, tissue or organ in a subject.
87. The method of embodiment 85, which is used for sensing, marking or imaging a tumor in a subject.
88. The method of any of embodiments 85-87, which is used for diagnosis, prognosis, stratification, risk assessment, or treatment monitoring of a disease or disorder in a subject.
89. The method of embodiment 88, wherein the disease or disorder is a tumor or cancer selected from the group consisting of sarcoma, epidermoid cancer, fibrosarcoma, cervical cancer, gastric carcinoma, skin cancer, leukemia, lymphoma, lung cancer, non-small cell lung cancer, colon cancer, melanoma, ovarian cancer, renal cancer, prostate cancer, breast cancer, liver cancer, head and neck cancers, pancreatic cancer, bladder cancer, esophagus cancer, tongue cancer, and cutaneous malignant melanoma.
90. The method of any of embodiments 85-87, which is used for assisting or guiding therapy or treatment of a disease or disorder in a subject.
91. The method of embodiment 90, wherein the therapy or treatment comprises a procedure or surgery on a subject.
92. The method of embodiment 91, which is used for assisting or guiding a procedure or surgery on a tumor or cancer in a subject.
93. The method of embodiment 91, which is used for assisting or guiding a radiation treatment of a subject.
94. Use of an effective amount of a carbon quantum dot of any of embodiments 1-54 and 68-77, or a composition of embodiment 78 or 79, for the manufacture of a composition for sensing, marking or imaging a target cell, tissue or organ in a subject in need.
95. A pharmaceutical composition, which comprises an effective amount of a carbon quantum dot of any of embodiments 1-77, e.g., embodiments 72-77, or a composition of embodiment 78 or 79, and a pharmaceutically acceptable carrier or excipient. In some embodiments, the pharmaceutical composition further comprises a therapeutic agent for treating a cancer, such as topotecan hydrochloride (TPTC), doxorubicin (DOX), or hydroxycamptothecin (HCPT), or a kinase inhibitor, wherein the therapeutic agent is optionally releasably linked to the carbon quantum dot.
96. A method for treating or preventing a disease or condition in a subject, which comprises administering to a subject in need thereof an effective amount of a carbon quantum dot of any of embodiments 1-77, e.g., embodiments 72-77, a composition of embodiment 78 or 79, or a pharmaceutical composition of embodiment 95.
97. The method of embodiment 96, which is used for treating or preventing a tumor or cancer in a subject.
98. The method of embodiment 96, which is used for treating a tumor in a subject.
99. The method of any of embodiments 96-98, wherein the carbon quantum dot, composition or pharmaceutical composition is administered via an oral, nasal, inhalational, parental, intravenous, intraperitoneal, subcutaneous, intramuscular, intradermal, topical, or rectal route.
100. The method of any of embodiments 81-93 and 96-99, wherein the subject is a mammal.
101. The method of embodiment 100, wherein the mammal is a non-human mammal.
102. The method of embodiment 100, wherein the mammal is a human.
103. Use of an effective amount of a carbon quantum dot of any of embodiments 1-54 and 68-77, or a composition of embodiment 78 or 79 for the manufacture of a medicament for treating or preventing a disease or condition in a subject in need, or for detecting the presence or absence of a cancerous cell or tissue in a subject in need thereof.
104. The use of embodiment 103, wherein the medicament is used for treating or preventing a tumor or cancer in a subject.
105. The use of embodiment 103, wherein the medicament is used for treating a tumor in a subject.
106. The composition of claim 78 or 79, wherein the carbon quantum dot is attached to or immobilized on a solid surface or support.
107. The composition of claim 106, which is configured for detecting and/or isolating a target cell from a sample.
108. The composition of claim 107, wherein the sample is a biological sample, e.g., a blood or urine sample.
109. The composition of claim 107 or 108, which is configured for detecting and/or isolating a tumor or cancer cell from a sample.
110. The composition of claim 109, which is configured for detecting and/or isolating a circulating tumor or cancer cell from a biological sample, e.g., a blood or urine sample.
111. The composition of any of claims 106-110, wherein the solid surface or support is part of a container, a tube, a plate, a microtiter plate, e.g., a well of a microtiter plate, a disk, a chip, a membrane or a collection and/or assay device.
112. The composition of any of claims 106-110, wherein the solid surface or support is part of a device used in an assay selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, u-capture assay, inhibition assay and avidity assay.
113. A method for detecting and/or isolating a tumor or cancer cell from a sample, which method comprises contacting a sample containing or suspected of containing a tumor or cancer cell with a composition of any of claims 106-110, under suitable conditions, to allow binding of said tumor or cancer cell, if present in said sample, to said carbon quantum dot attached to or immobilized on said solid surface or support.
114. The composition of claim 113, which is used to detect and/or isolate a circulating tumor or cancer cell from a biological sample, e.g., a blood or urine sample.
115. The method of any of claims 81-90, which is used to detect and/or isolate a circulating tumor or cancer cell from a biological sample, e.g., a blood or urine sample.
116. A carbon quantum dot of any of embodiments 1-54 and 68-77, or a composition of embodiment 78 or 79, for use in therapy.
117. The carbon quantum dot of embodiment 116, wherein the use in therapy is use to treat or detect a cancer or a tumor.
118. The carbon quantum dot of embodiment 116, wherein the use in therapy is use to treat a tumor.
119. The composition of any of embodiments 106-110, wherein the solid surface or support is part of a container, a tube, a plate, a microtiter plate, e.g., a well of a microtiter plate, a disk, a chip, a membrane or a collection and/or assay device.
120. The composition of any of embodiments 106-110, wherein the solid surface or support is part of a device used in an assay selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, plasmon resonance assay, chemiluminescence assay, lateral flow immunoassay, u-capture assay, inhibition assay and avidity assay.
121. A method for detecting and/or isolating a tumor or cancer cell from a sample, which method comprises contacting a sample containing or suspected of containing a tumor or cancer cell with a composition of any of embodiments 106-110, under suitable conditions, to allow binding of said tumor or cancer cell, if present in said sample, to said carbon quantum dot attached to or immobilized on said solid surface or support.
122. The composition of embodiment 113, which is used to detect and/or isolate a circulating tumor or cancer cell from a biological sample, e.g., a blood or urine sample.
123. The method of any of embodiments 81-90, which is used to detect and/or isolate a circulating tumor or cancer cell from a biological sample, e.g., a blood or urine sample.

The following exemplary compounds as well as carbon quantum dots or particles that comprise these compounds are preferred embodiments of the invention, and methods of making and using these exemplary compounds are preferred embodiments of the invention.

### Exemplary Compounds

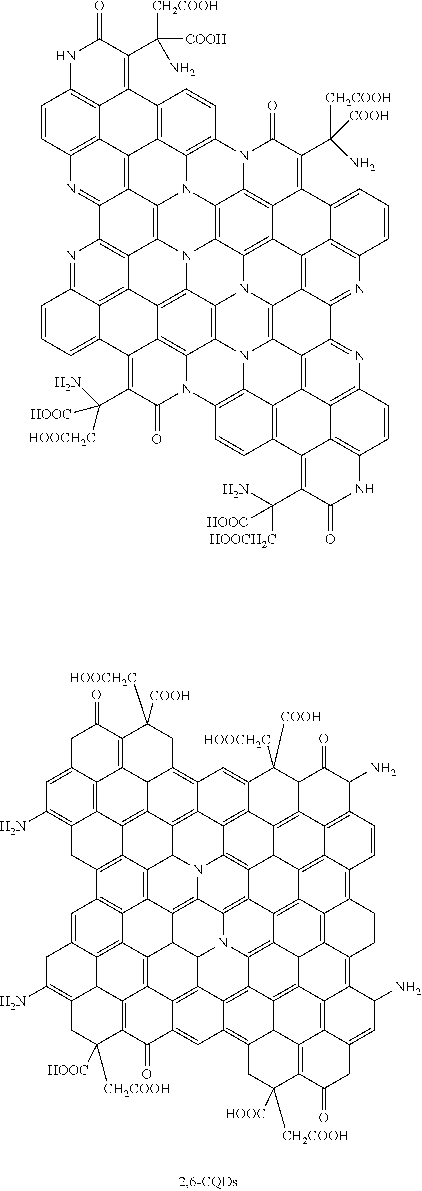

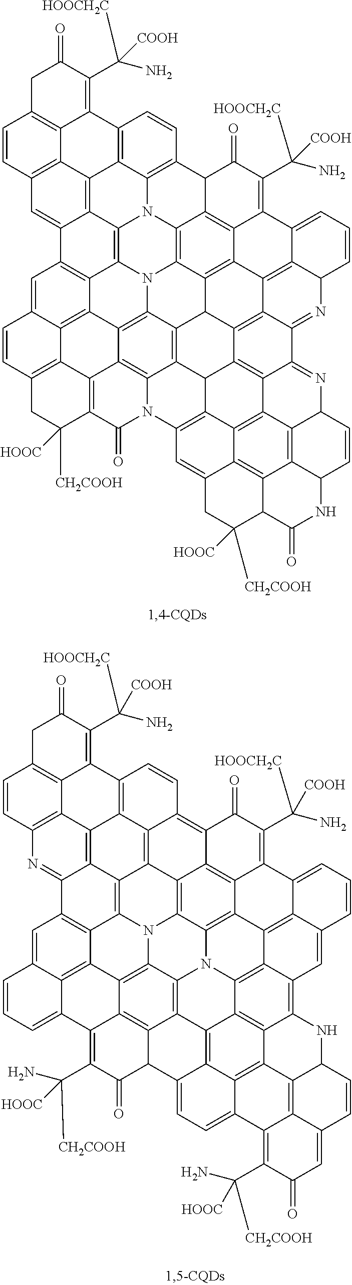

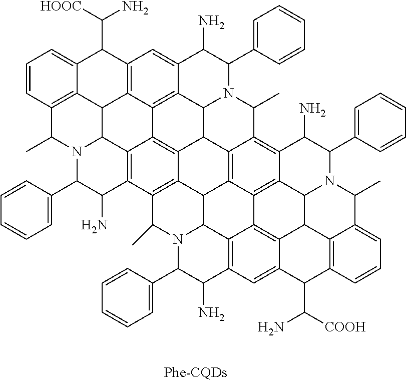 [SMILES: CC1c2cccc3c2C2c4c(cc5c6c4-c4c7c8c(c9c4C2N1C(c1ccccc1)C9N)C(C)N1C(c2ccccc2)C(N)c2cc4c9c%10c2C1C8c1c-%10c2c(c8c1C7C6N(C8C)C(c1ccccc1)C5N)C(N)C(c1ccccc1)N1C(C)c5cccc(c5C9C21)C4C(N)C(=O)O)C3C(N)C(=O)O]

The compounds, particles and compositions described herein can be used for any suitable purpose. In some embodiments, the compounds, particles and compositions described herein can be used in therapy, such as for treatment of a tumor that expresses LAT1. The invention also provides conjugates comprising the compounds and particles described herein that are also suitable for use in therapy. In some embodiments, therapy refers to treatment of a tumor, especially one that expresses LAT1.

In another aspect, the compounds are useful to image a cell, tissue, organ, or tumor, typically one that expresses LAT1. The invention thus includes methods for using the compounds, particles and compositions as imaging or labeling agents, and include conjugates comprising the compounds and particles described herein that are suitable for use in these methods.

In another aspect, the compounds of the invention include conjugates of carbon quantum dots with a cargo, which is usually a therapeutic agent useful to treat or image a cell, tissue, organ or tumor that expresses LAT1. The cargo is releasable connected to the CQD; in some embodiments, it is covalently connected to the CQD, optionally via an amide or ester linkage between the cargo and an amino or carboxyl group of the CQD. In other embodiments, the cargo is physically adsorbed to a particle(s) or CQDs of the invention, and release of the cargo inside a cell occurs passively. This method of carrying a cargo is particularly effective for a polycyclic aromatic cargo, or cargo compounds having strong pi stacking ability such as topotecan or irinotecan, doxorubicins, and daunomycins.

Herein are disclosed polycyclic compounds and particles comprising these compounds, including certain carbon quantum dots, that selectively bind to or internalize inside tumorous or cancerous cells. Without being bound by theory, it is believed that their selectivity derives from their recognition by large amino acid transporters

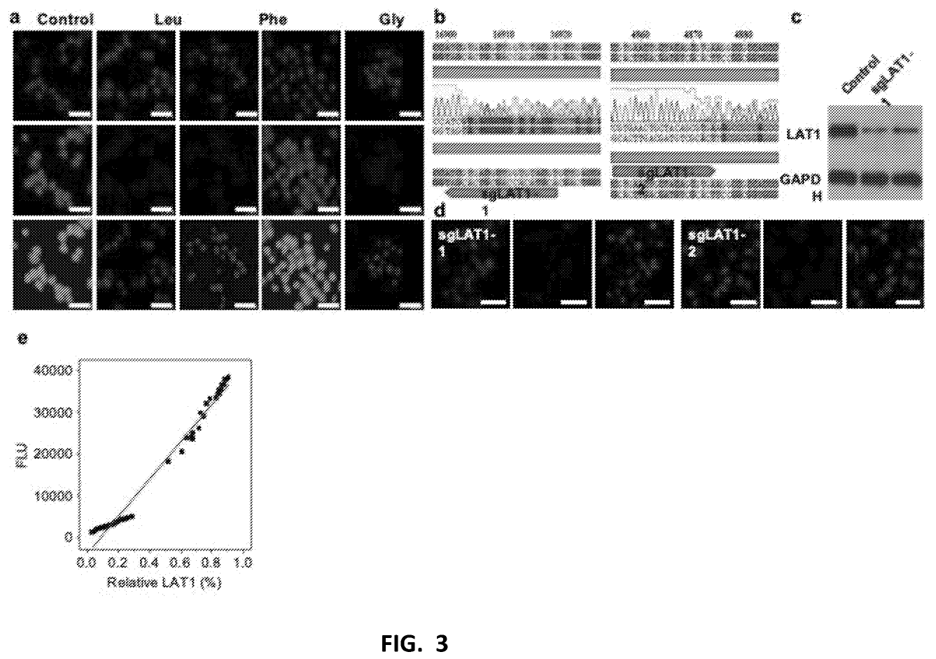

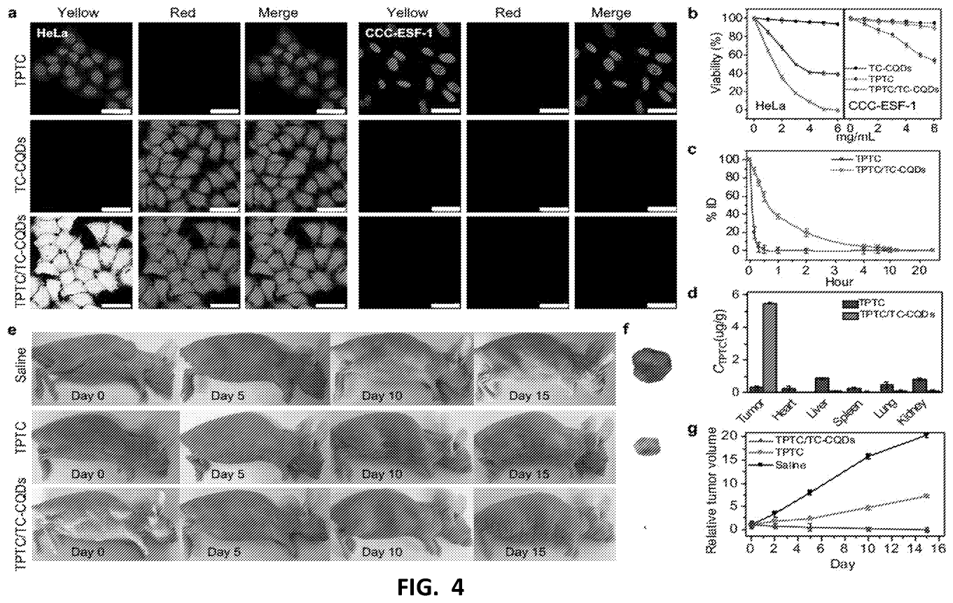

LAAM CQDs and demonstrated that LAAM CQDs allow imaging of and drug delivery to tumors of different origin and location with unprecedented specificity and efficiency (FIG. 2, 4). Through a combination of genetic and pharmacological approaches we found that the tumor-specific interaction is mediated through LAT1 (FIG. 3). LAAM CQDs have three unique and significant advantages over prior imaging and delivery reagents:

First, LAAM CQDs, like previously reported CQDs, bear intrinsic optical properties for imaging and thus can be used for tumor imaging without further conjugation of imaging probes. We characterized LAAM TC-CQDs, one of the preferred LAAM CQDs, and found it allows for NIR FL/PA dual-mode imaging that is optimal for deep tissue imaging. Due to its high specificity to tumors, LAAM TC-CQDs have great potential to be utilized for tumor imaging as well as for image-guided surgery.

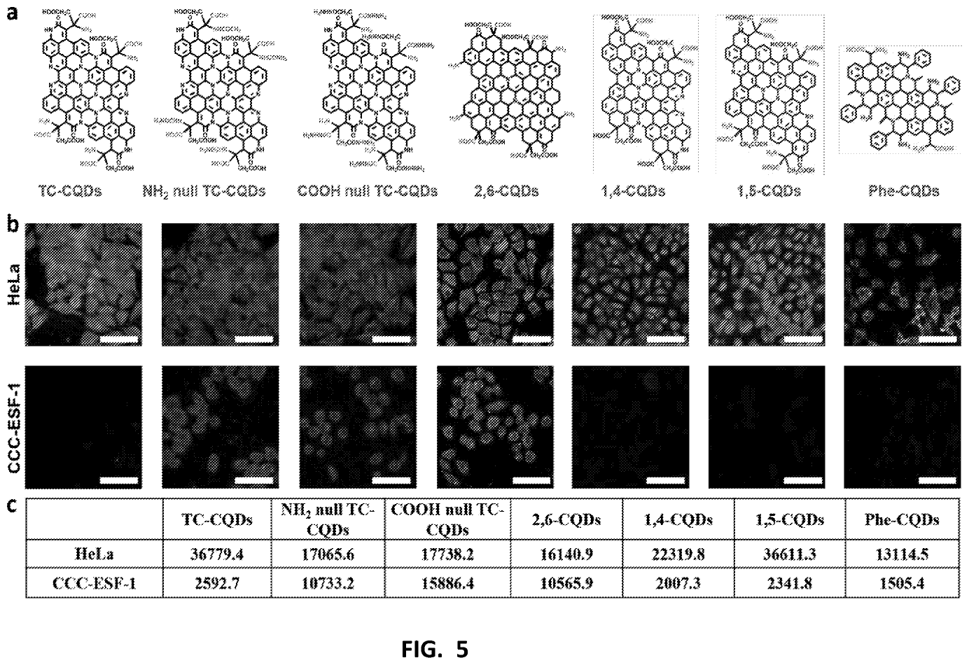

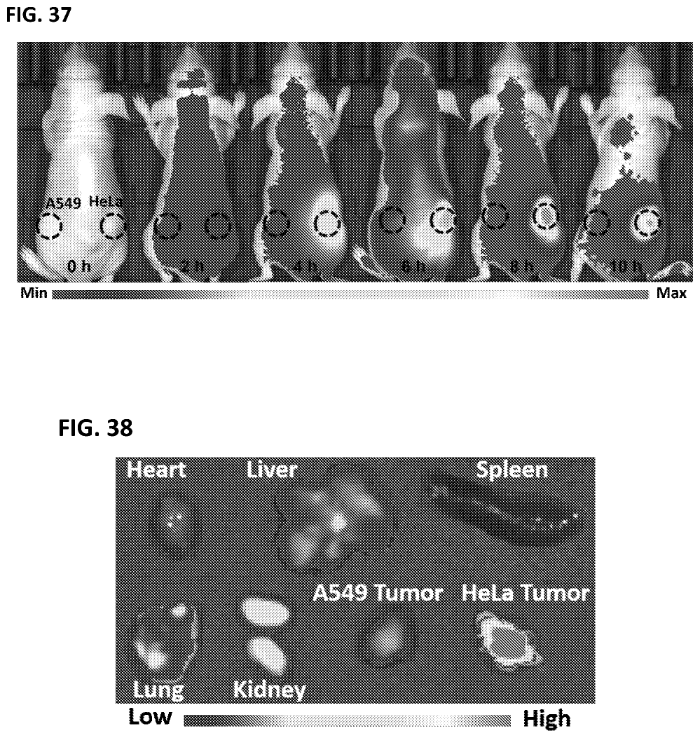

Second, LAAM TC-CQDs allow targeted drug delivery to tumors with a high degree of specificity, which cannot be achieved via the traditional approaches through conjugation of tumor-targeting ligands. A recent analysis showed that, despite tremendous efforts, the traditional approaches have failed to significantly improve the tumor-targeting efficiency. In consistence with the finding, we found that conjugation of ligands enhanced the delivery of CQDs to some tumors, but not others, and cannot significantly reduce their nonspecific accumulation in normal tissues (FIGS. 37, 38). The ligand-conjugation approach has been previously tested for LAT1-mediated drug delivery to tumors through conjugation of aspartate 13. It was found that LAT1-targeting improved the accumulation of DOX in tumors by 3-6-fold. However, a comparable amount of DOX was also found in the liver. In contrast, we found that delivery via LAAM TC-CQDs enhanced the accumulation of TPTC in tumors by 16.7-fold, while the accumulation of TPTC in the liver was reduced to a barely detectable level (1.8% of that in the tumors) (FIG. 4d). The observed difference between the two studies may be because that LAAM TC-CQDs and aspartate as drug carriers have different affinities with LAT1. LAAM TC-CQDs bear 4 pairs of α-carboxyl and amino groups. We found that the specificity of LAAM CQDs is correlated with the number of paired α-carboxyl and amino groups on the core (FIG. 5). It appears that the local high density of α-carboxyl and amino groups triggers a multivalent, strong interaction with LAT1. As a result, delivery via LAAM TC-CQDs significantly enhance the penetration of TPTC into tumor cells. Because most normal tissues don't express LAT1, LAAM TC-CQDs do not readily penetrate normal cells and are preferentially eliminated from the circulation system (FIG. 2b) in the absence of active transporter. Different from LAAM CQDs, aspartate bears only one pair of α-carboxyl and amino group. Without multivalency, the interaction between aspartate-conjugated DOX with LAT1 could be weak. Consequently, aspartate-conjugated DOX has a low affinity for tumor cells and can non-specifically penetrate normal cells, leading to the accumulation in normal organs¹³.

Taken together, LAAM CQDs have an ideal set of properties for tumor-specific imaging and drug delivery. Due to their ability to selectively target tumors regardless of their origin and location, their minimal accumulation in most normal tissues, their minimal toxicity, their intrinsic imaging capacity, and their ability to load and deliver chemotherapy drugs, LAAM CQDs have remarkable potential for translation into clinical applications such as imaging and drug delivery to various tumors.

### Synthesis and Characterization of LAAM TC-CQDs

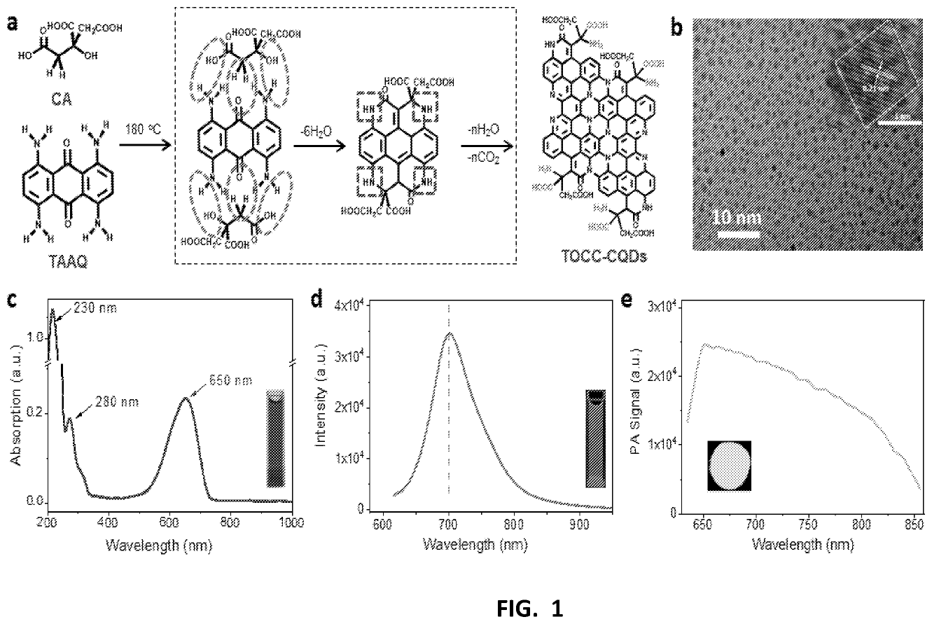

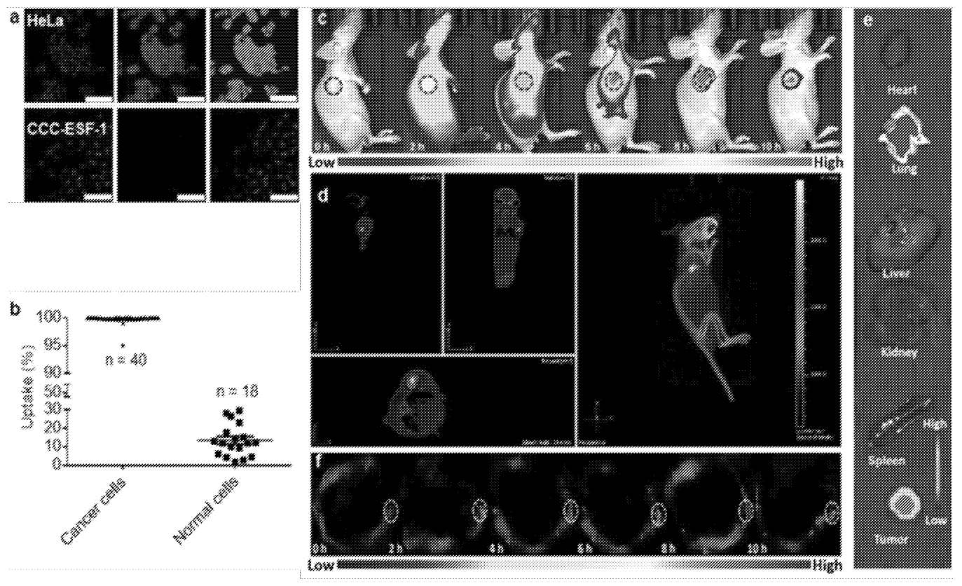

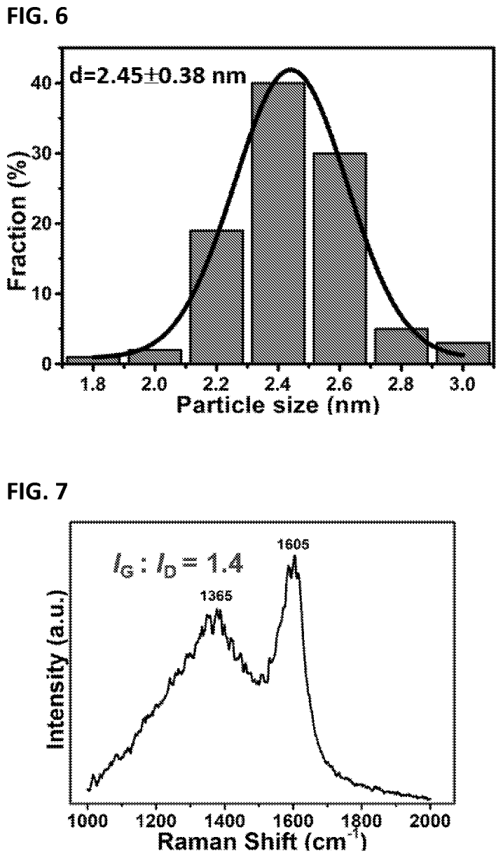

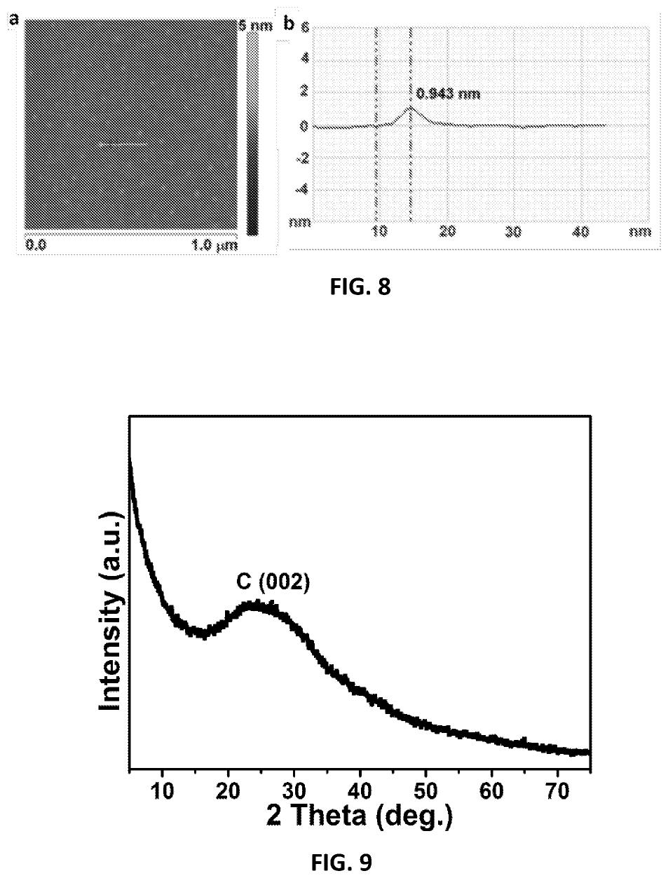

LAAM TC-CQDs were synthesized by mixing 1,4,5,8-tetraminoanthraquinone (TAAQ) with citric acid (CA) in aqueous solution, following with hydrothermal treatment at 180° C. for 2 h (FIG. 1a). The reactant was purified using silica gel column chromatography, resulting in a clear blue solution. Analysis by transmission electron microscopy (TEM) shows that LAAM TC-CQDs are well-dispersed with an average diameter of 2.45 nm (FIG. 1b and FIG. 6). High-resolution TEM (HRTEM) analysis reveals a well-resolved crystal lattice with spacing of 0.21 nm (FIG. 1b inset), which is consistent with the (100) lattice fringes of graphene¹⁶. The high degree of crystallinity was confirmed by Raman spectrum analysis, in which the crystalline G band at 1605 cm⁻¹ is stronger than the disordered D band at 1365 cm⁻¹, with a G to D intensity ratio (I_{G}/I_{D}) of 1.4 (FIG. 7). Atomic force microscope (AFM) analysis identify a typical topographic height of 0.943 nm (FIG. 8), suggesting that most of LAAM TC-CQDs consist of 2-3 graphene layers. X-ray powder diffraction (XRD) pattern reveals a broad (002) peak centered at ˜26° (FIG. 9), confirming the graphene structure of LAAM TC-CQDs.

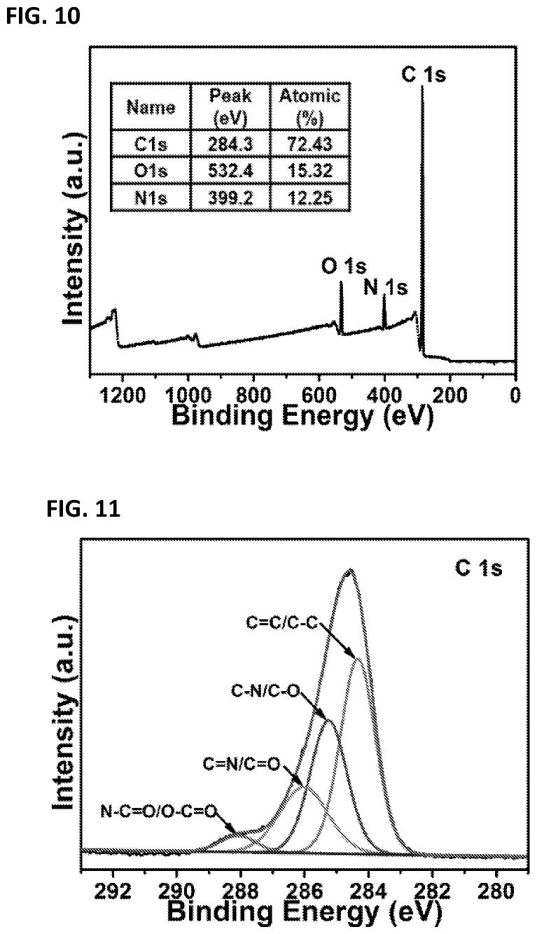

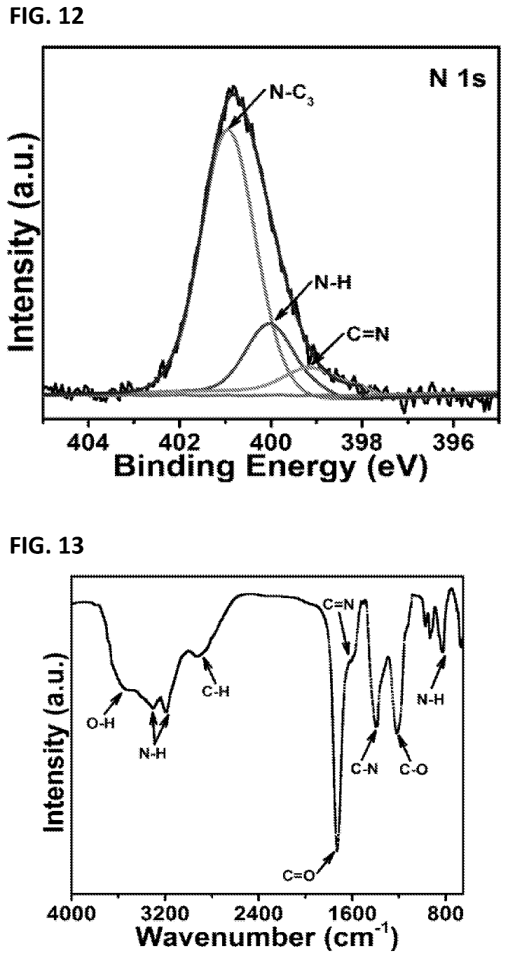

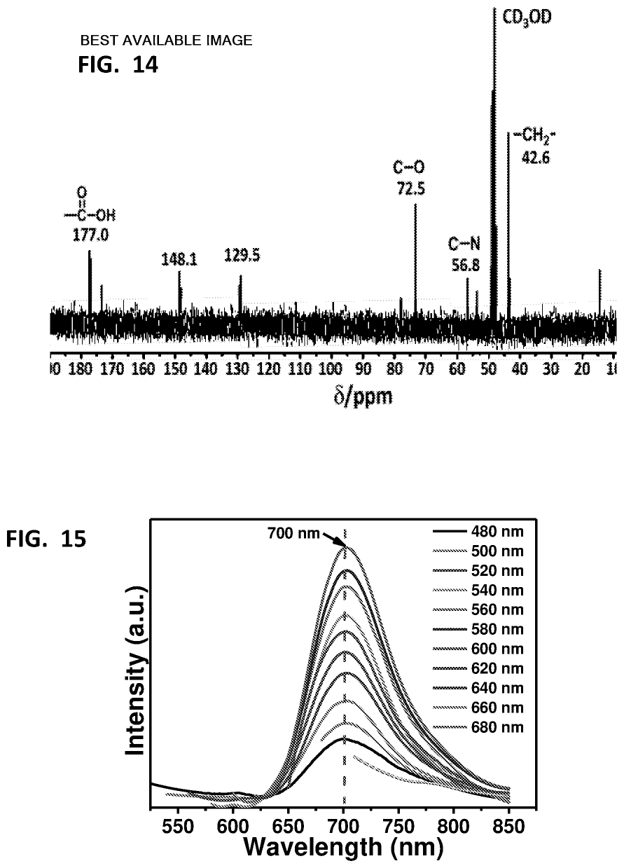

The chemical composition and surface functional groups of LAAM TC-CQDs were characterized by X-ray photoelectron spectroscopy (XPS), Fourier transform infrared (FT-IR) and ¹³C— nuclear magnetic resonance spectrum (NMR). XPS survey spectrum analysis suggests the presence of C, N and O with atomic percentages of 72.43%, 12.25% and 15.32% in LAAM TC-CQDs, respectively (FIG. 10). The deconvoluted C1s spectrum reveals that there are three different types of carbon atoms, including graphitic/aliphatic (C═C/C—C), oxygenated (C—O, C═O, and O—C═O), and N-containing (C—N, C═N, and N—C═O) (FIG. 11). The deconvoluted N1s spectrum shows three peaks at 399.0, 399.9 and 400.8 eV (FIG. 12), which correspond to C—N, N—H and C═N, respectively. FT-IR spectrum analysis identify stretching vibrations for N—H (3298 and 3190 cm⁻¹), C═O (1720 cm⁻¹), C═N (1624 cm⁻¹), C—N(1387 cm⁻¹) and C—O (1158 cm⁻¹) bonds (FIG. 13), suggesting the formation of polyaromatic structures and the presence of free carboxyl and amino groups at the edge of LAAM TC-CQDs. In the ¹³C-NMR spectrum, peaks between 120.0 and 140.0 ppm can be ascribed to aromatic functionalities and conjugated double bonds (C═C and C═N), and peaks at 173.6, 170.2 and 53.3 ppm are associated with O—C═O, N—C═O and C—N, respectively (FIG. 14). Collectively, the obtained structural information suggests that LAAM TC-CQDs might be formed through two steps. In the first step under the hydrothermal condition, one molecule of TAAQ reacts with two molecules of CA, resulting a rigid carbon skeleton consisting of three benzene rings fused with four pyridine rings. Subsequently, the skeletons as building blocks undergo dehydration and decarboxylation, leading to the formation of LAAM TC-CQDs with high N doping in the large rigid π-conjugated structure and free α-carboxyl and amino groups at the edge (FIG. 1a).

The UV-vis absorption spectrum of LAAM TC-CQDs exhibits a strong characteristic absorption band centered at about 650 nm (FIG. 1c) except two typical absorption peaks of CQDs at 230 and 280 nm assigned to the π-π* transition of the aromatic C═C bond and n-π* transitions of the aromatic sp² system containing C═O and C═N bonds, respectively^{14,17-19}. A detailed fluorescence (FL) characterization was carried out with different excitation wavelengths (FIG. 1d, FIG. 15). LAAM TC-CQDs in aqueous solution exhibit a near-infrared fluorescence (NIR FL) emission peak at 700 nm, and the FL emission wavelength is nearly excitation-independent. The absolute FL quantum yield of LAAM TC-CQDs was determined to be 6.8% using a spectrometer attached to an integrating sphere.

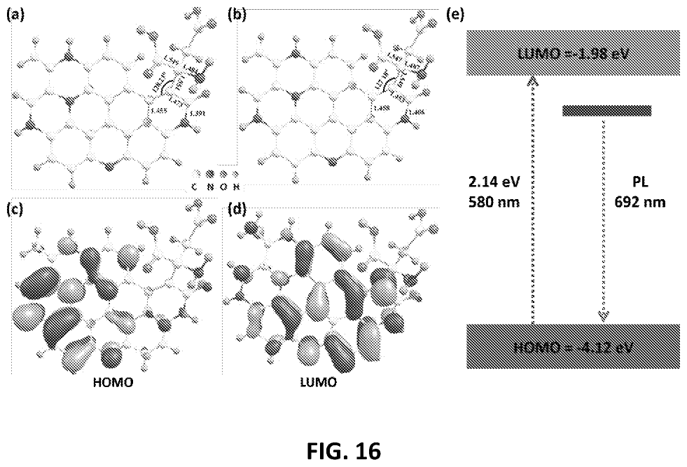

Density functional theory (DFT) simulations using the B3LYP/6-31G(d,p) basis set confirmed that LAAM TC-CQDs bear the local structure for generation of NIR FL. The optimized cartesian coordinates and main geometric parameters for the ground and excited structures of LAAM TC-CQDs are shown in FIG. 16a,b and Table 1. The electron delocalization molecular orbital (MO) diagrams of one FL unit obtained from theoretical calculations showed that the energy gap (Eg) between the highest occupied molecular orbital (HOMO) level and the lowest unoccupied molecular orbital (LUMO) level is 2.14 eV (FIG. 16c-e). The simulation predicted that the excitation wavelength is at 580 nm with an oscillator strength of 0.1092, and the corresponding fluorescence wavelength is 692 nm with an oscillator strength of 0.1003; these values are close to the experimentally measured excitation/emission wavelengths (λex=600 nm, λem=700 nm, FIG. 15).

TABLE 1
Optimized cartesian coordinates (Å) of the FL unit for ground state
and excited state

| Species | Cartesian coordinates | | | |
|---|---|---|---|---|
| Ground state | C | −2.764883000 | 0.834352000 | −0.192789000 |
| | C | −3.524862000 | 2.024114000 | −0.611560000 |
| | N | −2.795185000 | 3.203204000 | −0.716309000 |
| | C | −1.443244000 | 3.315502000 | −0.546818000 |
| | C | −0.705703000 | 2.140280000 | −0.407935000 |
| | C | −1.374222000 | 0.849602000 | −0.344996000 |
| | C | −0.503083000 | −0.334604000 | −0.441505000 |
| | C | −0.964823000 | −1.593053000 | −0.850074000 |
| | N | −4.435073000 | 0.475433000 | 1.515857000 |
| | C | −3.643771000 | −0.246351000 | 0.488761000 |
| | C | 0.704029000 | 2.253794000 | −0.214384000 |
| | C | 1.485176000 | 1.102994000 | −0.075788000 |
| | C | 0.897709000 | −0.199868000 | −0.216694000 |
| | C | −0.806906000 | 4.595263000 | −0.520000000 |
| | C | 0.539384000 | 4.699491000 | −0.312365000 |
| | C | 1.355558000 | 3.531184000 | −0.132299000 |
| | C | 1.760430000 | −1.315492000 | −0.242612000 |
| | C | 1.240699000 | −2.586030000 | −0.581203000 |
| | C | −0.112937000 | −2.692483000 | −0.912315000 |
| | N | 2.664121000 | 3.679891000 | 0.115455000 |
| | C | 3.424159000 | 2.561545000 | 0.263820000 |
| | C | 2.867532000 | 1.240616000 | 0.139644000 |
| | C | 3.686704000 | 0.089266000 | 0.244490000 |
| | N | 3.114950000 | −1.181344000 | 0.020096000 |
| | O | −4.728171000 | 2.057800000 | −0.905187000 |
| | C | −2.818500000 | −1.276894000 | 1.298608000 |
| | O | −2.064374000 | −0.968841000 | 2.200127000 |
| | O | −3.123401000 | −2.558243000 | 1.014396000 |
| | C | −4.610119000 | −0.903767000 | −0.528908000 |
| | C | −5.641988000 | −1.867416000 | 0.030445000 |
| | O | −6.115409000 | −1.827976000 | 1.149656000 |
| | O | −6.107453000 | −2.794918000 | −0.833167000 |
| | C | 4.806960000 | 2.664647000 | 0.547191000 |
| | C | 5.577756000 | 1.525411000 | 0.699077000 |
| | C | 5.033925000 | 0.233269000 | 0.556057000 |
| | N | 5.813173000 | −0.928569000 | 0.745103000 |
| | C | 5.304465000 | −2.132671000 | 0.280878000 |
| | C | 4.017952000 | −2.305362000 | −0.081603000 |
| | C | 3.485256000 | −3.584946000 | −0.582955000 |
| | N | 2.089904000 | −3.673081000 | −0.628934000 |
| | C | 4.241810000 | −4.638608000 | −0.952924000 |
| | H | −3.348599000 | 4.019865000 | −0.949647000 |
| | H | −1.989139000 | −1.723031000 | −1.166741000 |
| | H | −3.806913000 | 0.715190000 | 2.279626000 |
| | H | −5.137793000 | −0.167313000 | 1.880125000 |
| | H | −1.415727000 | 5.484842000 | −0.650519000 |
| | H | 1.024318000 | 5.669022000 | −0.270585000 |
| | H | −0.498757000 | −3.653171000 | −1.240063000 |
| | H | −2.628107000 | −3.121116000 | 1.635501000 |
| | H | −4.036520000 | −1.397551000 | −1.316577000 |
| | H | −5.156435000 | −0.077644000 | −0.997561000 |
| | H | −5.657760000 | −2.734083000 | −1.689846000 |
| | H | 5.246458000 | 3.650833000 | 0.647271000 |
| | H | 6.635524000 | 1.615332000 | 0.929953000 |
| | H | 6.816584000 | −0.817973000 | 0.711202000 |
| | H | 6.004102000 | −2.955371000 | 0.248117000 |
| | H | 1.709493000 | −4.533072000 | −0.998899000 |
| | H | 3.777994000 | −5.567247000 | −1.266632000 |
| | H | 5.321743000 | −4.601546000 | −0.945665000 |
| Excited state | C | −2.788355000 | 0.818576000 | −0.219107000 |
| | C | −3.538408000 | 1.991830000 | −0.633312000 |
| | N | −2.797996000 | 3.175923000 | −0.798556000 |
| | C | −1.454194000 | 3.305119000 | −0.559065000 |
| | C | −0.717210000 | 2.137481000 | −0.378081000 |
| | C | −1.365736000 | 0.832137000 | −0.354764000 |
| | C | −0.490392000 | −0.333510000 | −0.496711000 |
| | C | −0.924838000 | −1.592907000 | −0.940951000 |
| | N | −4.406769000 | 0.450758000 | 1.566267000 |
| | C | −3.650140000 | −0.251536000 | 0.495671000 |
| | C | 0.678046000 | 2.266159000 | −0.159215000 |
| | C | 1.474728000 | 1.108985000 | −0.048396000 |
| | C | 0.909285000 | −0.204857000 | −0.256821000 |
| | C | −0.830299000 | 4.594473000 | −0.498667000 |
| | C | 0.507275000 | 4.710340000 | −0.245407000 |
| | C | 1.317827000 | 3.535616000 | −0.062075000 |
| | C | 1.773291000 | −1.309112000 | −0.317469000 |
| | C | 1.270650000 | −2.593389000 | −0.645387000 |
| | C | −0.076140000 | −2.700426000 | −0.992138000 |
| | N | 2.637056000 | 3.676427000 | 0.202274000 |
| | C | 3.394228000 | 2.566941000 | 0.321401000 |
| | C | 2.846244000 | 1.246870000 | 0.161931000 |
| | C | 3.686565000 | 0.104711000 | 0.202229000 |
| | N | 3.151626000 | −1.172766000 | −0.067138000 |
| | O | −4.761884000 | 2.066235000 | −0.865137000 |
| | C | −2.805275000 | −1.306635000 | 1.248843000 |
| | O | −2.047982000 | −1.035317000 | 2.160214000 |
| | O | −3.076252000 | −2.581089000 | 0.896435000 |
| | C | −4.659303000 | −0.890431000 | −0.492002000 |
| | C | −5.674884000 | −1.857063000 | 0.086170000 |
| | O | −6.048321000 | −1.897153000 | 1.242697000 |
| | O | −6.245992000 | −2.702370000 | −0.802009000 |
| | C | 4.790886000 | 2.681388000 | 0.598630000 |
| | C | 5.592095000 | 1.561977000 | 0.681730000 |
| | C | 5.062230000 | 0.270802000 | 0.474891000 |
| | N | 5.850528000 | −0.856963000 | 0.521575000 |
| | C | 5.342237000 | −2.091855000 | 0.243766000 |
| | C | 4.015726000 | −2.279033000 | −0.088569000 |
| | C | 3.498924000 | −3.601313000 | −0.479064000 |
| | N | 2.114229000 | −3.681218000 | −0.637019000 |
| | C | 4.270294000 | −4.691525000 | −0.675008000 |
| | H | −3.358116000 | 3.987855000 | −1.025977000 |
| | H | −1.946658000 | −1.721714000 | −1.272086000 |
| | H | −3.734329000 | 0.726663000 | 2.278745000 |
| | H | −5.043405000 | −0.222568000 | 1.992681000 |
| | H | −1.444154000 | 5.478716000 | −0.643876000 |
| | H | 0.987086000 | 5.680897000 | −0.179546000 |
| | H | −0.464622000 | −3.662405000 | −1.312205000 |
| | H | −2.555289000 | −3.159375000 | 1.481114000 |
| | H | −4.118125000 | −1.378819000 | −1.306944000 |
| | H | −5.211465000 | −0.049772000 | −0.928692000 |
| | H | −5.871415000 | −2.579761000 | −1.687714000 |
| | H | 5.207102000 | 3.673074000 | 0.735281000 |
| | H | 6.652343000 | 1.659615000 | 0.893503000 |
| | H | 6.832405000 | −0.768720000 | 0.745917000 |
| | H | 6.035722000 | −2.915509000 | 0.292455000 |
| | H | 1.739440000 | −4.567205000 | −0.945012000 |
| | H | 3.818204000 | −5.639288000 | −0.943718000 |
| | H | 5.345848000 | −4.668145000 | −0.581660000 |

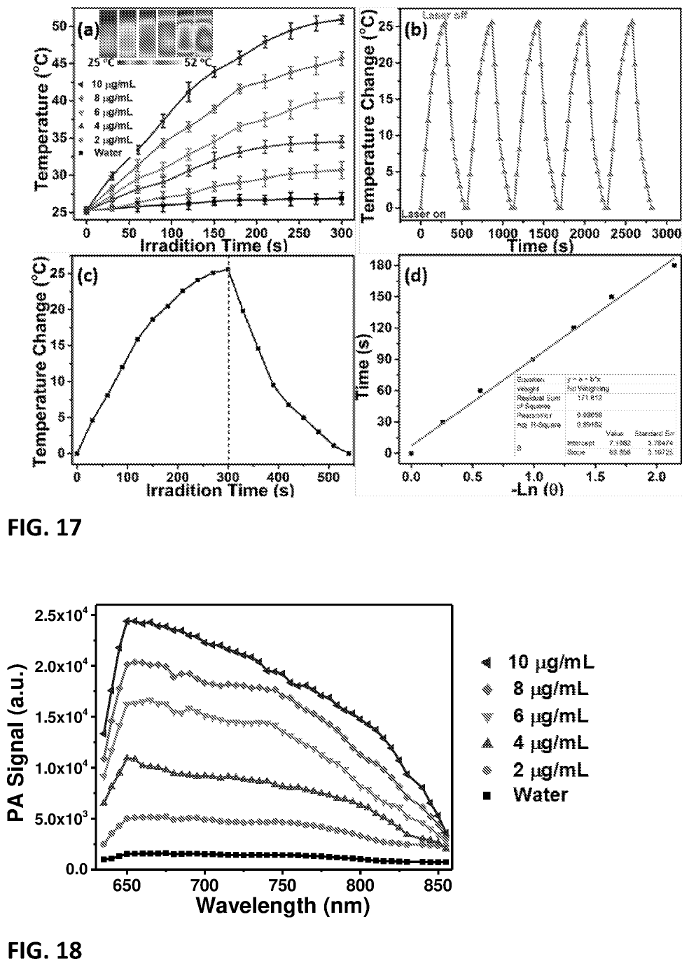

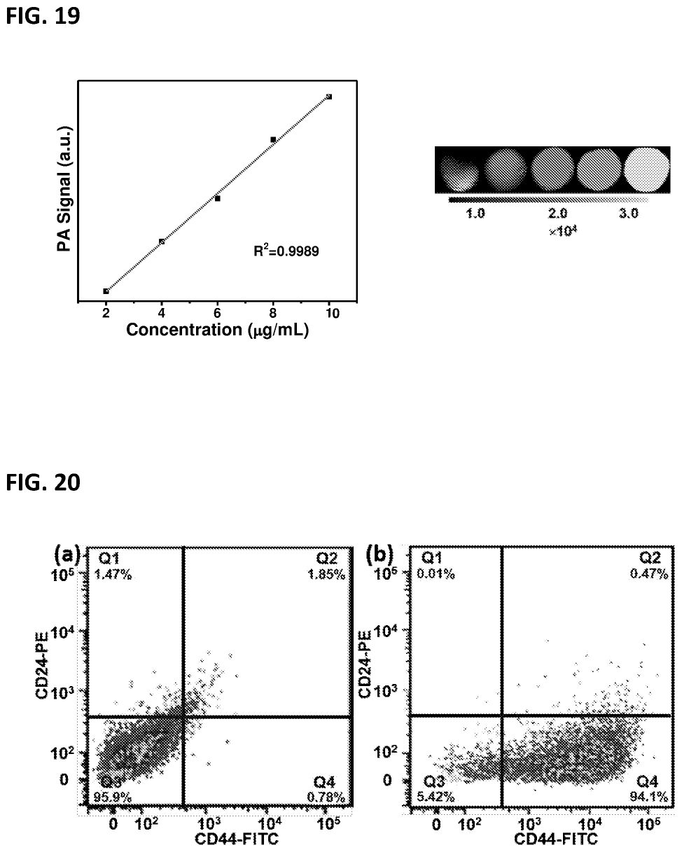

In addition to their FL properties, LAAM TC-CQDs were also found to have a capacity for photoacoustic (PA) imaging. Results in FIG. 17 and Table 2 showed that, upon radiation by a 650-nm laser (0.5 W/cm²), LAAM TC-CQDs absorbed NIR light, resulting in a thermally induced pressure jump. The resulting ultrasonic waves could be received by an acoustic detector to form PA images²⁰. The PA spectra from an agarose gel phantom filled with LAAM TC-CQDs (0-10 μg/mL) were monitored at various excitation wavelengths. The peaks at approximately 650 nm in FIG. 1e and FIG. 18 corresponded to the observed NIR absorption band of LAAM TC-CQDs shown in FIG. 1c, and the PA signal increases linearly (R²=0.9989) with LAAM TC-CQDs concentration in the range from 2 to 10 μg/mL (FIG. 19).

TABLE 2
Examples of experimentally determined photothermal conversion efficiencies (η) for
different PTCAs, listed from highest to lowest.

| | | Photothermal | |
|---|---|---|---|
| | | conversion | |
| PTT agents | Laser | efficiency (η) | Reference |
| Au bellflowers | 1 W/cm² , 808 nm | 74.0% | s3 |
| Au nanocages | 0.4 W/cm² , 808 nm | 64.0% | s4 |
| An nanomatryoshkas | Continuous-wave (CW) laser, | 63.0% | s5 |
| | 2 W/cm², 810 nm | | |
| Au/Au₂S nanoshells | 815 nm | 59.0% | s6 |
| Nanoporous Au disks | CW laser, 0.1 W/mm², | 56.0% | s7 |
| | 700-900 nm | | |
| Au nanorods | 815 nm | 55.0% | s6 |
| Au nanobipyramids | CW laser, 809 nm | 51.0% | s8 |
| Au nanorods | 2 W/cm², 808 nm | 50.0% | s9 |
| Dopamine-melanin | 2 W/cm², 808 nm | 40.0% | s10 |
| nanospheres | | | |
| Au nanoshells | CW laser, 2 W/cm², 810 nm | 39.0% | s5 |
| Red-emissive carbon dots | 2 W/cm², 671 nm | 38.5% | s11 |
| (CDs) | | | |
| Au vesicles | 1 W/cm², 808 nm | 37.0% | s12 |
| Au/SiO₂ nanoshells | 815 nm | 30.0% | s6 |
| Au hexapods | 0.4 W/cm², 808 nm | 30.0% | s4 |
| Plate-like Cu₉S₅ nanocrystals | 0.51 W/cm², 980 nm | 25.7% | s13 |
| (NCs) | | | |
| Au nanoshells | 2 W/cm², 808 nm | 25.0% | s9 |
| Au nanorods | 0.51 W/cm², 980 nm | 23.7% | s13 |
| Au nanorods | 0.4 W/cm², 808 nm | 22.0% | s4 |
| Cu₂₋ₓ SeNCs | 2 W/cm², 800 nm | 22.0% | s14 |
| Commercial Au nanorods | 2 W/cm², 800 nm | 21.0% | s14 |
| Fe₃O₄@Cu₂₋ₓS NCs | 2 W/cm², 980 nm | 16.0% | s15 |
| Commercial Au nanoshells | 2 W/cm², 800 nm | 13.0% | s14 |

NIR has the advantage of penetrating biological tissues more efficiently than visible light. Compared to photons, ultrasonic waves are less scattered; PA imaging can therefore overcome the penetration limitations of optical imaging to several centimetres²¹. With this unique NIR FL/PA dual-mode imaging capacity that combines the high contrast of optical imaging with the high spatial resolution of ultrasound, LAAM TC-CQDs represent a promising probe allowing for deep tissue imaging.

### LAAM TC-CQDs Selectively Target Cancer Cells In Vitro and In Vivo

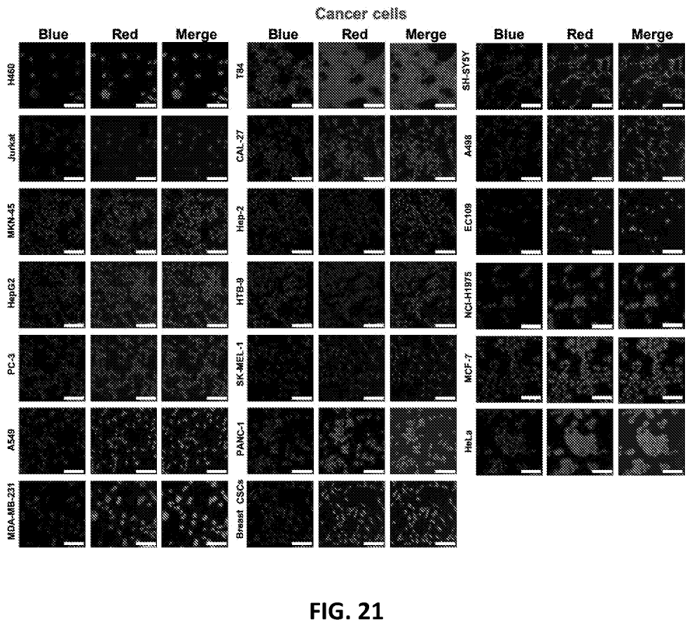

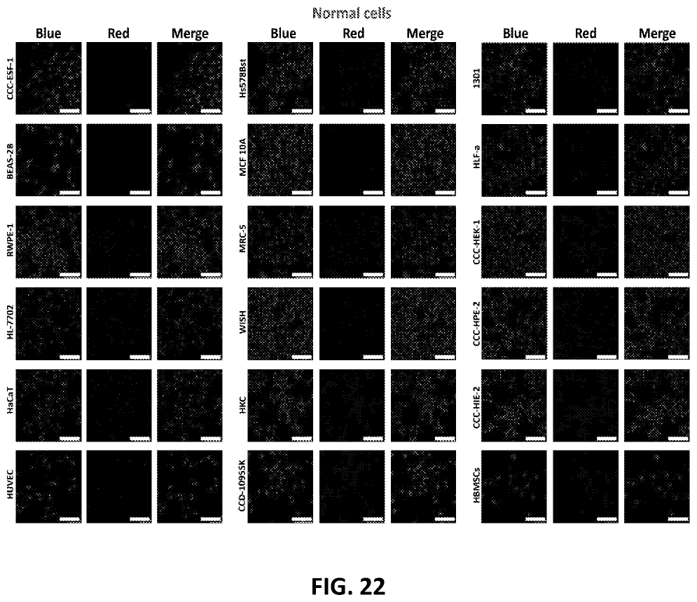

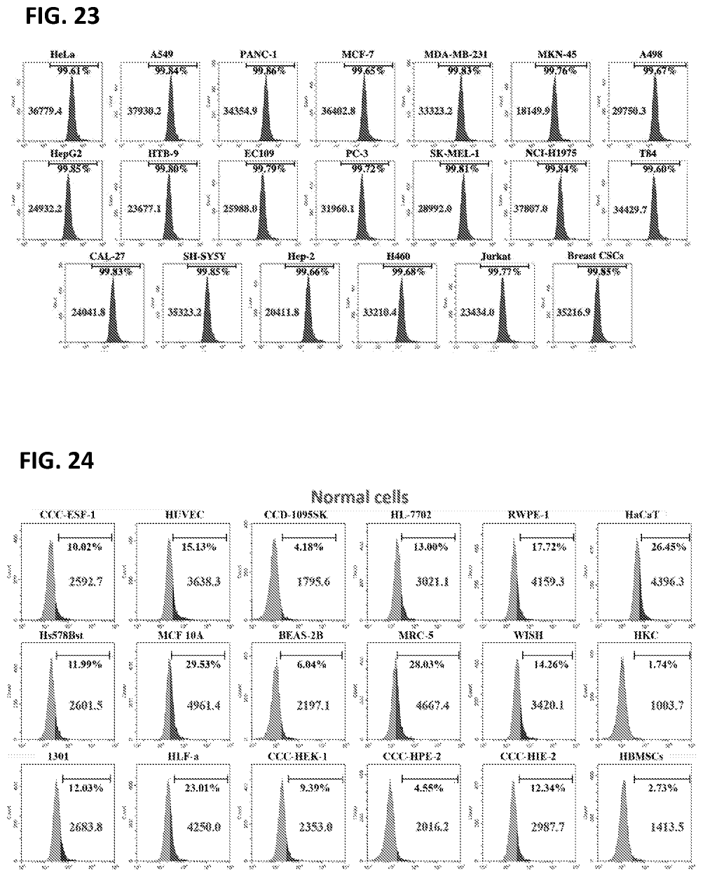

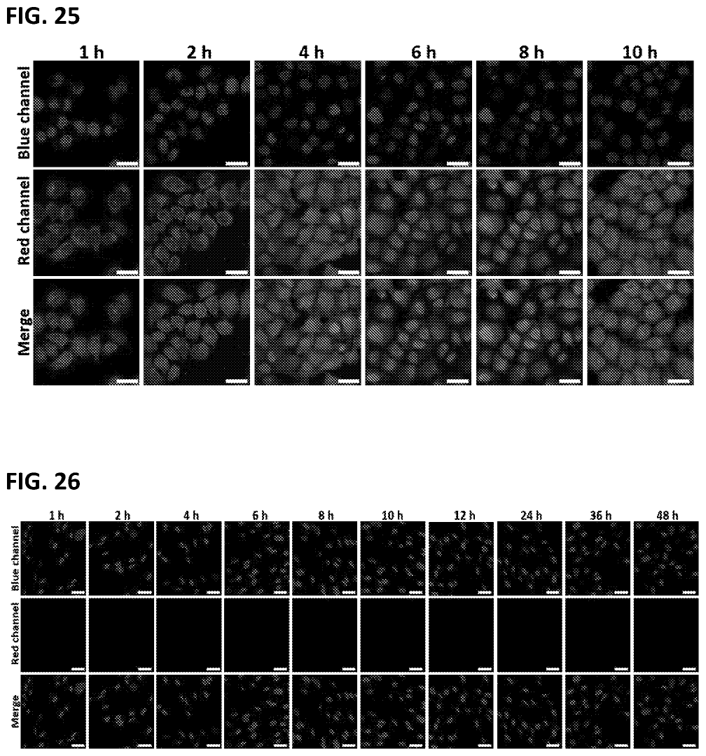

We studied the interaction between LAAM TC-CQDs and a large panel of cell cultures, including 27 cancer cell lines of different origin, a side population (SP) cancer stem-like cells isolated from MDA-MB-231 cells (FIG. 20)^{22,12} and 18 non-cancerous cell lines. A detailed list of cells used in this study is included in Table 3. Using laser confocal scanning microscopy (LCSM), we found that LAAM TC-CQDs penetrated all the tested cancer cells, regardless of their origin or stemness. In contrast, they have a limited ability to penetrate non-cancerous cells, which was confirmed by flow cytometry analysis (FIG. 2a, FIG. 21-24). Quantification analysis show that ˜99% of cancer cells (all except one BCSC line for which the rate is 95%) were positive for LAAM TC-CQDs, while the rate for non-cancerous cells was limited to <300 (1300 on average) (FIG. 2b and Table 3). Further LCSM analysis found that a significant fraction of LAAM TC-CQDs entered the nuclei, evidenced by the overlap of LAAM TC-CQDs FL with 4,6-diamidino-2-phenylindole (DAPI) FL, which stains the nuclei. To determine the kinetics of cellular penetration and nuclear localization, we incubated HeLa cells with LAAM TC-CQDs and monitored the intracellular location of LAAM TC-CQDs over time (FIG. 25). At the 1 h time point, the majority of LAAM TC-CQDs were located on the cell membrane. Subsequently, LAAM TC-CQDs penetrated into cells and localized in the cytoplasm. After 6 h, a significant portion of LAAM TC-CQDs entered the nuclei. By 8 h, the concentration of LAAM TC-CQDs in the nuclei exceeded that in the cytoplasm. We used the same procedures to monitor the interaction of LAAM TC-CQDs with non-cancerous CCC-ESF-1 cells (FIG. 26). Consistent with the observations by LCSM and flow cytometry, we did not detect interaction between LAAM TC-CQDs and representative non-cancerous CCC-ESF-1 cells throughout the entire 48 h time window.

TABLE 3
Flow cytometry analysis of the uptake of TC-CQDs in the indicated cells.

| | | Uptake | | | Uptake |
|---|---|---|---|---|---|
| Cancer cells | Origin | (%) | Normal cells | Origin | (%) |
| HeLa | Human cervical cancer | 99.6 | CCC-ESF-1 | Human embryonic skin | 10.0 |
| | cells | | | fibroblasts | |
| A549 | Human lung carcinoma | 99.8 | HUVEC | Human umbilical vein | 15.1 |
| | cells | | | endothelial cells | |
| PANC-1 | Human pancreatic | 99.9 | CCD-1095SK | Human skin fibroblasts | 4.2 |
| | carcinoma cells | | | | |
| MCF7 | Human breast | 99.7 | HL-7702 | Human hepatic cells | 13.0 |
| | adenocarcinoma cells | | | | |
| MDA-MB- | Human breast | 99.8 | RWPE-1 | Human prostate epithelial | 17.7 |
| 231 | adenocarcinoma cells | | | cells | |
| MKN-45 | Human gastric | 99.8 | HaCaT | Human keratinocyte cells | 26.5 |
| | adenocarcinoma cells | | | | |
| A498 | Human renal carcinoma | 99.7 | Hs578Bst | Human breast epithelial | 12.0 |
| | cells | | | cells | |
| HepG2 | Human hepatocellular | 99.9 | MCF 10A | Human mammary | 29.5 |
| | carcinoma cells | | | epithelial cell | |
| HTB-9 | Human bladder carcinoma | 99.8 | BEAS-2B | Human bronchial epithelial | 6.0 |
| | cells | | | cells | |
| EC109 | Human esophageal | 99.8 | MRC-5 | Human lung fibroblasts | 28.0 |
| | carcinoma cells | | | | |
| PC-3 | Human prostate | 99.7 | WISH | Human amniotic cells | 14.3 |
| | adenocarcinoma cells | | | | |
| SK-MEL-1 | Human melanoma cells | 99.8 | HKC | Human renal tubular | 1.7 |
| | | | | epithelial cells | |
| NCI-H1975 | Human lung | 99.8 | 1301 | Human T lymphocytes | 12.0 |
| | adenocarcinoma cells | | | | |
| T84 | Human colonic carcinoma | 99.6 | HLF-a | Human lung fibroblasts-a | 23.0 |
| | cells | | | | |
| CAL-27 | Human oral carcinoma | 99.8 | CCC-HEK-1 | Human embryonic kidney | 9.4 |
| | cells | | | diploid cells | |
| | | | CCC-HPE-2 | Human embryonic | 4.6 |
| | | | | pancreatic cells | |
| Hep-2 | Human laryngeal | 99.7 | CCC-HIE-2 | Human embryonic | 12.3 |
| | carcinoma cells | | | intestinal mucosa cells | |
| H460 | Human lung carcinoma | 99.7 | HBMSC | Human bone marrow | 2.7 |
| | cells | | | stromal stem cells | |
| Jurkat | Human leukemic T-cell | 99.8 | | | |
| | lymphoblast cells | | | | |
| MDA-MB- | Side population cells | 99.9 | | | |
| 231 SP cells | isolated from MDA-MB- | | | | |
| | 231 | | | | |
| 4T1 | Mouse mammary | 100.0 | | | |
| | carcinoma cells | | | | |
| MDA-MB- | Human melanoma cells | 100.0 | | | |
| 435 | | | | | |
| HCT116 | Human colorectal | 100.0 | | | |
| | carcinoma cells | | | | |
| DU145 | Human prostate cancer | 100.0 | | | |
| | cells | | | | |

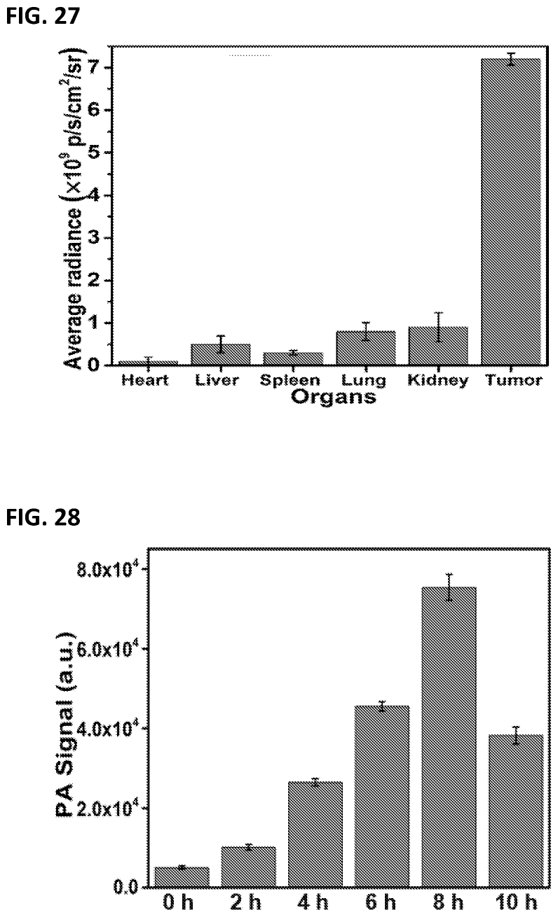

We assessed whether LAAM TC-CQDs maintain the observed high degree of tumor-specificity in vivo. LAAM TC-CQDs were injected intravenously to mice bearing HeLa tumors at 5 mg/kg. At 0, 2, 4, 6, 8 and 10 h after injection, the mice were subjected to FL/PA imaging. FIG. 2c showed that FL in the tumor region gradually increased with time and peaked at 8 h, when FL in other regions was not observed. Three-dimensional (3D) reconstruction of FL images confirmed that LAAM TC-CQDs selectively imaged tumors but not normal tissues (FIG. 2d). The high degree of selectivity was validated by ex vivo imaging of isolated organs and tumors, which showed that the FL signal in tumors was significantly greater than that of normal organs (FIG. 2e and FIG. 27). Consistent with the finding by FL imaging, the average PA intensity in tumors increased continuously with time until 8 h post-injection, when the PA signal was predominately concentrated in tumors (FIG. 2f and FIG. 28).

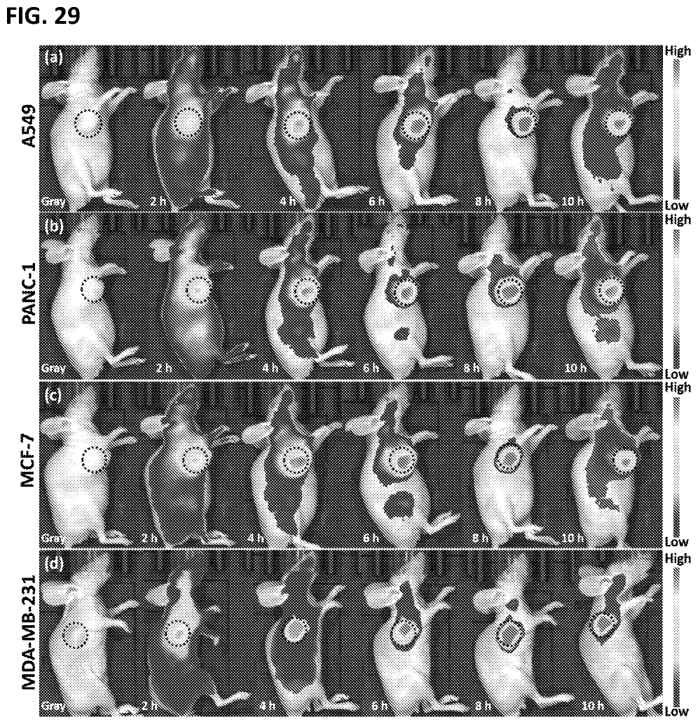

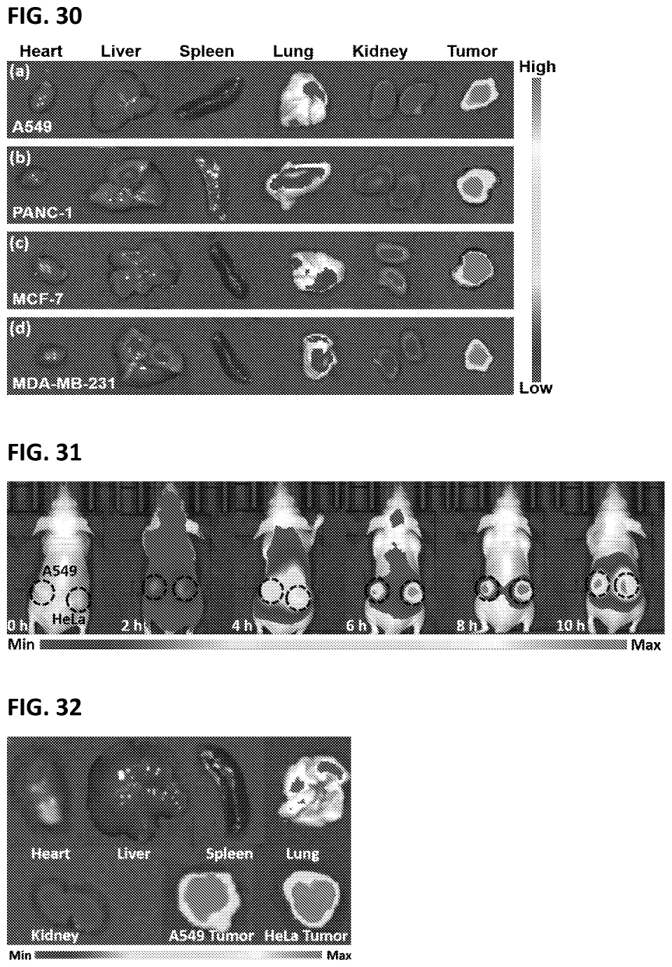

To exclude the possibility that the observed tumor-specificity is unique to HeLa tumors, we evaluated LAAM TC-CQDs in four additional tumor xenografts derived from A549 cells, PANC-1 cells, MCF-7 cells, and MD-MBA-231 cells, respectively. We found that LAAM TC-CQDs imaged tumors in all tested models, with a kinetics and specificity similar to those observed in the HeLa model (FIGS. 29 and 30). To further validate the tumor-specificity, we evaluated LAAM TC-CQDs in mice bearing a A549 lung carcinoma on the left flank and a HeLa ovarian tumor on the right flank. As shown in FIGS. 31 and 32, LAAM TC-CQDs penetrated both tumors with a comparable specificity.

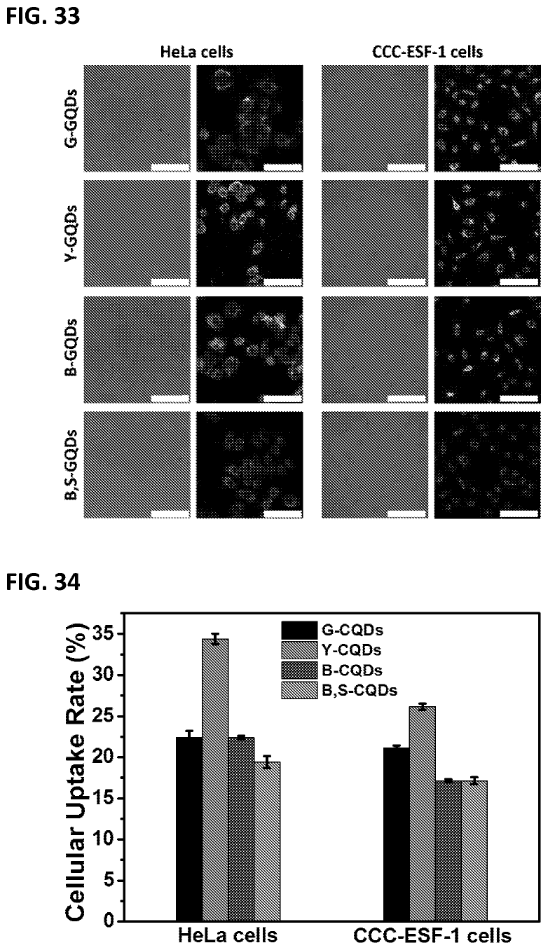

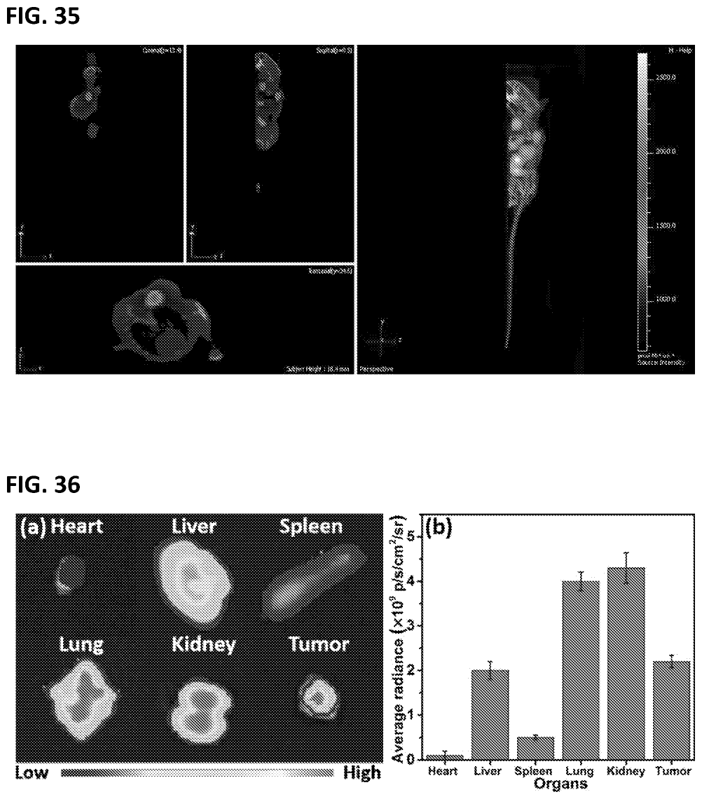

We studied whether the observed high degree of tumor-specificity could be achieved using traditional CQDs. G-CQDs¹⁷, Y-CQDs¹⁹, B-CQDs²³, and B,S-CQDs¹⁴ were synthesized as examples. These four previously reported CQDs were incubated with a panel of cancer and non-cancerous cells, and specificity was assayed using flow cytometry analysis. As shown in FIGS. 33 and 34, all the tested CQDs penetrate both cancer and non-cancerous cells without selectivity. After cell penetration, most of the CQDs were localized in the cytoplasm. G-CQDs were selected and further evaluated in mice bearing HeLa tumors. Experiments were carried out according to the same procedures that were used in the LAAM TC-CQDs study. A 3D reconstruction of FL images suggested that, in addition to the tumors, FL signal was also presented in the liver, lung and kidney (FIG. 35). Ex vivo imaging showed that the FL intensities in the lung and kidney were significantly greater than those in the tumor (FIG. 36). We evaluated whether the traditional CQDs could be engineered to reach the degree of tumor-specificity observed in LAAM TC-CQDs through conjugation of tumor-targeting ligands. We synthesized G-CQDs with surface conjugation of folic acid (FA) according to our recently published procedures¹⁸. The resulting CQDs, termed FA-G-CQDs, were evaluated in mice bearing both HeLa and A549 tumors. HeLa tumors, but not A549 tumors, are known to overexpress the FA receptor^{21,24}. As expected, we found that FA conjugation enhanced the accumulation of FA-G-CQDs in HeLa tumors, but not in A549 tumors (FIG. 37), suggesting that the ligand conjugation approach primarily enhances the targeting efficiency of CQDs for receptor-expressing tumors. Although the FL signal in HeLa tumors was greater than that of other organs, we observed significant amounts of FL signal in the liver, lung and kidney, which was confirmed by ex vivo imaging (FIG. 38).

Taken together, these data demonstrate that LAAM TC-CQDs have a unique ability to selectively interact with cancer cells, regardless of the origin, but have a limited ability to interact with non-cancerous cells. As a result of their unique NIR FL/PA dual-mode imaging capacity, LAAM TC-CQDs enable in vivo imaging of tumors with minimal background.

LAT1 Mediates the Internalization of LAAM TC-CQDs into Cancer Cells

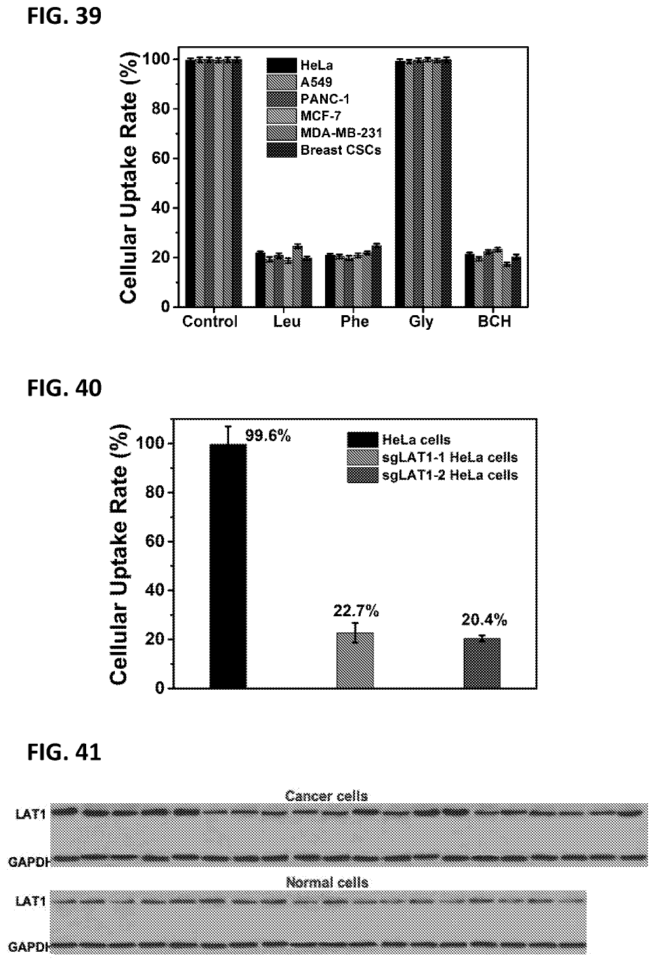

To investigate the mechanism that accounts for the cellular uptake of LAAM TC-CQDs, we treated HeLa cells with an excess of leucine (Leu), phenylalanine (Phe), or glycine (Gly) prior to adding LAAM TC-CQDs. Except Gly, both Leu and Phe are known to be high-affinity substrates to LAT1²⁷. As shown in FIG. 3a, the uptake of LAAM TC-CQDs was significantly inhibited by Leu and Phe, but not Gly. This observation leads to the hypothesis that LAAM TC-CQDs penetrate cancer cells at least in part via interaction with LAT1. This hypothetical mechanism is supported by several lines of evidence. First, pre-treatment with the LAT1 inhibitor 2-aminobicyclo-(2,2,1)-heptane-2-carboxylic acid (BCH) dramatically reduces the uptake of LAAM TC-CQDs by HeLa cells (FIG. 3a). Quantification by flow cytometry showed that pre-treatment with BCH reduced the cellular uptake rates from ˜99% to ˜20% across all tested cell lines, including HeLa, A549, PANC-1, MCF-7, MDA-MB-231, and MDA-MB-231 SP cells (FIG. 39). Second, LAT1-knockout, generated using the CRISPR/Cas9 technology and validated by sequencing and Western Blotting (WB), significantly reduced the uptake of LAAM TC-CQDs by HeLa cells by ˜80% (FIG. 3b-d and FIG. 40). Third, across various cells, the expression level of LAT1 is correlated with the amount of LAAM TC-CQDs that penetrate cells (FIG. 3e, FIG. 41 and Table 4). Consistent with previous reports, the level of LAT1 expression in cancer cells is significantly greater than that in non-cancerous cells 8,10,11

TABLE 4
The relative LAT1 expression and uptake of TC-CQDs in various types of cancer cells
and normal cells for test. (Note: the relative LAT1 expressions in different types of cells
were measured by WB tests from FIG. 37 and the uptake of TC-CQDs were obtained
from FIGS. 17 and 18).

| | Relative LAT1 | | | Relative LAT1 | |
|---|---|---|---|---|---|
| | expression | | Normal | expression | |
| Cancer cells | (LAT1/GAPDH) | Uptake | cells | (LAT1/GAPDH) | Uptake |
| HeLa | 0.88 | 36779.4 | CCC-ESF- | 0.12 | 2592.7 |
| | | | 1 | | |
| A549 | 0.89 | 37930.2 | HUVEC | 0.20 | 3638.3 |
| PANC-1 | 0.85 | 34354.9 | CCD- | 0.06 | 1795.6 |
| | | | 1095SK | | |
| MCF-7 | 0.87 | 36402.8 | HL-7702 | 0.17 | 3021.1 |
| MDA-MB- | 0.83 | 33323.2 | RWPE-1 | 0.22 | 4159.3 |
| 231 | | | | | |
| MKN-45 | 0.52 | 18149.9 | HaCaT | 0.25 | 4896.3 |
| A498 | 0.73 | 29750.3 | Hs578Bst | 0.13 | 2601.5 |
| HepG2 | 0.68 | 24932.2 | MCE 10A | 0.29 | 5561.4 |
| HTB-9 | 0.64 | 23677.1 | BEAS-2B | 0.09 | 2197.1 |
| EC109 | 0.72 | 25988 | MRC-5 | 0.26 | 5167.4 |
| PC-3 | 0.77 | 31960.1 | WISH | 0.18 | 3420.1 |
| SK-MEL-1 | 0.75 | 28992 | HKC | 0.03 | 1003.7 |
| NCI-H1975 | 0.90 | 37807.0 | 1301 | 0.14 | 2683.8 |
| T84 | 0.84 | 34429.7 | HLF-a | 0.23 | 4250 |
| CAL-27 | 0.67 | 24041.8 | CCC-HEK- | 0.11 | 2353 |
| | | | 1 | | |
| SH-SY5Y | 0.85 | 35323.2 | CCC-HPE- | 0.08 | 2016.2 |
| | | | 2 | | |
| Hep-2 | 0.61 | 20411.8 | CCC-HIE- | 0.16 | 2987.7 |
| | | | 2 | | |
| H460 | 0.79 | 33210.4 | HBMSCs | 0.05 | 1413.5 |
| Jurkat | 0.68 | 23434 | | | |
| Breast CSCs | 0.86 | 35216.9 | | | |

Taken together, our results suggest that the interaction between LAAM TC-CQDs and cancer cells is mediated by LAT1.

### LAAM TC-CQDs for Tumor-Specific Drug Delivery

Major limitations associated with traditional chemotherapy include significant adverse toxicity to normal tissues and intrinsic or acquired drug resistance as a result of overexpression of multidrug resistance ATP binding cassette (ABC) transporters. The efficacy of DNA-damaging chemotherapy drugs, which function through interaction with DNA within the nuclei, are often further reduced by their limited ability to penetrate the nucleus²⁸. Those limitations can be potentially overcome by using LAAM TC-CQDs as a drug carrier. First, LAAM TC-CQDs have tremendous tumor-specific targeting, making it possible to avoid systemic toxicity. Second, due to their nano size, LAAM TC-CQDs are not subjected to removal by ABC transporters. Third, LAAM TC-CQDs efficiently penetrate the nucleus, making it possible to maximize the efficacy of DNA damaging chemotherapy.

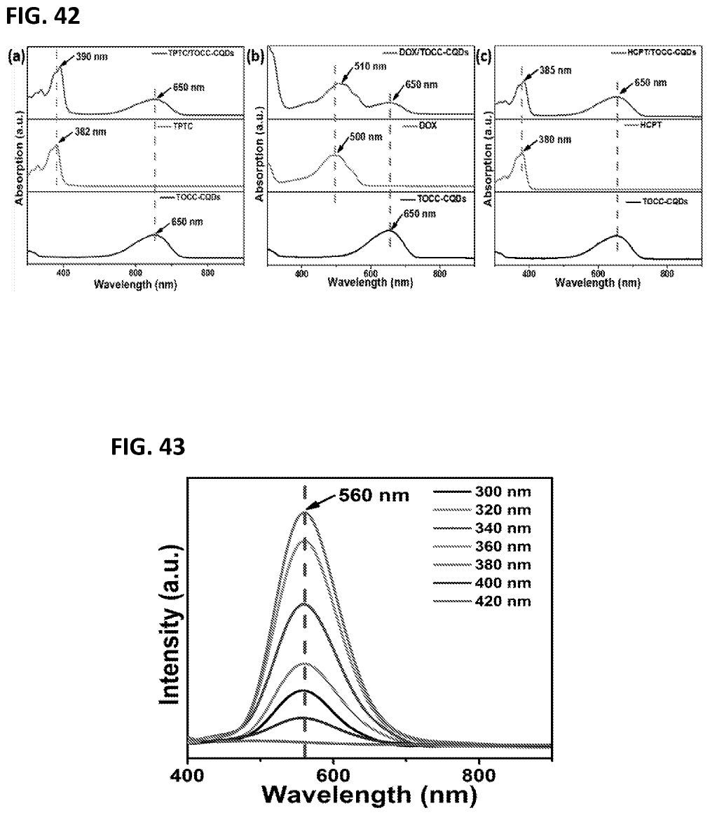

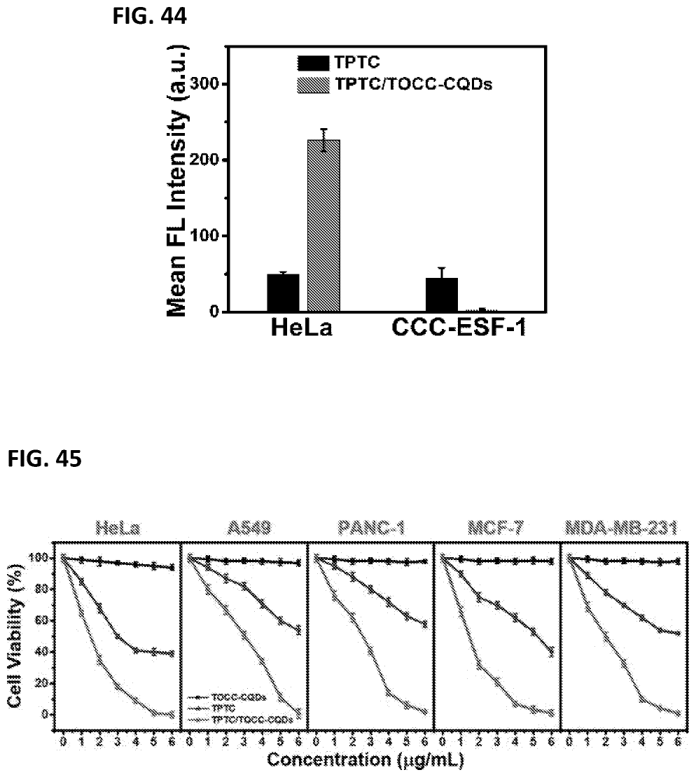

We evaluated LAAM TC-CQDs as a drug carrier for the delivery of DNA damaging chemotherapy drugs. LAAM TC-CQDs bearing the large π-conjugated structure, which allows loading aromatic chemotherapy drugs, such as topotecan hydrochloride (TPTC), doxorubicin (DOX), and hydroxycamptothecin (HCPT), through a π-π stacking interaction (FIG. 42). We further evaluated TPTC-loaded LAAM TC-CQDs, designated as TPTC/TC-CQDs, which were synthesized by mixing LAAM TC-CQDs with TPTC overnight followed with extensive dialysis. Successful loading of TPTC was evidenced by the characteristic UV-vis absorbance peak at 390 nm that is superimposed on the absorption spectrum of LAAM TC-CQDs (FIG. 42a). The slight redshift of the absorption peak of TPTC upon loading could be attributed to the 71-71 stacking interaction between TPTC and LAAM TC-CQDs. TPTC/TC-CQDs bear dual FL emissions corresponding to TPTC (yellow channel, FIG. 43) and LAAM TC-CQDs (red channel, FIG. 1d). We characterized the interaction between TPTC/TC-CQDs and a panel of cancer cells and non-cancerous cells. As shown in FIG. 4a, TPTC/TC-CQDs maintain the selective penetrability to cancer cells, and delivery via LAAM TC-CQDs eliminates the penetration of TPTC to non-cancerous cells. In particular, based on FL intensity, delivery via LAAM TC-CQDs enhanced the accumulation of TPTC in the nuclei by 4.5-fold (FIG. 44). Delivery via LAAM TC-CQDs significantly enhanced the toxicity of TPTC to cancer cells but reduced its toxicity to non-cancerous cells. At a concentration equivalent to 5 ug/mL TPTC, TPTC/TC-CQDs killed all HeLa cells but did not exhibit toxicity to non-cancerous CCC-ESF-1 cells (FIG. 4b). By contrast, free TPTC at the same concentration inhibited both cells at a comparable efficiency. The significant toxicity enhancing effect to cancer cells is not unique to HeLa cells, as a comparable degree of enhancement was also seen in other cancer cells, including A549, PANC-1, MCF-7 and MDA-MB-231 cells (FIG. 45). This clearly demonstrates that the LAAM CPDs of the invention can be used to carry chemotherapeutic cargo selectively into cancerous cells, greatly enhancing the efficacy and safety of cytotoxic therapeutic agents.

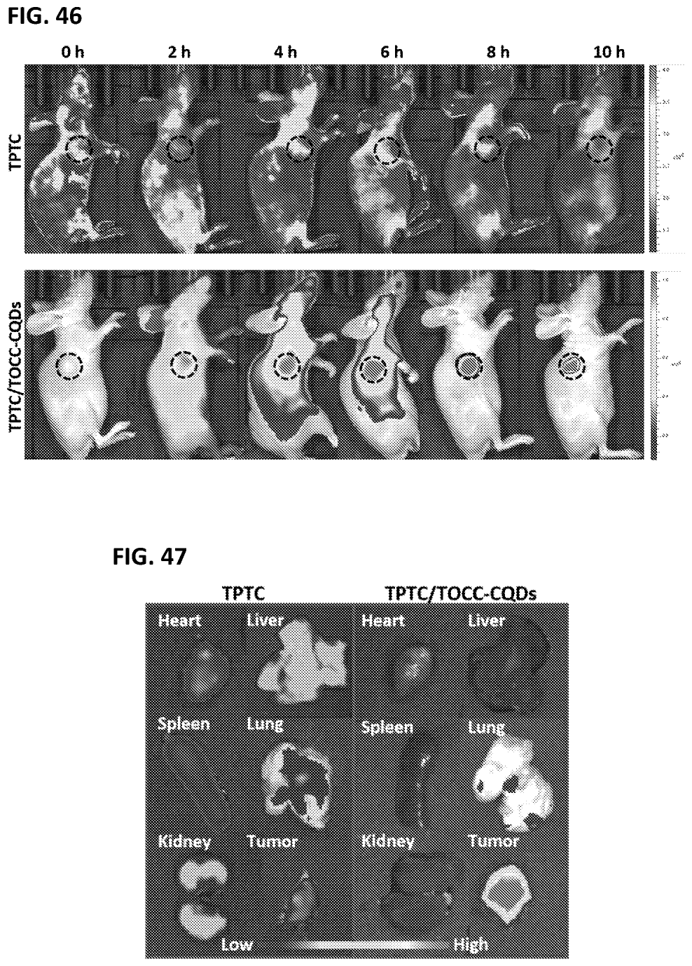

We determined the pharmacokinetics of TPTC/TC-CQDs in mice bearing HeLa tumors. TPTC or TPTC/TC-CQDs were administered intravenously at a dose equivalent to 10 mg/kg TPTC, and the blood was collected at various time points. TPTC in the plasma was quantified by high performance liquid chromatography (HPLC). As shown in FIG. 4c, delivery via LAAM TC-CQDs significantly enhanced the blood circulation and bioavailability of TPTC, with the half-life of TPTC increasing from 5 min to 40 min and the area under the curve (AUC) increasing from 13.4 to 138.1. A second cohort of mice received the same treatment was subjected to characterization for biodistribution of TPTC based on TPTC fluorescence. As expected, free TPTC was widely distributed over the body without specificity. By contrast, TPTC/TC-CQDs exhibited a kinetics similar to that of LAAM TC-CQDs (FIG. 46). By 8 h, TPTC could only be detected in tumors. Ex vivo imaging of isolated organs confirmed that LAAM TC-CQDs enabled delivery of TPTC to tumors with high specificity (FIG. 47). Quantification based on TPTC FL intensity showed that delivery via LAAM TC-CQDs increased the concentration of TPTC in tumors by 13.8-fold while significantly reducing the accumulation of TPTC in other organs (FIG. 4d).

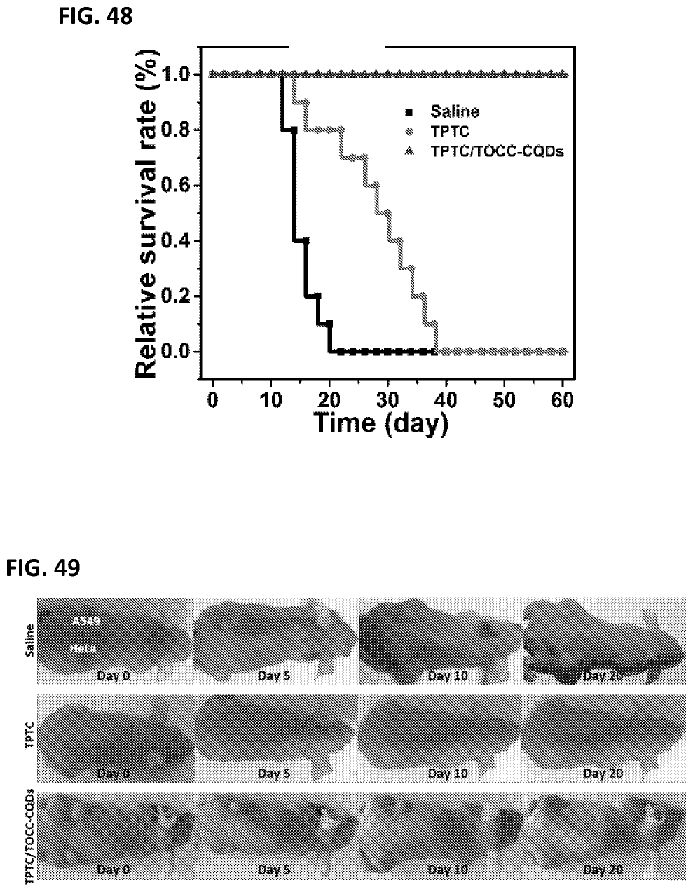

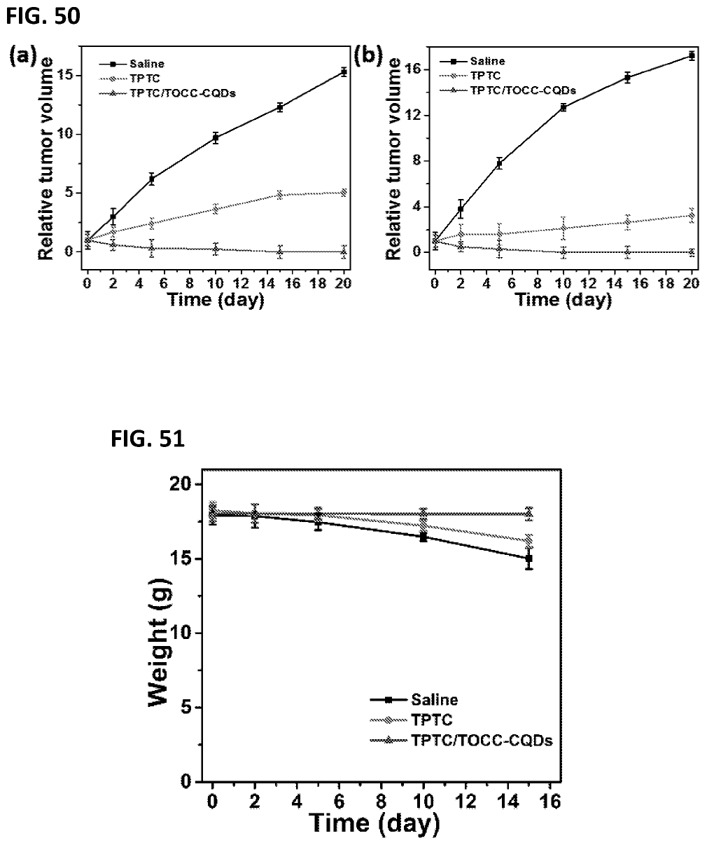

We assessed TPTC/TC-CQDs for cancer treatment using mice bearing HeLa tumors. When tumor volumes reached ˜100 mm3, the mice were randomly grouped and received intravenous administration of either saline, TPTC (10 mg/kg), or TPTC/TC-CQDs (equivalent to 10 mg/kg TPTC) at days 1, 4, 8, and 12. Fifteen days later, tumors in the mice receiving TPTC/TC-CQDs treatment shrank to an undetectable level; by contrast, tumors in the mice receiving free TPTC continuously grew, although in a rate that was slower than that of control mice (FIG. 4e-g). All the mice treated with TPTC/TC-CQDs survived over 60 days; in contrast, the mice treated with TPTC and saline survived by 40 and 20, days (FIG. 48). The significant anti-tumor effect of TPTC/TC-CQDs is not unique to HeLa tumors; we applied the same treatment to mice bearing an A549 tumor on the left flank and a HeLa tumor on the right flank. Twenty days after treatment, tumors in both sides were eradicated (FIGS. 49 and 50).

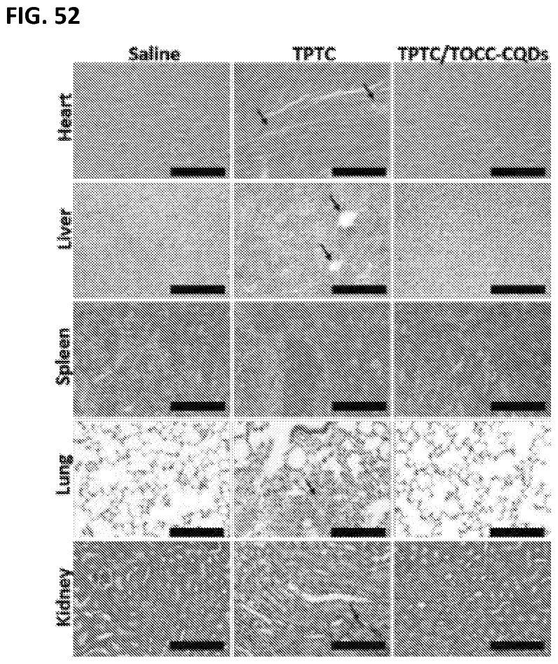

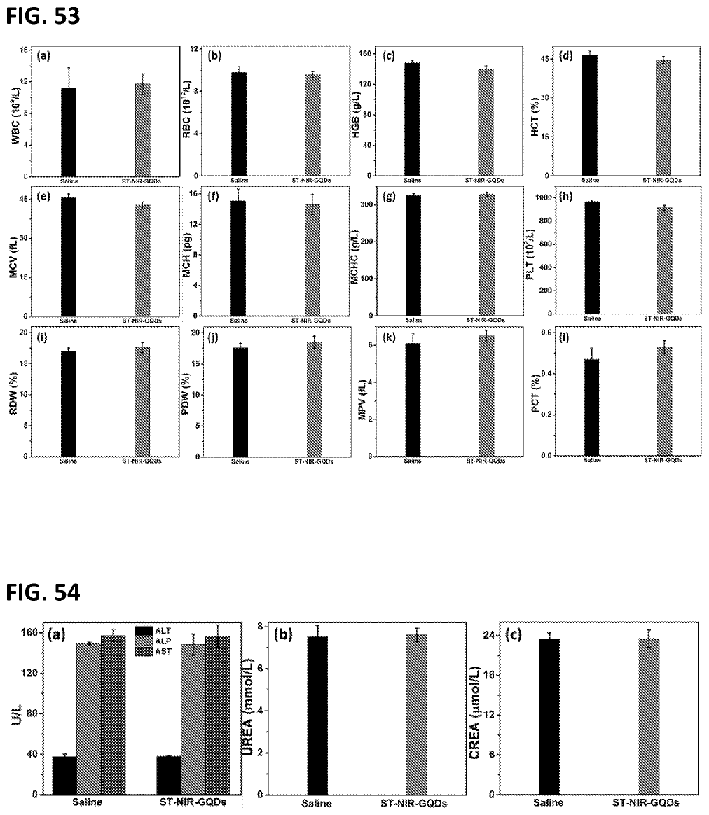

TPTC/TC-CQDs exhibited minimal toxicity to mice. As shown in FIG. 51, treatment with TPTC/TC-CQDs did not induce obvious weight loss throughout the study. By contrast, weight loss was observed in the group receiving free TPTC. Consistent with this finding, H&E analysis of major organs isolated from mice receiving TPTC/TC-CQDs treatment did not detect inflammatory infiltration or pathological damage (FIG. 52). However, obvious myocardial fiber rupture, diffuse ballooning degeneration of hepatocytes, pulmonary hyperemia, and focal tubular necrosis were found in mice treated with free TPTC. A complete blood count analysis showed that, comparable to saline control mice, standard parameters in mice receiving TPTC/TC-CQDs treatment, including white blood cells (WBC), red blood cells (RBC), hemoglobin (HGB), hematocrit (HCT), mean corpuscular volume (MCV), mean corpuscular hemoglobin (MCH), mean corpuscular hemoglobin concentration (MCHC), platelets (PLT), red cell distribution width (RDW), platelet distribution width (PDW), platelet volume (MPV) and thrombocytocrit (PCT), fell within the normal ranges (FIG. 53), suggesting that TPTC/TC-CQDs are hemocompatible. Serum biochemistry analyses, including alanine aminotransferase (ALT), aspartate aminotransferase (AST), and alkaline phosphatase (ALP) tests for hepatic function, and urea nitrogen (UREA) and creatinine (CREA) for renal function, did not detect abnormality in TPTC/TC-CQDs-treated mice (FIG. 54).

Collectively, these data show that LAAM CQDs can be employed as a safe carrier for tumor-specific delivery of chemotherapy, and delivery via LAAM CQDs significantly enhances the therapeutic benefit while reducing the systemic toxicity of cargo therapeutic agents.

### LAAM CQDs as a Novel Class of CQDs for Tumor-Specific Imaging and Drug Delivery

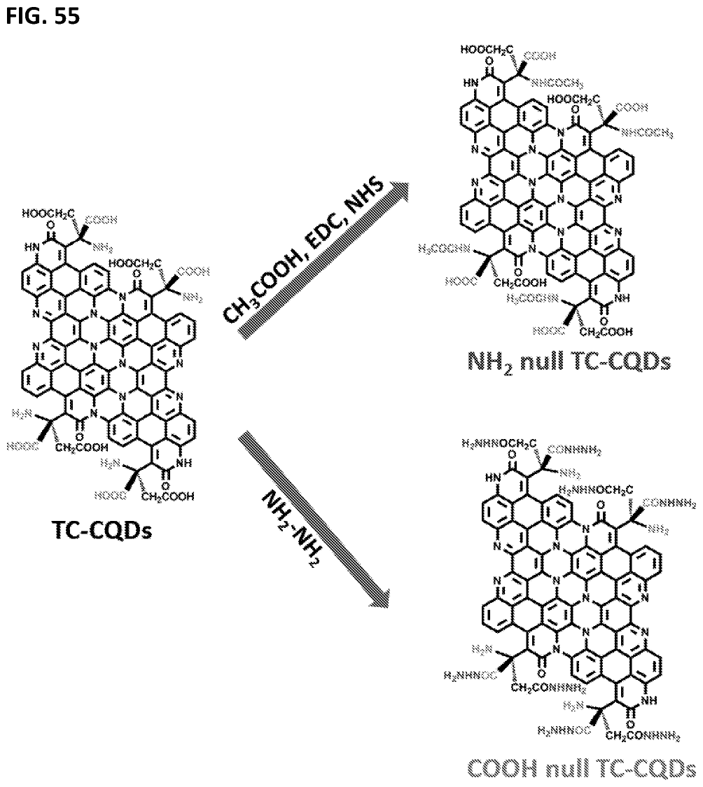

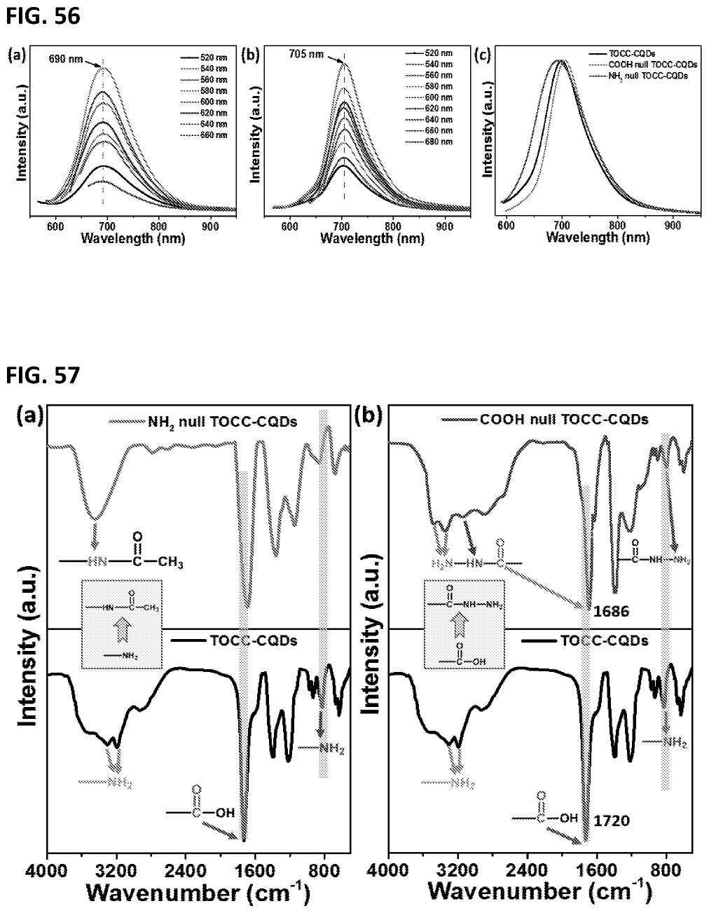

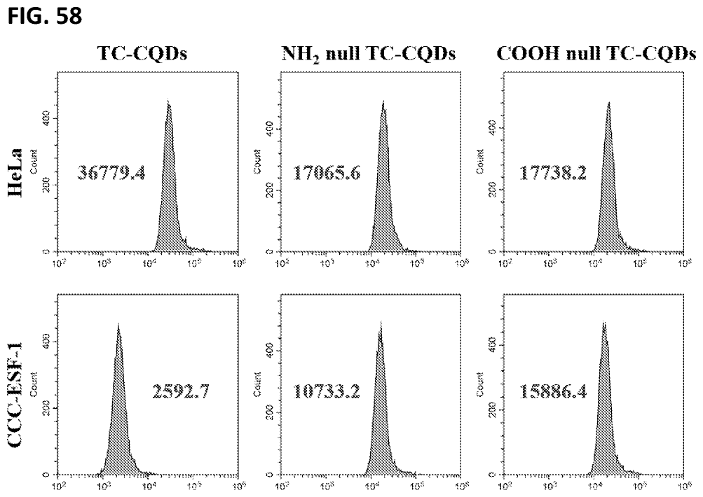

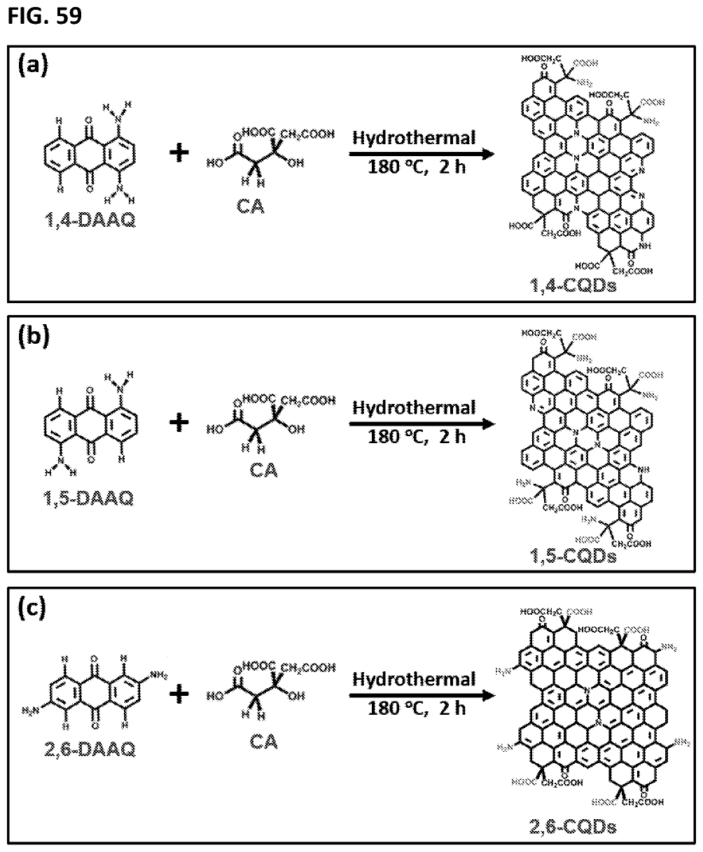

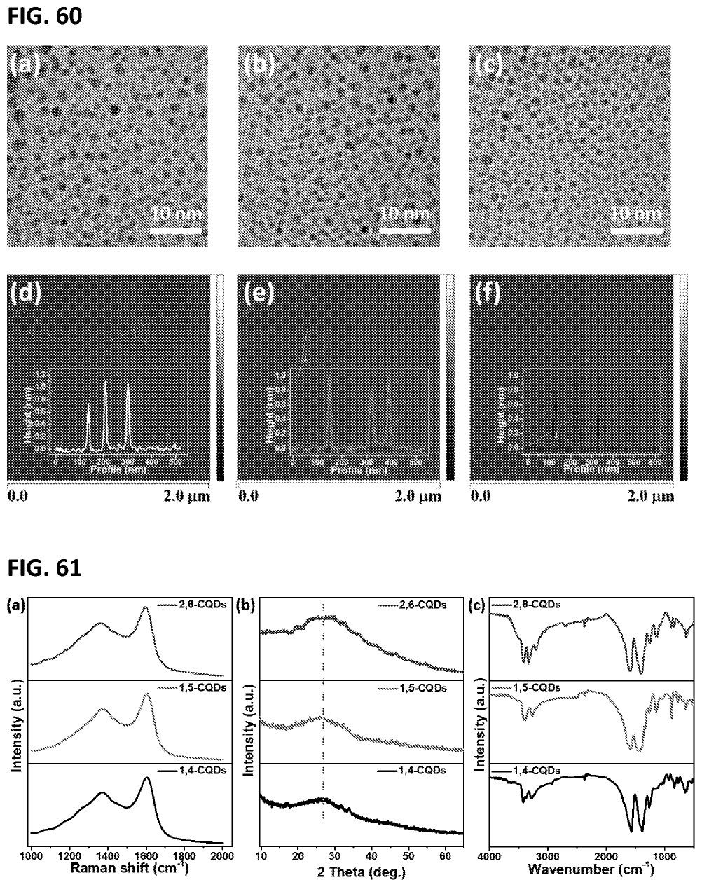

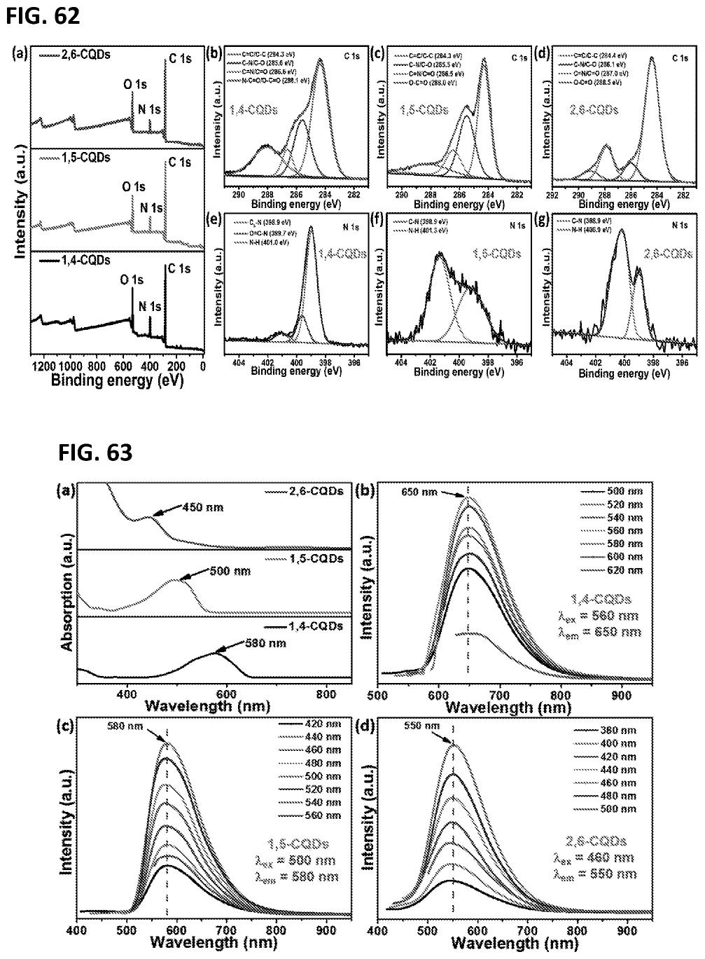

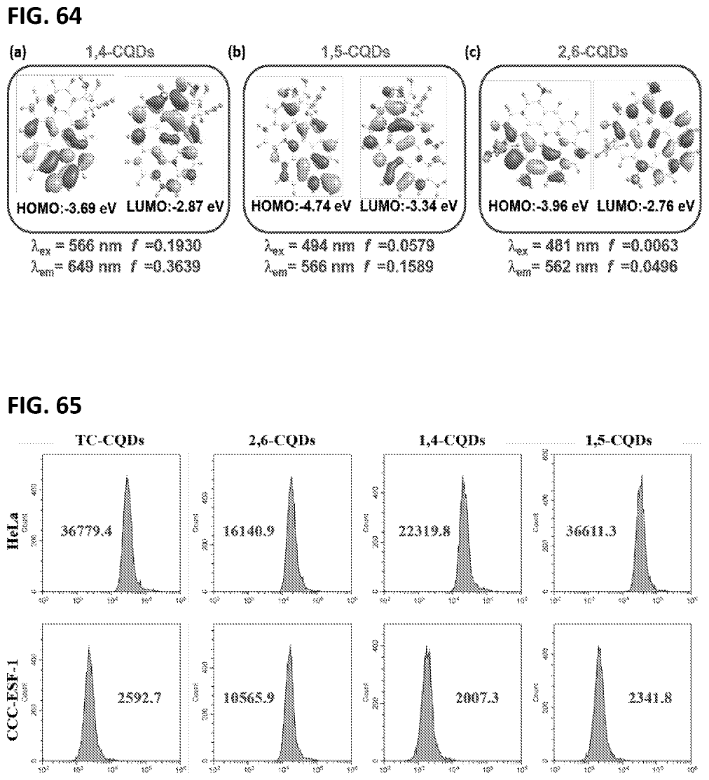

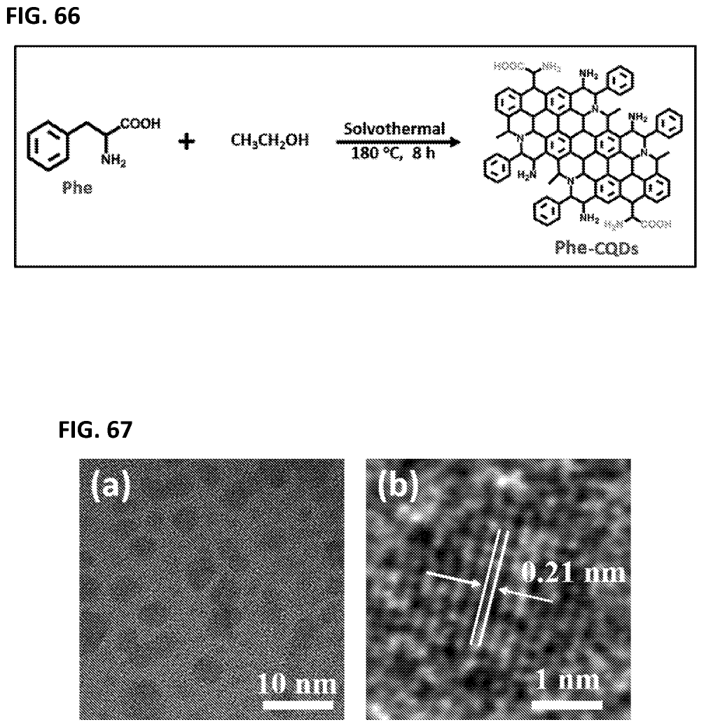

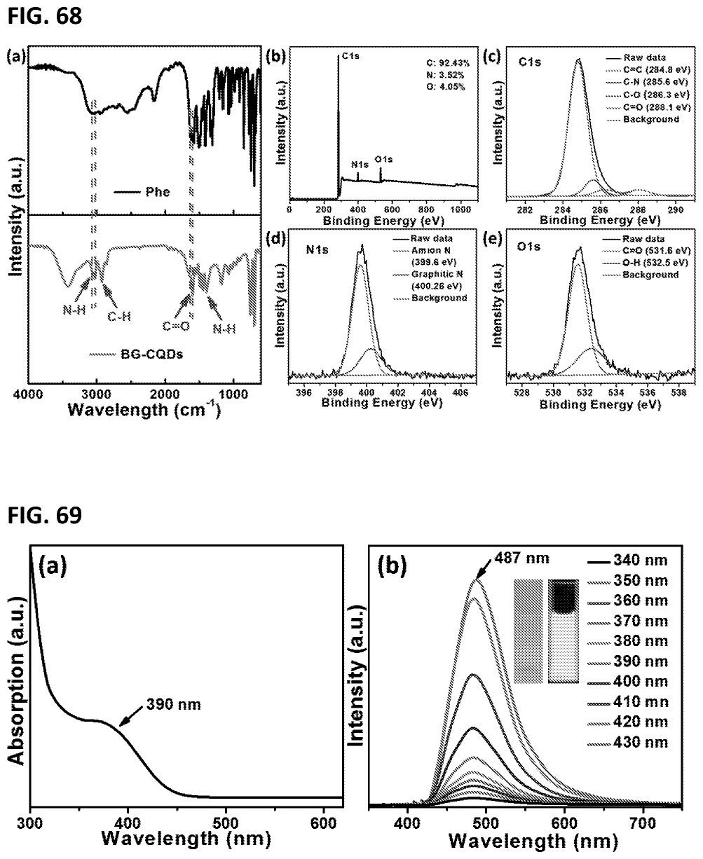

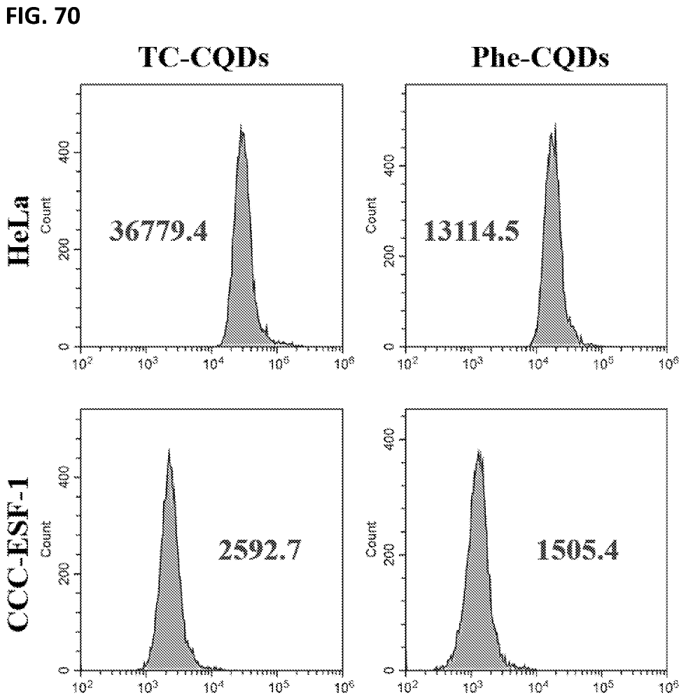

To determine the molecular structure that accounts for the tumor-specificity observed in LAAM TC-CQDs, we systematically analyzed an array of TC-CQD analogs. First, we determined whether the carboxyl groups, the amino groups, or both are essential. We removed the carboxyl and amino groups on the edge of LAAM TC-CQDs through hydrazine hydrate (NH₂—NH₂) reduction¹⁹, and carbodiimide (EDC)/N-hydroxysuccinimide (NHS) coupling reaction with acetic acid¹⁸, respectively (FIG. 55-56). The resulting NH₂— and COOH-null LAAM TC-CQDs were evaluated in HeLa and CCC-ESF-1 cells. As shown in FIG. 5a,b, the removal of either carboxyl or amino groups abolished the tumor-specific-penetrability of LAAM TC-CQDs, and the resulting CQDs penetrated both cancer and non-cancerous cells without selectivity. Flow cytometry analysis (FIG. 58) revealed that, compared to unmodified LAAM TC-CQDs, LAAM TC-CQDs without NH₂ groups and COOH groups had 46% and 48% reduced uptake in HeLa, respectively, and 414% and 613% increased uptake non-cancerous CCC-ESF-1 cells (FIG. 5c). Next, we tested whether the presence of a carboxyl group and an amino group on the same α position is essential. 2,6-CQDs, which bear the same number of carboxyl and amino groups, were synthesized using 2,6-diaminoanthraquinone (2,6-DAAQ) and CA and characterized for its structure and optical properties (FIG. 59-64 and Table 5). All of the carboxyl and amino groups of 2,6-CQDs are located in different positions relative to LAAM TC-CQDs. LCSM and flow cytometry analysis showed that the altered carboxyl and amino positions renders a complete loss of tumor-specific-penetrability; 2,6-CQDs penetrate both HeLa and CCC-ESF-1 cells with comparable efficiency (FIG. 5a-c and FIG. 65). We assessed whether the number of paired α-carboxyl and amino groups associates with tumor-specific penetrability. 1,4-CQDs and 1,5-CQDs were synthesized under the same conditions as LAAM TC-CQDs while TAAQ was replaced with 1,4-diaminoanthraquinone (1,4-DAAQ) or 1,5-diaminoanthraquinone (1,5-DAAQ). Both CQDs were characterized for its structure and optical properties (FIG. 59-64 and Table 5). 1,4-CQDs, which are structurally similar to LAAM TC-CQDs but bear half as many paired groups, maintain the tumor-specific penetrability (FIG. 5a). However, they exhibited significantly reduced penetration efficiency, which is 61% of that for LAAM TC-CQDs (FIG. 5b,c). 1,5-CQDs, which are also structurally similar to LAAM TC-CQDs and bear the same number of paired groups, penetrated HeLa cells with efficiency comparable to LAAM TC-CQDs (FIG. 5a-c). Finally, we determined whether the backbone structure of LAAM TC-CQDs is important. Phe-CQDs, which bear paired α-carboxyl and amino groups on α positions but have a significantly different backbone from LAAM TC-CQDs, were synthesized using Phe and characterized for its structure and optical properties (FIG. 65-69). Evaluation in HeLa and CCC-ESF-1 cells found that Phe-CQDs maintain the tumor-specific penetrability, although the uptake of Phe-CQDs in HeLa cells is 36% of that of LAAM TC-CQDs (FIG. 5a-c and FIG. 70). The lower penetrability of Phe-CQDs might be attributed to the poor water solubility of Phe-CQDs, which could not be dispersed in solution well and thus have reduced cellular uptake.

TABLE 5
Optimized cartesian coordinates (Å) of one FL unit of 1,4-CQDs,
1,5-CQDs and 2,6-CQDs for ground state and excited state.

| | Species | Cartesian coordinates | | | |
|---|---|---|---|---|---|
| 1,4- | Ground state | C | 2.644417000 | 0.793691000 | −0.266490000 |
| CQDs | | C | 3.404016000 | 2.002334000 | −0.589448000 |
| | | C | 2.669177000 | 3.278897000 | −0.945151000 |
| | | C | 1.226567000 | 3.313785000 | −0.559133000 |
| | | C | 0.548572000 | 2.111293000 | −0.425828000 |
| | | C | 1.264486000 | 0.820505000 | −0.487228000 |
| | | C | 0.424828000 | −0.354435000 | −0.755181000 |
| | | C | 0.931391000 | −1.483481000 | −1.420222000 |
| | | N | 4.370709000 | 0.234977000 | 1.455209000 |
| | | C | 3.524361000 | −0.319044000 | 0.369268000 |
| | | C | −0.852082000 | 2.124029000 | −0.108199000 |
| | | C | −1.586270000 | 0.928961000 | −0.048403000 |
| | | C | −0.961864000 | −0.316272000 | −0.435332000 |
| | | C | 0.554017000 | 4.548593000 | −0.354006000 |
| | | C | −0.766501000 | 4.582854000 | 0.011612000 |
| | | C | −1.517624000 | 3.375614000 | 0.153190000 |
| | | C | −1.758689000 | −1.466728000 | −0.596833000 |
| | | C | −1.213278000 | −2.625091000 | −1.207924000 |
| | | C | 0.116403000 | −2.588810000 | −1.644181000 |
| | | C | −2.861622000 | 3.376147000 | 0.534872000 |
| | | C | −3.607470000 | 2.183539000 | 0.619618000 |
| | | C | −2.960048000 | 0.943171000 | 0.278664000 |
| | | C | −3.708372000 | −0.267804000 | 0.265770000 |
| | | N | −3.088483000 | −1.459624000 | −0.186037000 |
| | | O | 4.645414000 | 1.997396000 | −0.647636000 |
| | | C | −4.973759000 | 2.146771000 | 1.001426000 |
| | | C | −5.656586000 | 0.950654000 | 1.035308000 |
| | | C | −5.038113000 | −0.264719000 | 0.668627000 |
| | | N | −5.762734000 | −1.483423000 | 0.734415000 |
| | | C | −5.220528000 | −2.571167000 | 0.046221000 |
| | | C | −3.939222000 | −2.605504000 | −0.380871000 |
| | | C | −3.358363000 | −3.756130000 | −1.020160000 |
| | | C | −2.066999000 | −3.780065000 | −1.414355000 |
| | | C | 4.506903000 | −0.908643000 | −0.689630000 |
| | | O | 4.161292000 | −1.252972000 | −1.803694000 |
| | | O | 5.767203000 | −1.023794000 | −0.277085000 |
| | | C | 2.720956000 | −1.469644000 | 1.041146000 |
| | | C | 3.602016000 | −2.389282000 | 1.877260000 |
| | | O | 4.477018000 | −3.082092000 | 1.404282000 |
| | | O | 3.350675000 | −2.453788000 | 3.202747000 |
| | | H | 2.757718000 | 3.389954000 | −2.037710000 |
| | | H | 3.228946000 | 4.118746000 | −0.522341000 |
| | | H | 1.954530000 | −1.478481000 | −1.778865000 |
| | | H | 4.747615000 | 1.135697000 | 1.171757000 |
| | | H | 3.836426000 | 0.370506000 | 2.308799000 |
| | | H | 1.112172000 | 5.473117000 | −0.470341000 |
| | | H | −1.262214000 | 5.531560000 | 0.196151000 |
| | | H | 0.513074000 | −3.453037000 | −2.168839000 |
| | | H | −3.348323000 | 4.319640000 | 0.766105000 |
| | | H | −5.476291000 | 3.071317000 | 1.266897000 |
| | | H | −6.699508000 | 0.925813000 | 1.338758000 |
| | | H | −6.767802000 | −1.387404000 | 0.666267000 |
| | | H | −5.868855000 | −3.425385000 | −0.096469000 |
| | | H | −4.007188000 | −4.613130000 | −1.168917000 |
| | | H | −1.646817000 | −4.655171000 | −1.898634000 |
| | | H | 5.754520000 | −0.715170000 | 0.663502000 |
| | | H | 2.250944000 | −2.105870000 | 0.295034000 |
| | | H | 1.917412000 | −1.027570000 | 1.637207000 |
| | | H | 2.610058000 | −1.880762000 | 3.451179000 |
| | Excited state | C | −0.394560000 | 4.502287000 | 0.057048000 |
| | | C | −1.265363000 | 3.367964000 | 0.061342000 |
| | | C | −1.759088000 | −1.523931000 | −0.147711000 |
| | | C | −1.180863000 | −2.831461000 | −0.252301000 |
| | | C | 0.216846000 | −2.954287000 | −0.331042000 |
| | | C | −2.656460000 | 3.486646000 | 0.131329000 |
| | | C | −3.511001000 | 2.353628000 | 0.124978000 |
| | | C | −2.916291000 | 1.048807000 | 0.036880000 |
| | | C | −3.736584000 | −0.106620000 | 0.019869000 |
| | | N | −3.145026000 | −1.384009000 | −0.073852000 |
| | | O | 4.872498000 | 1.436376000 | −0.517417000 |
| | | C | −4.919928000 | 2.445800000 | 0.198944000 |
| | | C | −5.707629000 | 1.302060000 | 0.185010000 |
| | | C | −5.135026000 | 0.030032000 | 0.095701000 |
| | | N | −5.931510000 | −1.130418000 | 0.078749000 |
| | | C | −5.364992000 | −2.356234000 | −0.016644000 |
| | | C | −4.000302000 | −2.531445000 | −0.094727000 |
| | | C | −3.402843000 | −3.808385000 | −0.196884000 |
| | | C | −2.045142000 | −3.968128000 | −0.274656000 |
| | | C | 4.248465000 | −1.085539000 | −1.627821000 |
| | | O | 5.196956000 | −1.835257000 | −1.699139000 |
| | | O | 3.780520000 | −0.466068000 | −2.719654000 |
| | | C | 4.364359000 | −1.164336000 | 0.874090000 |
| | | C | 3.713791000 | −1.183342000 | 2.242045000 |
| | | O | 2.545290000 | −0.979121000 | 2.484651000 |
| | | O | 4.561618000 | −1.452766000 | 3.264759000 |
| | | H | 3.518540000 | 3.326102000 | 0.852887000 |
| | | H | 3.515191000 | 3.625144000 | −0.862058000 |
| | | H | 2.782827000 | −2.922883000 | −0.401525000 |
| | | H | 1.610582000 | 5.225794000 | −0.008933000 |
| | | H | −0.830461000 | 5.495831000 | 0.108082000 |
| | | H | 0.646955000 | −3.947957000 | −0.411890000 |
| | | H | −3.102912000 | 4.475393000 | 0.190271000 |
| | | H | −5.386662000 | 3.422981000 | 0.267156000 |
| | | H | −6.789163000 | 1.384996000 | 0.243173000 |
| | | H | −6.935084000 | −1.040914000 | 0.129505000 |
| | | H | −6.026788000 | −3.211296000 | −0.030067000 |
| | | H | −4.065699000 | −4.666875000 | −0.212541000 |
| | | H | −1.609615000 | −4.958079000 | −0.353900000 |
| | | H | 3.067272000 | 0.144414000 | −2.454085000 |
| | | H | 5.129309000 | −0.381410000 | 0.850394000 |
| | | H | 4.884141000 | −2.114365000 | 0.707002000 |
| | | H | 5.466571000 | −1.587511000 | 2.942407000 |
| 1,5- | Ground state | C | 2.853663000 | 0.673595000 | −0.281693000 |
| CQDs | | C | 3.679242000 | 1.834650000 | −0.612134000 |
| | | C | 3.025108000 | 3.168637000 | −0.917546000 |
| | | C | 1.595546000 | 3.289923000 | −0.505127000 |
| | | C | 0.847407000 | 2.122578000 | −0.386273000 |
| | | C | 1.470017000 | 0.784652000 | −0.473158000 |
| | | C | 0.545499000 | −0.328737000 | −0.727121000 |
| | | C | 0.963873000 | −1.530462000 | −1.372487000 |
| | | N | 4.590110000 | 0.005265000 | 1.393456000 |
| | | C | 3.673806000 | −0.492834000 | 0.338225000 |
| | | C | −0.540396000 | 2.230148000 | −0.073288000 |
| | | C | −1.358066000 | 1.090231000 | −0.039540000 |
| | | C | −0.828223000 | −0.188044000 | −0.436395000 |
| | | C | 0.998241000 | 4.553135000 | −0.275241000 |
| | | C | −0.325367000 | 4.665451000 | 0.086214000 |
| | | C | −1.140075000 | 3.505370000 | 0.197496000 |
| | | C | −1.727748000 | −1.285422000 | −0.594366000 |
| | | C | −1.254311000 | −2.516285000 | −1.131188000 |
| | | C | 0.099989000 | −2.583132000 | −1.563424000 |
| | | N | −2.441412000 | 3.647194000 | 0.550079000 |
| | | C | −3.211376000 | 2.552560000 | 0.590430000 |
| | | C | −2.719040000 | 1.242329000 | 0.266836000 |
| | | C | −3.586004000 | 0.128223000 | 0.221752000 |
| | | C | −3.103746000 | −1.153222000 | −0.226322000 |
| | | O | 4.915768000 | 1.750817000 | −0.710383000 |
| | | C | −4.601554000 | 2.668009000 | 0.936739000 |
| | | C | −5.430794000 | 1.580054000 | 0.918884000 |
| | | C | −4.936935000 | 0.288403000 | 0.540810000 |
| | | N | −5.763936000 | −0.791128000 | 0.472004000 |
| | | C | −5.392135000 | −2.095739000 | 0.083521000 |
| | | C | −3.980075000 | −2.245609000 | −0.332933000 |
| | | C | −3.478714000 | −3.464453000 | −0.852707000 |
| | | C | −2.165117000 | −3.595692000 | −1.251073000 |
| | | C | −6.315799000 | −3.081307000 | 0.113188000 |
| | | C | 4.579425000 | −1.160608000 | −0.742866000 |
| | | O | 4.177325000 | −1.486946000 | −1.843362000 |
| | | O | 5.840299000 | −1.361667000 | −0.367021000 |
| | | C | 2.808299000 | −1.572763000 | 1.049072000 |
| | | C | 3.647376000 | −2.557566000 | 1.854212000 |
| | | O | 4.440434000 | −3.321477000 | 1.347646000 |
| | | O | 3.452889000 | −2.594386000 | 3.189896000 |
| | | H | 3.095157000 | 3.303783000 | −2.008467000 |
| | | H | 3.648350000 | 3.957850000 | −0.486571000 |
| | | H | 1.982939000 | −1.595437000 | −1.739367000 |
| | | H | 5.024402000 | 0.873012000 | 1.089767000 |
| | | H | 4.091978000 | 0.187337000 | 2.260203000 |
| | | H | 1.610178000 | 5.444719000 | −0.374897000 |
| | | H | −0.777123000 | 5.631454000 | 0.285715000 |
| | | H | 0.447713000 | −3.484555000 | −2.059661000 |
| | | H | −4.979572000 | 3.649952000 | 1.200596000 |
| | | H | −6.480812000 | 1.686656000 | 1.174366000 |
| | | H | −6.735053000 | −0.658229000 | 0.722096000 |
| | | H | −4.143732000 | −4.313778000 | −0.959218000 |
| | | H | −1.815700000 | −4.536950000 | −1.664332000 |
| | | H | −6.076718000 | −4.095693000 | −0.171008000 |
| | | H | −7.333676000 | −2.881513000 | 0.429552000 |
| | | H | 5.879571000 | −1.045918000 | 0.570477000 |
| | | H | 2.258397000 | −2.174493000 | 0.329695000 |
| | | H | 2.069875000 | −1.063891000 | 1.675167000 |
| | | H | 2.774682000 | −1.960809000 | 3.467922000 |
| | Excited state | C | 2.949590000 | 0.314269000 | −0.290861000 |
| | | C | 3.909301000 | 1.377595000 | −0.304343000 |
| | | C | 3.421533000 | 2.820726000 | −0.315376000 |
| | | C | 1.945627000 | 3.029105000 | −0.140694000 |
| | | C | 1.065109000 | 1.926907000 | −0.147321000 |
| | | C | 1.563315000 | 0.568085000 | −0.235871000 |
| | | C | 0.627395000 | −0.506912000 | −0.253866000 |
| | | C | 1.093310000 | −1.855397000 | −0.372166000 |
| | | N | 2.437061000 | −2.085705000 | −0.484311000 |
| | | C | 3.507710000 | −1.103040000 | −0.276932000 |
| | | C | −0.332102000 | 2.149645000 | −0.040093000 |
| | | C | −1.239998000 | 1.066473000 | −0.056571000 |
| | | C | −0.769216000 | −0.269593000 | −0.170835000 |
| | | C | 1.418717000 | 4.334502000 | 0.001498000 |
| | | C | 0.065427000 | 4.571312000 | 0.109812000 |
| | | C | −0.870630000 | 3.488090000 | 0.085239000 |
| | | C | −1.707365000 | −1.343838000 | −0.198284000 |
| | | C | −1.227200000 | −2.678954000 | −0.318372000 |
| | | C | 0.181075000 | −2.906437000 | −0.405525000 |
| | | N | −2.181843000 | 3.748714000 | 0.180008000 |
| | | C | −3.057796000 | 2.694924000 | 0.154097000 |
| | | C | −2.623063000 | 1.338153000 | 0.037119000 |
| | | C | −3.552376000 | 0.274839000 | 0.012481000 |
| | | C | −3.101669000 | −1.085852000 | −0.107881000 |
| | | O | 5.141183000 | 1.167074000 | −0.349111000 |
| | | C | −4.450206000 | 2.932972000 | 0.244269000 |
| | | C | −5.365560000 | 1.883771000 | 0.218870000 |
| | | C | −4.920962000 | 0.551857000 | 0.103022000 |
| | | N | −5.811628000 | −0.516226000 | 0.074191000 |
| | | C | −5.475057000 | −1.853381000 | −0.039924000 |
| | | C | −4.030268000 | −2.146896000 | −0.135511000 |
| | | C | −3.538979000 | −3.460882000 | −0.255011000 |
| | | C | −2.174644000 | −3.725055000 | −0.344419000 |
| | | C | −6.472638000 | −2.792586000 | −0.056736000 |
| | | C | 4.464393000 | −1.325892000 | −1.497409000 |
| | | O | 5.402978000 | −2.092787000 | −1.478006000 |
| | | O | 4.105366000 | −0.717743000 | −2.639544000 |
| | | C | 4.324413000 | −1.429230000 | 0.996663000 |
| | | C | 3.547178000 | −1.415319000 | 2.296135000 |
| | | O | 2.369374000 | −1.165934000 | 2.427720000 |
| | | O | 4.280437000 | −1.716181000 | 3.396435000 |
| | | H | 3.989439000 | 3.372976000 | 0.445895000 |
| | | H | 3.751391000 | 3.254990000 | −1.272490000 |
| | | H | 2.735882000 | −3.052076000 | −0.495685000 |
| | | H | 2.110033000 | 5.172806000 | 0.016609000 |
| | | H | −0.324705000 | 5.578531000 | 0.211422000 |
| | | H | 0.540861000 | −3.927091000 | −0.497623000 |
| | | H | −4.794634000 | 3.957498000 | 0.333099000 |
| | | H | −6.428971000 | 2.091860000 | 0.287453000 |
| | | H | −6.798129000 | −0.299779000 | 0.143097000 |
| | | H | −4.227542000 | −4.297409000 | −0.279034000 |
| | | H | −1.833014000 | −4.751340000 | −0.435295000 |
| | | H | −6.259390000 | −3.847238000 | −0.142075000 |
| | | H | −7.512302000 | −2.496064000 | 0.016477000 |
| | | H | 3.363244000 | −0.112942000 | −2.454569000 |
| | | H | 5.144367000 | −0.707687000 | 1.066104000 |
| | | H | 4.792276000 | −2.412804000 | 0.874668000 |
| | | H | 5.205193000 | −1.890925000 | 3.161562000 |
| 2,6- | Ground state | C | 2.728354000 | 0.512805000 | −0.317929000 |
| CQDs | | C | 3.541203000 | 1.687153000 | −0.417668000 |
| | | C | 2.868498000 | 3.046558000 | −0.490160000 |
| | | C | 1.380006000 | 3.081833000 | −0.294322000 |
| | | C | 0.647714000 | 1.883762000 | −0.224612000 |
| | | C | 1.319049000 | 0.592140000 | −0.257083000 |
| | | C | 0.515450000 | −0.591319000 | −0.237045000 |
| | | C | 1.184481000 | −1.890175000 | −0.307539000 |
| | | C | 2.541980000 | −1.989714000 | −0.272251000 |
| | | C | 3.482095000 | −0.809799000 | −0.191546000 |
| | | C | −0.769625000 | 1.965784000 | −0.098535000 |
| | | C | −1.543434000 | 0.775591000 | −0.066404000 |
| | | C | −0.889453000 | −0.503626000 | −0.153551000 |
| | | C | 0.744200000 | 4.322535000 | −0.199698000 |
| | | C | −0.635438000 | 4.440540000 | −0.040001000 |
| | | C | −1.433327000 | 3.239263000 | −0.013909000 |
| | | C | −1.676450000 | −1.694096000 | −0.142442000 |
| | | C | −1.029226000 | −2.989114000 | −0.276306000 |
| | | C | 0.343961000 | −3.085759000 | −0.397666000 |
| | | C | −2.834049000 | 3.283481000 | 0.082173000 |
| | | C | −3.605507000 | 2.122576000 | 0.113211000 |
| | | C | −2.951740000 | 0.848600000 | 0.037267000 |
| | | C | −3.735416000 | −0.335316000 | 0.057020000 |
| | | C | −3.069116000 | −1.599754000 | −0.040494000 |
| | | O | 4.790765000 | 1.645731000 | −0.483724000 |
| | | C | −5.033034000 | 2.162714000 | 0.213096000 |
| | | C | −5.776095000 | 1.016030000 | 0.237712000 |
| | | C | −5.144965000 | −0.263649000 | 0.156817000 |
| | | C | −5.881044000 | −1.478770000 | 0.167222000 |
| | | C | −5.227818000 | −2.672402000 | 0.069670000 |
| | | N | −3.862996000 | −2.750730000 | −0.037516000 |
| | | C | −3.246802000 | −4.008777000 | −0.150996000 |
| | | C | −1.911775000 | −4.131783000 | −0.270711000 |
| | | N | −1.219674000 | 5.680089000 | 0.031297000 |
| | | N | 0.993520000 | −4.311161000 | −0.535002000 |
| | | C | 4.438800000 | −1.000475000 | −1.403470000 |
| | | O | 5.472305000 | −1.639935000 | −1.372600000 |
| | | O | 3.982524000 | −0.521419000 | −2.578881000 |
| | | C | 4.373832000 | −0.897114000 | 1.079677000 |
| | | C | 3.648774000 | −0.863477000 | 2.407062000 |
| | | O | 2.469925000 | −0.659618000 | 2.593440000 |
| | | O | 4.449628000 | −1.071601000 | 3.488624000 |
| | | H | 3.365979000 | 3.707816000 | 0.229733000 |
| | | H | 3.109883000 | 3.472776000 | −1.474846000 |
| | | H | 3.027330000 | −2.959320000 | −0.244748000 |
| | | H | 1.346442000 | 5.226652000 | −0.236432000 |
| | | H | −3.354768000 | 4.234799000 | 0.113066000 |
| | | H | −5.520852000 | 3.131452000 | 0.271372000 |
| | | H | −6.857670000 | 1.058876000 | 0.315249000 |
| | | H | −6.961224000 | −1.470729000 | 0.246748000 |
| | | H | −5.745368000 | −3.622951000 | 0.067528000 |
| | | H | −3.924702000 | −4.850919000 | −0.132450000 |
| | | H | −1.522117000 | −5.140632000 | −0.336025000 |
| | | H | −2.101037000 | 5.772817000 | 0.513546000 |
| | | H | −0.598914000 | 6.461905000 | 0.187612000 |
| | | H | 1.841235000 | −4.256265000 | −1.087181000 |
| | | H | 0.415045000 | −5.068054000 | −0.873948000 |
| | | H | 3.177211000 | −0.001751000 | −2.388698000 |
| | | H | 5.089411000 | −0.068133000 | 1.063695000 |
| | | H | 4.966605000 | −1.817311000 | 1.031368000 |
| | | H | 5.365887000 | −1.221176000 | 3.208557000 |
| | Excited state | C | 2.722476000 | 0.515985000 | −0.277185000 |
| | | C | 3.549629000 | 1.689494000 | −0.388364000 |
| | | C | 2.902436000 | 3.053762000 | −0.279209000 |
| | | C | 1.404271000 | 3.100718000 | −0.191375000 |
| | | C | 0.653014000 | 1.898657000 | −0.159773000 |
| | | C | 1.321225000 | 0.606147000 | −0.205225000 |
| | | C | 0.508632000 | −0.585859000 | −0.204840000 |
| | | C | 1.153305000 | −1.886215000 | −0.292034000 |
| | | C | 2.526233000 | −1.982325000 | −0.255873000 |
| | | C | 3.458085000 | −0.810492000 | −0.231767000 |
| | | C | −0.762095000 | 1.974981000 | −0.081580000 |
| | | C | −1.543568000 | 0.782883000 | −0.062541000 |
| | | C | −0.897173000 | −0.503165000 | −0.127645000 |
| | | C | 0.771176000 | 4.334581000 | −0.122205000 |
| | | C | −0.622333000 | 4.443288000 | −0.013980000 |
| | | C | −1.428033000 | 3.248883000 | −0.019528000 |
| | | C | −1.670306000 | −1.698708000 | −0.102581000 |
| | | C | −1.060286000 | −2.998491000 | −0.205145000 |
| | | C | 0.336670000 | −3.074751000 | −0.388140000 |
| | | C | −2.831186000 | 3.298657000 | 0.027952000 |
| | | C | −3.613319000 | 2.121609000 | 0.056722000 |
| | | C | −2.960472000 | 0.858285000 | 0.014606000 |
| | | C | −3.747900000 | −0.334319000 | 0.045260000 |
| | | C | −3.097267000 | −1.599468000 | −0.007088000 |
| | | O | 4.784101000 | 1.621925000 | −0.564529000 |
| | | C | −5.039077000 | 2.171742000 | 0.127493000 |
| | | C | −5.798321000 | 1.033760000 | 0.164142000 |
| | | C | −5.163498000 | −0.249062000 | 0.124087000 |
| | | C | −5.891418000 | −1.462769000 | 0.158172000 |
| | | C | −5.239715000 | −2.662719000 | 0.110754000 |
| | | N | −3.868675000 | −2.751734000 | 0.027344000 |
| | | C | −3.272323000 | −4.012866000 | −0.017054000 |
| | | C | −1.898351000 | −4.139995000 | −0.139582000 |
| | | N | −1.212960000 | 5.684386000 | 0.044936000 |
| | | N | 0.940373000 | −4.296392000 | −0.595266000 |
| | | C | 4.333665000 | −1.001448000 | −1.516554000 |
| | | O | 5.354851000 | −1.654990000 | −1.548932000 |
| | | O | 3.817076000 | −0.497087000 | −2.652351000 |
| | | C | 4.435601000 | −0.923886000 | 0.974038000 |
| | | C | 3.787182000 | −0.915101000 | 2.342008000 |
| | | O | 2.605609000 | −0.804182000 | 2.582811000 |
| | | O | 4.656961000 | −1.040458000 | 3.377672000 |
| | | H | 3.334612000 | 3.533738000 | 0.610260000 |
| | | H | 3.253844000 | 3.661884000 | −1.122114000 |
| | | H | 3.009799000 | −2.951744000 | −0.202796000 |
| | | H | 1.369142000 | 5.242291000 | −0.131631000 |
| | | H | −3.351777000 | 4.249944000 | 0.020296000 |
| | | H | −5.519196000 | 3.146331000 | 0.156870000 |
| | | H | −6.880441000 | 1.081966000 | 0.222501000 |
| | | H | −6.973377000 | −1.456086000 | 0.220711000 |
| | | H | −5.759406000 | −3.611193000 | 0.134083000 |
| | | H | −3.953416000 | −4.847576000 | 0.046786000 |
| | | H | −1.491917000 | −5.142857000 | −0.137114000 |
| | | H | −2.099345000 | 5.761036000 | 0.523470000 |
| | | H | −0.596269000 | 6.461326000 | 0.240026000 |
| | | H | 1.826023000 | −4.279011000 | −1.082440000 |
| | | H | 0.339838000 | −5.029118000 | −0.947945000 |
| | | H | 3.034273000 | 0.038179000 | −2.425388000 |
| | | H | 5.148807000 | −0.095311000 | 0.916928000 |
| | | H | 5.018774000 | −1.844762000 | 0.868717000 |
| | | H | 5.568334000 | −1.125283000 | 3.056515000 |

In sum, we found that the high degree of observed tumor-specificity is not unique to LAAM TC-CQDs, but a class of CQDs bearing paired α-carboxyl and amino groups. It appears that the anchorage of α-carboxyl and amino groups camouflages CQDs as large amino acids, which trigger a LAT1-mediated tumor-specific interaction. We designate CQDs bearing paired α-carboxyl and amino groups as “large amino acid mimicking CQDs” or LAAM CQDs. Structurally, the location and number of carboxyl and amino groups determine the tumor-specificity and cell penetration efficiency of LAAM TC-CQDs.

### Pharmaceutical Compositions, Combinations, and Other Related Uses

The compounds and CQDs disclosed herein can be used as pharmaceuticals, for imaging or for therapy. When so used, they may be admixed with one or more pharmaceutically acceptable carriers or excipients.

Accordingly, the invention provides for a pharmaceutical composition comprising a compound, particle or CQD(s) as described herein, admixed with at least one pharmaceutically acceptable carrier or excipient. Optionally, the pharmaceutical composition comprises at least two pharmaceutically acceptable carriers or excipients. Suitable excipients and carriers for use in pharmaceutical compositions of these compounds for either therapeutic uses or imaging uses are known in the art. Because the compounds and CQDs of the invention are generally water soluble, they are typically formulated in aqueous solution/suspension, e.g., in sterile water, or an isotonic aqueous solution containing saline, glucose, lactose, and the like. For imaging purposes, they are typically administered parenterally, and are formulated accordingly.

In yet another aspect, the present disclosure provides for a use of the compounds and particles thereof as described above for the manufacture of a medicament.

In one aspect, the invention provides pharmaceutical compositions comprising a compound or CQDs as described herein for use in therapy. In some embodiments, the use in therapy for is use for treating a tumor or cancer selected from the group consisting of sarcoma, epidermoid cancer, fibrosarcoma, cervical cancer, gastric carcinoma, skin cancer, leukemia, lymphoma, lung cancer, non-small cell lung cancer, colon cancer, melanoma, ovarian cancer, renal cancer, prostate cancer, breast cancer, liver cancer, head and neck cancers, pancreatic cancer, bladder cancer, esophagus cancer, tongue cancer, and cutaneous malignant melanoma.

### Formulations

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds are administered in a pharmacological composition, it is contemplated that the compounds can be formulated in admixture with a pharmaceutically acceptable excipient and/or carrier. For example, contemplated compounds can be administered orally as neutral compounds or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

One of ordinary skill in the art may modify the formulations within the teachings of the specification to provide numerous formulations for a particular route of administration. In particular, the compounds may be modified to render them more soluble in water or other vehicle. It is also well within the ordinary skill of the art to modify the route of administration and dosage regimen of a particular compound in order to manage the pharmacokinetics of the present compounds for maximum beneficial effect in a patient.

### Drug Combinations

The methods of the embodiments comprise administering an effective amount of at least one exemplary compound of the present disclosure; optionally the compound may be administered in combination with one or more additional therapeutic agents, particularly therapeutic agents known to be useful for treating a condition or disease afflicting the subject to be treated by the methods herein.

The additional active ingredients may be administered in a separate pharmaceutical composition from at least one exemplary compound of the present disclosure or may be included with at least one exemplary compound of the present disclosure in a single pharmaceutical composition. The additional active ingredients may be administered simultaneously with, prior to, or after administration of at least one exemplary compound of the present disclosure.

### Methods of Using the Exemplary Compounds and Pharmaceutical Compositions Thereof

To practice the method of the present invention, compounds having formula and pharmaceutical compositions thereof may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

Where the carbon quantum dots of the invention are used for imaging a tumor or tissue in a subject, they are typically administered parenterally, and often by intravenous injection or infusion. Suitable formulations and methods for such administration are known in the art.

A sterile injectable composition, such as a sterile injectable aqueous or oleaginous suspension, may be formulated according to techniques known in the art using suitable dispersing or wetting agents and suspending agents if such are needed. The sterile injectable preparation may also be a sterile injectable solution or suspension in a non-toxic parenterally acceptable diluent or solvent. The preferred carrier for the CQDs of the invention is sterile water, which may be modified by addition of suitable buffers and other excipients. Among the acceptable carriers that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

In addition, the compounds and compositions of the invention may be administered alone or in combination with other therapeutic agents, e.g., anticancer agents, for the treatment of various cancers or tumors that may be treated with the compounds and compositions of the invention. Combination therapies according to the present invention comprise the administration of at least one exemplary compound of the present disclosure and at least one other pharmaceutically active ingredient. The compound of the invention and the other pharmaceutically active agents may be administered separately or together. The amounts of the active ingredient(s) and pharmaceutically active agent(s) and the relative timings of administration will be selected in order to achieve the desired combined therapeutic effect.

### Examples

Materials and Reagents

1,4-diaminoanthraquinone (1,4-DAAQ, 90%), 1,5-diaminoanthraquinone (1,5-DAAQ, 90%), 2,6-diaminoanthraquinone (2,6-DAAQ, 90%), 1,4,5,8-tetraminoanthraquinone (TAAQ, 90%) and citric acid (CA, 99.8%) were purchased from Sigma-Aldrich. Dichloromethane (99.5%) and methanol (99.5%) were provided by Beijing Chemical Reagent Ltd. The cell counting kit-8 (CCK-8) was supplied by Dojindo Laboratories (Japan). The water used throughout all the experiments was purified through a Millipore system (ULUPURE, Chengdu, China). All the BALB/c female mice were purchased from Beijing Laboratory Animal Research Center, and the weight of each mouse was 18-20 g.

### Synthesis of TC-CQDs

0.04 g CA and 0.03 g TAAQ were firstly mixed in 10 mL pure water, and then the solution was transferred into poly(tetrafluoroethylene)-lined autoclaves (25 mL). After heating at 180° C. in oven for 2 h and cooling down to room temperature naturally, blue suspension was obtained. The crude product was then purified with a silica column chromatography using mixtures of dichloromethane and methanol (10:1) as eluents. After removing solvents and further drying under vacuum, the purified TC-CQDs could be finally obtained in 25 wt % yields.

### Characterization

Transmission electron micrographs (TEM) were taken on a JEOL JEM 2100 transmission electron microscope (FEI). Atomic force microscopic (AFM) images were obtained by MultiMode V SPM (VEECO). X-ray diffraction (XRD) patterns were carried out with an X-ray diffraction using Cu-Kα radiation (XRD, PANalytical X'Pert Pro MPD). The Raman spectra were measured using Laser Confocal Micro-Raman Spectroscopy (LabRAM Aramis). X-ray photoelectron spectroscopy (XPS) was performed with an ESCALab 250Xi electron spectrometer from VG Scientific using 300 W Al Kα radiation. UV-vis absorption and fluorescence spectra were recorded on UV-2600 spectrophotometer and a PerkinElmer-LS55 fluorescence spectrometer, respectively. The Fourier transform infrared spectroscopy (FT-IR) were measured using a Nicolet 380 spectrograph. The ¹³C NMR spectra were recorded at 400 MHz on a Bruker Advance III spectrometer in CD₃OD, with chemical shift values in parts per million.

### Computational Methods

The ground state and the first excited state of one fluorescent unit of CQDs were obtained from theoretical calculation with density function theory (B3LYP/6-31G(d)). The geometric parameters of the ground state were optimized and verified at B3LYP/6-31G(d) level and the geometric parameters of the first excited state were optimized with TD-B3LYP/6-31G(d).

Cell culture and materials. Cells except BCSCs were cultured in DMEM or RPMI medium (Invitrogen) supplemented with 10% fetal bovine serum (Invitrogen), 100 units ml-1 penicillin, and 100 μg ml-1 streptomycin (Invitrogen) in a 37° C. incubator containing 5% CO₂. Human BCSCs were enriched and cultured as previously reported 34. All chemicals were purchased from Sigma-Aldrich unless otherwise noted. The antibody against LAT1 was purchased from Novus Biologicals.

CQD synthesis. LAAM TC-CQDs were synthesized by CA with TAAQ in aqueous solution that were treated at 180° C. for 2 h. The resulting LAAM TC-CQDs were purified using silica gel column chromatography.

Photothermal effects for LAAM TC-CQDs in aqueous solution. To evaluate the photothermal effects in aqueous solution, LAAM TC-CQDs aqueous solution at various concentrations (0-10 μg/mL) were exposed to 650 nm laser irradiation (0.5 W/cm²) with the illumination direction from the top to the bottom of the cuvette for 5 min. An equivalent amount of pure water with the same laser irradiation was used as a control. Real-time temperature was recorded every 30 s by an infrared thermal camera.

Given their efficient NIR absorption features, the photothermal performance of TC-CQDs aqueous solution were investigated. The temperatures of TC-CQDs aqueous solution at various concentrations (0-10 μg/mL) are monitored under continuous laser irradiation (650 nm, 0.5 W/cm²) with an infrared thermal camera, as displayed in FIG. 13a. No obvious temperature rise is observed in the control sample of pure water, while TC-CQDs (10 μg/mL) could quickly trigger the increase of temperature and display a concentration-dependent hyperthermia (above 42° C.) during a short photoirradiation (5 min), leading to an irreversible damage to tumor cells^{7,8}. To further study the photothermal stability and transduction efficiency of TC-CQDs, TC-CQDs aqueous solution (10 μg/mL, 3 mL) is continuously illuminated by a 650 nm laser (0.5 W/cm²) until it reaches a steady-state temperature, at which point the laser is stopped and the suspension is allowed to cool naturally. The temperature change during the heating-cooling process is monitored for five cycles to derive a heat generation-dissipation curve as shown in FIG. 13b. An almost equal temperature elevation of 25.6° C. occurred during each laser ON/OFF cycle, suggesting that TC-CQDs possess preferable photothermal stability compared with extensively used organic dyes and inorganic nanomaterials with surface plasmon resonances.³⁻¹⁵ The photothermal conversion efficiency (η) of TC-CQDs was calculated using the following equations 1.

$$η = \frac{hS ( {T}_{Max} - {T}_{Surr} ) - {Q}_{Dis}}{I ( 1 - {10}^{- {A}_{650}} )} \qquad ( 1 )$$

Where h is heat transfer coefficient, S is the surface area of the container, T_{Max} is the maximum system temperature, T_{Surr} is ambient temperature of the surroundings, Q_{Dis} is the baseline energy inputted by the sample cell, I is incident laser power and A₆₅₀ is the absorbance of the TC-CQDs at wavelength of 650 nm. (T_{Max}−T_{Surr}) is 25.6° C. according to FIG. 13c, I is 0.5 W/cm2 and A₆₅₀ is 1.4908. Q_{Dis} expresses heat dissipated from light absorbed by the quartz sample cell itself, and it was measured independently to be 37.2 mW using a quartz cuvette cell containing pure water without TC-CQDs. Thus, only hS remains unknown for calculating η.

In order to get hS, a dimensionless driving force temperature (θ) and a sample system time constant τₛ are introduced.

$$θ = \frac{T - {T}_{Surr}}{{T}_{Max} - {T}_{Surr}} \qquad ( 2 )$$

$${τ}_{s} = \frac{{\sum }_{i} {m}_{i} {C}_{p , i}}{hS} \qquad ( 3 )$$

Where m and Cₚ are the mass and heat capacity of water, respectively. When θ and τₛ are substituted into the following total energy balance equation (4) for the system, Equation (5) is yielded.

$${\sum }_{i} {m}_{i} {C}_{p , i} \frac{dT}{dt} = {Q}_{NC} + {Q}_{Dis} - {Q}_{Surr} \qquad ( 4 )$$

Where Q_{NC} is the energy inputted by IR780/CQDs-FA, and Q_{Surr} is heat conduction away from the system surface by air.

$$\frac{d θ}{dt} = \frac{1}{{τ}_{s}} [ \frac{{Q}_{NC} + {Q}_{Dis}}{hS ( {T}_{Max} - {T}_{Surr} )} - θ ] \qquad ( 5 )$$

At the cooling stage of the aqueous dispersion of the IR780/CQDs-FA, the light source was shut off, and Q_{NC}+Q_{Dis}=0.

$$dt = - {τ}_{s} \frac{d θ}{θ} \qquad ( 6 )$$

$$t = - {τ}_{s} ln θ \qquad ( 7 )$$

Therefore, time constant for heat transfer from the system is determined to be τₛ=83.86 s by applying the linear time data from the cooling period (after 300 s) vs negative natural logarithm of driving force temperature (FIG. 13d). In addition, m is 0.3 g and Cₚ is 4.2 J/g. Thus, according to equation (3), hS is deduced to be 16.1 mW/° C. Substituting 16.1 mW/° C. of hS into equation (1), the η of TC-CQDs can be calculated to be 77.4%, which is comparable to that of the previously reported photothermal conversion agents (PTCAs), such as Au bellflowers (74%), Au nanocages (64%), Au nanorods (55%), dopamine-melanin nanospheres (40%), carbon dots (38.5%), Cu9S5 nanocrystals (25.7%), Cu2-xSe nanocrystals (22%), and so on (Table 2)^{s3-s15}.

Flow cytometry. MDA-MB-231 SP cells were sorted according to our previously reported methods²². Briefly, cells harvested at about 85% confluence were resuspended in RPMI-1640 supplemented with 2% fetal bovine serum (FBS) at a density of 1×10⁶ cells/mL and incubated with Hoechst 33342 at a concentration of 5 μg/mL at 37° C. After 90 min, the cells were suspended in cold PBS at a concentration of 1×10⁶ cells/mL, filtered through a 40 μm cell strainer to remove cell aggregates, stained with 1 μg/mL propidium iodide (PI), and analyzed and sorted using a FACSDiva (Becton Dickinson, USA). To characterize SP cells, freshly sorted SP cells were suspended in cold PBS and stained with antihuman CD44-FITC and CD24-PE or their appropriate isotype controls on ice for 30 min. The cells were washed 3 times with cold PBS, resuspended in 400 mL of cold PBS, and analyzed by a FACScan flow cytometer (BD, Ann Arbor, Mich.). To characterize the interaction between CQDs and cells, selected cells were placed on a 6-well plate and treated with LAAM TC-CQDs at 10 μg/mL. Cells without CQDs treatment were used as controls. After 12 h, the cells were washed with fresh medium, trypsinized, resuspended in PBS with 0.5% FBS, and analyzed using a BD FACSCalibur (BD Biosciences, USA). Data were analyzed using FlowJo 7.6.

Confocal laser scanning microscope imaging. To determine the update of CQDs, cells were placed on glass chamber slides and treated with LAAM TC-CQDs at 10 μg/mL. After 8 h, cells were washed with PBS twice, fixed with 4% polyformaldehyde for 30 min at room temperature, followed by addition of DAPI for cell nuclei staining. Finally, the slides were washed three times, fixed, sealed with cover glasses, and imaged with excitation/emission: 561/700 using a confocal laser scanning microscopy (Leica TCS-SP8, Germany). To determine the effects of Leu, Phe, Gly or BCH on CQD uptake, HeLa cells were placed and treated with Leu, Phe, Gly or BCH. Four hours later, LAAM TC-CQDs were added to cells. After an additional 8 h, the cells were washed, fixed, and imaged.

PA imaging of LAAM TC-CQDs. LAAM TC-CQDs with different concentrations (0, 2, 4, 6, 8 and 10 μg/mL) were added to agarose tubes (37° C.) and subjected to scanning using a PA imaging instrument (mode: iTheraMedical Co. MOST inVision 128; excitation wavelength ranged from 640-840 nm with 5 nm interval). PA signal was recorded.

Cellular toxicity tests. Cells were plated into a 96-well plate at a density of 1×10⁴ cells per well and treated with LAAM TC-CQDs, TPTC, or TPTC/TC-CQDs at various concentrations. After 12 h, the cultured medium was removed, and washed with PBS. One hundred microliters of fresh medium containing 10 μL CCK-8 (Sigma) was added to each well. After 2 h incubation at 37° C., the absorbance at 450 nm was measured using a microplate reader.

NIR FL imaging of LAAM TC-CQDs in vivo. Female BALB/c nude mice were maintained in a sterile environment and used for in vivo imaging of LAAM TC-CQDs. This project was approved by the Animal Use Committee at Beijing Normal University and Yale IACUC. For establishment of mice bearing HeLa tumors, 2×10⁶ HeLa cells were prepared and subcutaneously inoculated into female BALB/c mice. When the tumor volumes reached about ˜100 mm³, LAAM TC-CQDs (5 mg/kg) was intravenously administered into mice. NIR FL images were captured at 1, 2, 4, 6, 8 and 10 h using an animal optical imaging system (IVIS Lumina III, Caliper Life Sciences).

PA imaging of LAAM TC-CQDs in vivo. Prior to intravenous injection of LAAM TC-CQDs, pre-contrast data with excitation wavelength from 640 to 840 nm were obtained. Tumor-bearing mice were treated with LAAM TC-CQDs at 5 mg/kg. Post-contrast data were acquired at 2, 4, 6, 8 and 10 h after injection. PA images were reconstructed using data acquired from all 128 transducers at each view through a modified back-projection algorithm.

Therapeutic evaluation in tumor-bearing mice. HeLa cells were subcutaneously inoculated into female BALB/c mice. When the tumor volumes reached ˜100 mm³, the mice were intravenously administered with, saline, TPTC or TPTC/TC-CQDs (n=5). Changes in tumor volume and body weight were monitored daily. The volume of the tumor was calculated according to the following formula: V=D×d²/2 (where D and d are the longest and shortest diameters of tumor, respectively, measured using a vernier caliper). Relative tumor volumes were calculated as V/V₀(V₀ is the initial tumor volume when the treatment was started).

Toxicity evaluation in mice. Female BALB/c mice were intravenously administered with TPTC (10 mg/kg, 0.05 mL per mice) or TPTC/TC-CQDs (10 mg/kg for TPTC, 0.05 mL per mice) (n=5). Mice treated with saline were used as controls. At selected time points, blood samples were collected in heparinized microhematocrit tubes and centrifuged at a speed of 3000 rpm for 10 min. At the end of the study, mice were euthanized. Major organs, including the heart, liver, spleen, kidney and lung were excised, fixed in formalin, and analyzed.

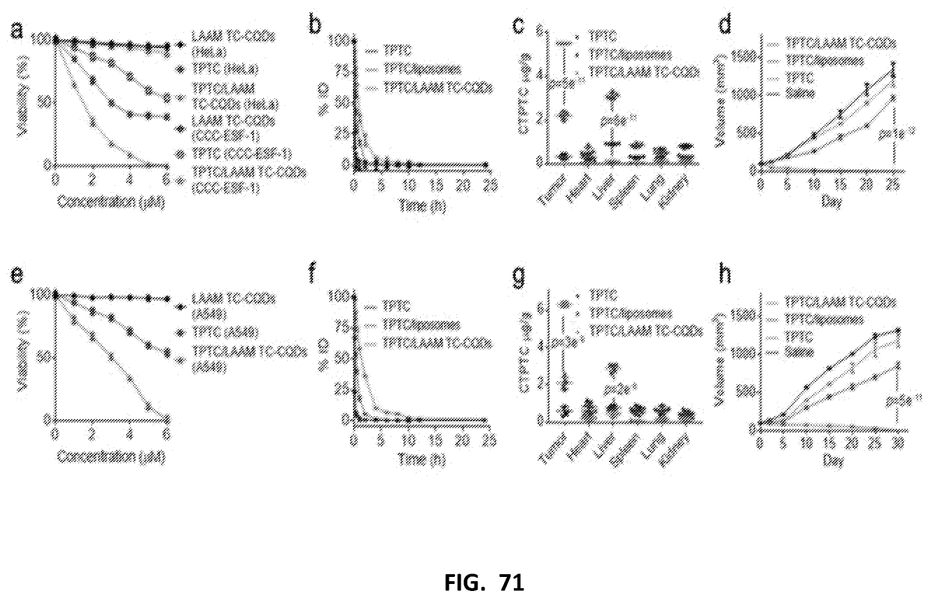

TPTC/LAAM TC-CQDs for treatment of HeLa tumors. Delivery via LAAM TC-CQDs significantly enhanced the toxicity of TPTC to HeLa cells but reduced its toxicity to non-cancerous cells (see FIG. 71a). At a concentration equivalent to 5 μg/mL TPTC, TPTC/LAAM TC-CQDs killed all HeLa cells but did not exhibit toxicity to non-cancerous CCC-ESF-1 cells. We determined the pharmacokinetics of TPTC/LAAM TC-CQDs in mice bearing HeLa tumours. For comparison, TPTC-loaded liposomes, designated as TPTC/liposomes, were synthesized using HSPC:Cholesterol:PEG 2000-DSPE (56:39:5 molar ratio). The same lipid composition was used in several FDA-approved liposomal formulations, including Doxil© 38. TPTC/LAAM TC-CQDs and control free TPTC or TPTC/liposomes were administered intravenously at a dose equivalent to 2 mg/kg TPTC. The blood was collected at various time points. TPTC in the plasma was quantified by high performance liquid chromatography (HPLC). As shown in FIG. 71b, delivery via LAAM TC-CQDs significantly enhanced the blood circulation and bioavailability of TPTC, with the half-life of TPTC increasing from 5.4 to 40.1 min and the area under the curve (AUC) increasing from 13.4 to 138.1. In contrast, the half-life and AUC for TPTC/liposomes were 11.8 min and 55.1, respectively (FIG. 71b).

Next, we examined the biodistribution of TPTC in a second cohort of mice that received the same treatment. As expected, free TPTC was widely distributed over the body without specificity. By contrast, TPTC/LAAM TC-CQDs exhibited a kinetics similar to that of LAAM TC-CQDs. By 8 h, TPTC could only be detected in tumors. Quantification based on TPTC FL intensity showed that delivery via LAAM TC-CQDs increased the concentration of TPTC in tumors by 16.7-fold while significantly reducing the accumulation of TPTC in other organs. The amount of LAAM TC-CQDs accumulated in the tumors accounted for 15.3% of the total injection dose. In comparison, a significant amount of TPTC/liposomes were found in the liver. Delivery via the liposomes increased the concentration of TPTC in tumors by 6.7-fold (FIG. 71c).

We also assessed TPTC/LAAM TC-CQDs for treatment of HeLa tumors. When tumor volumes reached ˜100 mm³, the mice were randomly grouped and received intravenous administration of either saline, TPTC (2 mg/kg), TPTC/liposomes or TPTC/LAAM TC-CQDs (both at a dose equivalent to 2 mg/kg TPTC) at days 1, 4, 8, and 12. Fifteen days later, tumors in the mice receiving TPTC/LAAM TC-CQDs treatment shrank to an undetectable level. In contrast, tumors in the mice receiving free TPTC or TPTC/liposomes continuously grew, although in rates that were slower than that of control mice (FIG. 71d). All the mice treated with TPTC/LAAM TC-CQDs survived over 50 days. However, the median survivals for mice treated with saline, TPTC and TPTC/liposomes were limited to 25, 30, and 35 days, respectively.

We repeated the characterization studies using A549 tumours. Experiments were carried out according to the same procedures as described above. Similar to the findings in HeLa tumors, we found that delivery via LAAM TC-CQDs significantly enhanced the toxicity of TPTC to A549 cells (FIG. 71e), increased the half-life and AUC in blood circulation of TPTC by 5.5-fold (half-life went from 5.9 min to 32.5 min) and 13.9-fold (AUC went from 15.9 to 221.5), respectively (FIG. 71f). It also increased the accumulation of TPTC in tumours by 10.3-fold while reducing TPTC in other organs (FIG. 71h), and markedly inhibited tumor progression and prolonged the survival of tumor-bearing mice (FIG. 71h). The overall tumor delivery efficiency was 15.8%. Consistently, treatment with TPTC/liposomes failed to achieve a comparable degree of inhibitory effects or therapeutic benefits as those achieved by addition of LAAM TC-CQDs (FIG. 71f-h).

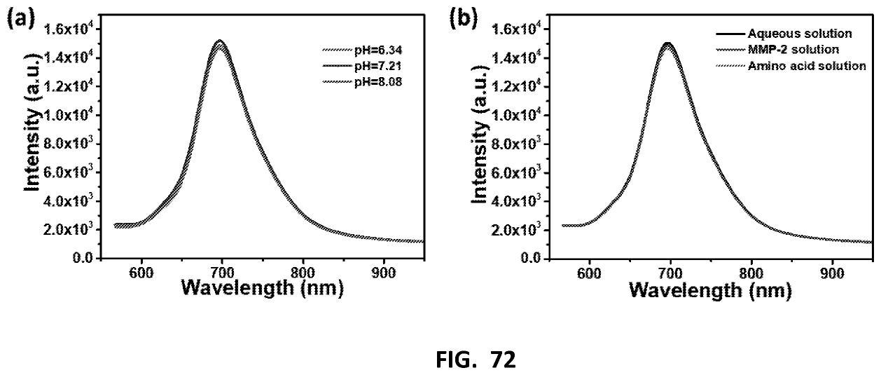

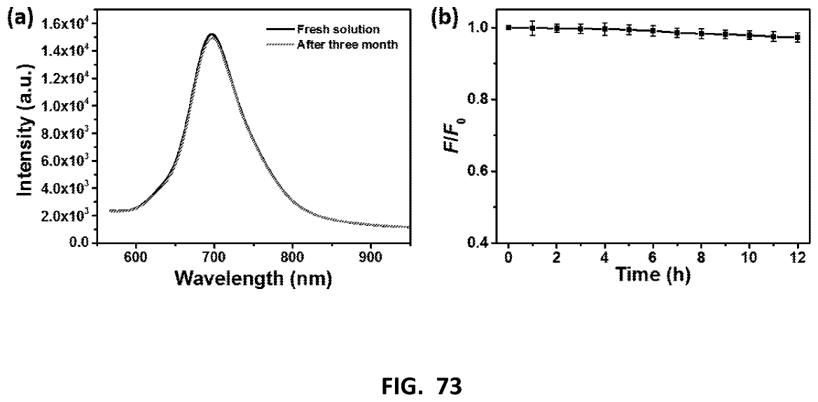

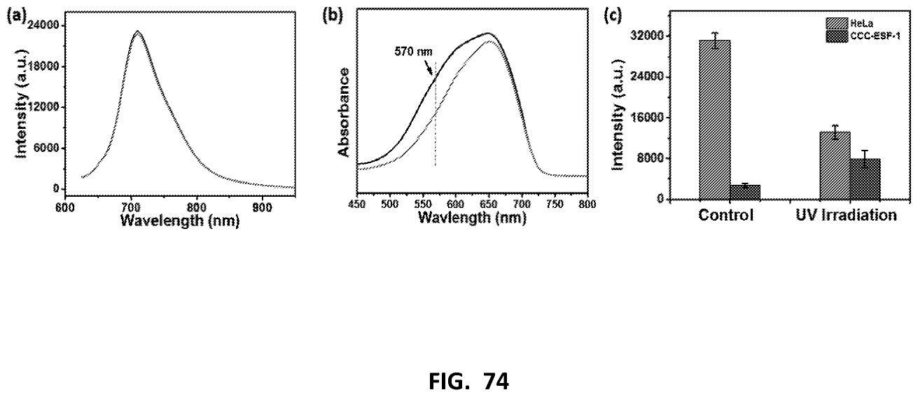

Characterizing the stability of LAAM TC-CQDs for cancer imaging. The tumour microenvironment is acidic (pH ranges from 6.2 to 6.9), and is further complicated by presence of various molecules, such as proteases and amino acids^{28,29}. Therefore, we characterized the FL stability of LAAM TC-CQDs in pHs ranging from 6 to 8 and in presence of amino acids or matrix metalloproteinase-2 (MMP-2). We found that the FL emission spectrum of LAAM TC-CQDs remained unchanged in all the tested conditions (FIG. 72).

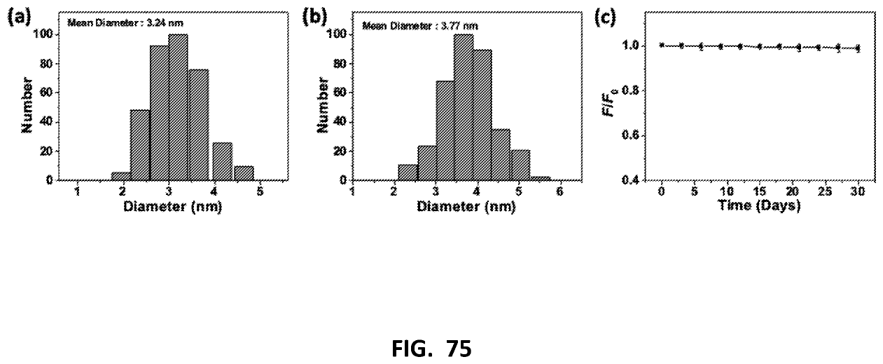

Next, we characterized the photostability of LAAM TC-CQDs after long time storage or irradiation. Results in FIGS. 75 and 76 showed that none of the tested conditions, including storage at room temperature for 3 months, continuous irradiation with a 500 W xenon lamp for 12 hours, and continuous UV irradiation at 365 nm for 5 hours, significantly altered the FL emission spectrum. Lastly, we assessed the impact of long-time incubation in blood serum on the size and FL emission of LAAM TC-CQDs. We found that both the diameter and the FL emission intensity of LAAM TC-CQDs did not change over 30 days after incubation in blood serum (FIG. 75).

### Statistical Analysis

All data were collected in triplicate and reported as mean and standard deviation. Comparison of two conditions was evaluated by the unpaired t-test. One-way ANOVA analysis was carried out to determine the statistical significance of treatment related to survival. p<0.05 (*), 0.01 (**), and 0.0001 (****) were considered significant.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

### REFERENCES

1 Siegel, R. L., Miller, K. D. & Jemal, A. Cancer statistics, 2016. CA Cancer J Clin 66, 7-30, doi:10.3322/caac.21332 (2016).
2 Kim, S. M., Faix, P. H. & Schnitzer, J. E. Overcoming key biological barriers to cancer drug delivery and efficacy. Journal of controlled release:official journal of the Controlled Release Society 267, 15-30, doi:10.1016/j.jconrel.2017.09.016 (2017).
3 Tringale, K. R., Pang, J. & Nguyen, Q. T. Image-guided surgery in cancer: A strategy to reduce incidence of positive surgical margins. Wiley Interdiscip Rev Syst Biol Med, doi:10.1002/wsbm.1412 (2018).
4 Wilhelm, S. et al. Analysis of nanoparticle delivery to tumours. Nature Reviews Materials 1, 12 (2016).
Park, K. Facing the Truth about Nanotechnology in Drug Delivery. ACS nano 7, 7442-7447, doi:10.1021/nn404501g (2013).
6 Zhou, J., Atsina, K. B., Himes, B. T., Strohbehn, G. W. & Saltzman, W. M. Novel delivery strategies for glioblastoma. Cancer J18, 89-99, doi:10.1097/PPO.0b013e318244d8ae (2012).
7 Deeken, J. F. & Loscher, W. The blood-brain barrier and cancer: transporters, treatment, and Trojan horses. Clinical cancer research:an official journal of the American Association for Cancer Research 13, 1663-1674, doi: 10.1158/1078-0432.CCR-06-2854 (2007).
8 Nakanishi, T. & Tamai, I. Solute carrier transporters as targets for drug delivery and pharmacological intervention for chemotherapy. J Pharm Sci 100, 3731-3750, doi:10.1002/jps.22576 (2011).
9 Liu, R. et al. GLUT1-mediated selective tumor targeting with fluorine containing platinum(II) glycoconjugates. Oncotarget 8, 39476-39496, doi:10.18632/oncotarget.17073 (2017).
Jin, S. E., Jin, H. E. & Hong, S. S. Targeting L-type amino acid transporter 1 for anticancer therapy: clinical impact from diagnostics to therapeutics. Expert opinion on therapeutic targets 19, 1319-1337, doi:10.1517/14728222.2015.1044975 (2015).
11 Bodoy, S., Fotiadis, D., Stoeger, C., Kanai, Y. & Palacin, M. The small SLC43 family: facilitator system 1 amino acid transporters and the orphan EEG1. Mol Aspects Med 34, 638-645, doi:10.1016/j.mam.2012.12.006 (2013).
12 Hayashi, K. & Anzai, N. Novel therapeutic approaches targeting L-type amino acid transporters for cancer treatment. World J Gastrointest Oncol 9, 21-29, doi:10.4251/wjgo.v9.i1.21 (2017).
13 Wu, W. et al. Aspartate-modified doxorubicin on its N-terminal increases drug accumulation in LAT1-overexpressing tumors. Cancer science 106, 747-756, doi: 10.1111/cas.12672 (2015).
14 Fan, Z., Zhou, S., Garcia, C., Fan, L. & Zhou, J. pH-Responsive fluorescent graphene quantum dots for fluorescence-guided cancer surgery and diagnosis. Nanoscale 9, 4928-4933, doi:10.1039/c7nr00888k (2017).
Yuan, F. et al. Shining carbon dots: Synthesis and biomedical and optoelectronic applications. Nanotoday 11, 565-586 (2016).
16 Peng, J. et al. Graphene quantum dots derived from carbon fibers. Nano Lett 12, 844-849, doi:10.1021/n12038979 (2012).
17 Fan, Z. T. et al. Surrounding media sensitive photoluminescence of boron-doped graphene quantum dots for highly fluorescent dyed crystals, chemical sensing and bioimaging. Carbon 70, 149-156, doi:10.1016/j.carbon.2013.12.085 (2014).
18 Li, S. et al. Exceptionally High Payload of the IR780 Iodide on Folic Acid-Functionalized Graphene Quantum Dots for Targeted Photothermal Therapy. ACS Appl Mater Interfaces 9, 22332-22341, doi:10.1021/acsami.7b07267 (2017).
19 Zhang, M. et al. Facile synthesis of water-soluble, highly fluorescent graphene quantum dots as a robust biological label for stem cells. J Mater Chem 22, 7461-7467, doi:10.1039/c2jm16835a (2012).
Wang, L. V. & Hu, S. Photoacoustic tomography: in vivo imaging from organelles to organs. Science 335, 1458-1462, doi:10.1126/science.1216210 (2012).
21 Weber, J., Beard, P. C. & Bohndiek, S. E. Contrast agents for molecular photoacoustic imaging. Nature methods 13, 639-650, doi: 10.1038/nmeth.3929 (2016).
22 Zhou, J. et al. Activation of the PTEN/mTOR/STAT3 pathway in breast cancer stem-like cells is required for viability and maintenance. Proc Natl Acad Sci USA 104, 16158-16163, doi:0702596104 [pii] 10.1073/pnas.0702596104 (2007).
23 Li, S. H. et al. Exceptionally High Payload of the IR780 Iodide on Folic Acid-Functionalized Graphene Quantum Dots for Targeted Photothermal Therapy. Acs Appl Mater Inter 9, 22332-22341, doi:10.1021/acsami.7b07267 (2017).
24 Ren, D., Kratz, F. & Wang, S. W. Engineered drug-protein nanoparticle complexes for folate receptor targeting. Biochem Eng J89, 33-41, doi:10.1016/j.bej.2013.09.008 (2014).
Shang, W. et al. The uptake mechanism and biocompatibility of graphene quantum dots with human neural stem cells. Nanoscale 6, 5799-5806, doi: 10.1039/c3nr06433f (2014).
26 Potocky, T. B., Menon, A. K. & Gellman, S. H. Cytoplasmic and nuclear delivery of a TAT-derived peptide and a beta-peptide after endocytic uptake into HeLa cells. J Biol Chem 278, 50188-50194, doi:10.1074/jbc.M308719200 (2003).
27 Uchino, H. et al. Transport of amino acid-related compounds mediated by L-type amino acid transporter 1 (LAT1): insights into the mechanisms of substrate recognition. Mol Pharmacol 61, 729-737 (2002).
28 Cheung-Ong, K., Giaever, G. & Nislow, C. DNA-damaging agents in cancer chemotherapy: serendipity and chemical biology. Chem Biol 20, 648-659, doi: 10.1016/j.chembiol.2013.04.007 (2013).
29 Geier, E. G. et al. Structure-based ligand discovery for the Large-neutral Amino Acid Transporter 1, LAT-1. Proc Natl Acad Sci USA 110, 5480-5485, doi:10.1073/pnas.1218165110 (2013).
30 Li, G. et al. Permeability of endothelial and astrocyte cocultures: in vitro blood-brain barrier models for drug delivery studies. Ann Biomed Eng 38, 2499-2511, doi: 10.1007/s10439-010-0023-5 (2010).
31 Dewhirst, M. W. & Secomb, T. W. Transport of drugs from blood vessels to tumour tissue. Nature reviews. Cancer 17, 738-750, doi: 10.1038/nrc.2017.93 (2017).
32 Lim, S. Y., Shen, W. & Gao, Z. Q. Carbon quantum dots and their applications. Chem Soc Rev 44, 362-381, doi:10.1039/c4cs00269e (2015).
33 Mabray, M. C., Barajas, R. F., Jr. & Cha, S. Modern brain tumor imaging. Brain Tumor Res Treat 3, 8-23, doi:10.14791/btrt.2015.3.1.8 (2015).
34 Zhou, J. et al. Highly penetrative, drug-loaded nanocarriers improve treatment of glioblastoma. Proc Natl Acad Sci USA 110, 11751-11756 (2013).
[s1] Lal, S., Clare, S. E. & Halas, N. J. Nanoshell-enabled photothermal cancer therapy: impending clinical impact. Acc. Chem. Res. 41, 1842-1851 (2008).
[s2] Lim, Y. T. et al. Biocompatible polymer-nanoparticle-based bimodal imaging contrast agents for the labeling and tracking of dendritic cells. Small 4, 1640-1645 (2008).
[s3] Huang, P. et al. Triphase interface synthesis of plasmonic gold bellflowers as near-infrared light mediated acoustic and thermal theranostics. J. Am. Chem. Soc. 136, 8307-8313 (2014).
[s4] Zeng, J., Goldfeld, D. & Xia, Y. A plasmon-assisted optofluidic (PAOF) system for measuring the photothermal conversion efficiencies of gold nanostructures and controlling an electrical switch. Angew. Chem. Int. Ed 52, 4169-4173 (2013).
[s5] Ayala-Orozco, C. et al. Au nanomatryoshkas as efficient near-infrared photothermal transducers for cancer treatment: benchmarking against nanoshells. ACS Nano 8, 6372-6381 (2014).
[s6] Cole, J. R. et al. Photothermal efficiencies of nanoshells and nanorods for clinical therapeutic applications. J. Phys. Chem. C 113, 12090-12094 (2009).
[s7] Santos, G. M., Zhao, F., Zeng, J. & Shih, W.-C. Characterization of nanoporous gold disks for photothermal light harvesting and light-gated molecular release. Nanoscale 6, 5718-5724 (2014).
[s8] Chen, H. et al. Understanding the photothermal conversion efficiency of gold nanocrystals. Small 6, 2272-2280 (2010).
[s9] Pattani, V. P. & Tunnell, J. W. Nanoparticle-mediated photothermal therapy: a comparative study of heating for different particle types. Lasers Surg. Med. 44, 675-684 (2012).
[s10] Liu, Y. et al. Dopamine-melanin colloidal nanospheres: an efficient near-infrared photothermal therapeutic agent for in vivo cancer therapy. Adv. Mater. 25, 1353-1359 (2012).
[s11] Ge, J. et al. Red-emissive carbon dots for fluorescent, photoacoustic, and thermal theranostics in living Mice. Adv. Mater. 27, 4169-4177 (2015).
[s12] Vankayala, R. et al. Gold nanoshells-mediated bimodal photodynamic and photothermal cancer treatment using ultra-low doses of near infra-red light. Biomaterials 35, 5527-5538 (2014).
[s13] Tian, Q. et al. Hydrophilic Cu₉S₅ nanocrystals: a photothermal agent with a 25.7% heat conversion efficiency for photothermal ablation of cancer cells in vivo. ACS nano 5, 9761-9771 (2011).
[s14] Hessel, C. M. et al. Copper selenide nanocrystals for photothermal therapy. Nano Lett. 11, 2560-2566 (2011).
[s15] Tian, Q. et al. Sub-10 nm Fe₃O₄@Cu₍₂₋ₓ₎S core-shell nanoparticles for dual-modal imaging and photothermal therapy. J. Am. Chem. Soc. 135, 8571-8577 (2013).
[s16] Roper, D. K., Ahn, W. & Hoepfner, M. Microscale heat transfer transduced by surface plasmon resonant gold nanoparticles. J. Phys. Chem. C 111, 3636-3641 (2007).
Gillies, R. J. The tumour microenvironment: causes and consequences of hypoxia and acidity. Introduction. Novartis Found Symp 240, 1-6 (2001).
Vaupel, P., Kallinowski, F. & Okunieff, P. Blood-Flow, Oxygen and Nutrient Supply, and Metabolic Microenvironment of Human-Tumors—a Review. Cancer research 49, 6449-6465 (1989).
Bulbake, U., Doppalapudi, S., Kommineni, N. & Khan, W. Liposomal Formulations in Clinical Use: An Updated Review. Pharmaceutics 9, 1-33, doi:UNSP 12 10.3390/pharmaceutics9020012 (2017).

1. A carbon quantum dot, which:
a) comprises a core structure comprising a fused polycyclic array of 6-membered rings, each of which is aromatic or unsaturated, wherein the polycyclic array of 6-membered rings is substituted with at least one C₁-C₃ alkyl group that is substituted with at least a carboxyl group and an amino group; or
b) comprises a fused polycyclic ring system comprising 6-membered rings, wherein each ring is aromatic or unsaturated, and the polycyclic ring system is substituted with at least one group of Formula A: 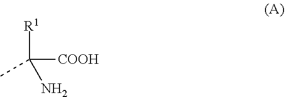
wherein R¹ is H or a C₁-C₃ alkyl group optionally substituted with one or two groups selected from halo, —OH, —OMe, —NH₂, —SMe, —COOH, and —CONH₂, and the dashed bond indicates where said group of Formula A is connected to said fused polycyclic ring system; or
c) comprises a polycyclic aromatic or partially aromatic ring system comprising at least 10 fused 6-membered rings, wherein said polycyclic aromatic or partially aromatic ring system is fused to at least one subunit of Formula B: 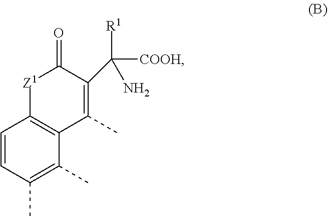
wherein:
R¹ is H or a C₁-C₃ alkyl optionally substituted with one or two groups selected from halo, —OH, —OMe, —NH₂, —SMe, —COOH, and —CONH₂,
Z¹ is NR² or C(R²)₂, where each R² is independently selected from H and C₁-C₃ alkyl, and wherein the dashed bonds indicate where said Formula B is fused to said polycyclic aromatic or partially aromatic ring system; and/or
d) is configured to selectively enter a cell that expresses a large neutral amino acid transporter (LAT1), or a subunit thereof; and/or
e) is configured to selectively enter a tumor or cancer cell,
wherein the percentage of carbon atoms by weight in said carbon quantum dot is 20% or more, preferably 40% or more.

2. The carbon quantum dot of claim 1, which comprises a core structure comprising a fused polycyclic array of 6-membered rings, each of which is aromatic or unsaturated, wherein the polycyclic array of 6-membered rings is substituted with at least one C₁-C₃ alkyl group that is substituted with a carboxyl group and an amino group.

3. The carbon quantum dot of claim 2, wherein the core structure comprises at least 5 fused 6-membered aromatic or unsaturated core rings.

4-6. (canceled)

7. The carbon quantum dot of claim 1, which comprises a fused polycyclic ring system comprising 6-membered rings, wherein each ring is aromatic or unsaturated, and the polycyclic ring system is substituted with at least one group of Formula A: 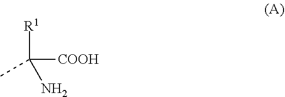
wherein R¹ is H or a C₁-C₃ alkyl group optionally substituted with one or two groups selected from —OH, —OMe, —SMe, —COOH, and —CONH₂, and the dashed bond indicates where said group of Formula (A) is connected to said fused polycyclic ring system.

8-10. (canceled)

11. The carbon quantum dot of claim 1, which comprises a polycyclic aromatic or partially aromatic ring system comprising at least 10 fused 6-membered rings, wherein the polycyclic aromatic or partially aromatic ring system is fused to at least one subunit of Formula B: 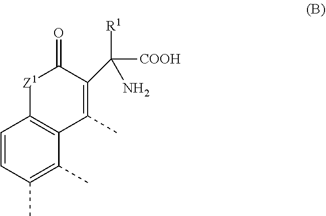
wherein:
R¹ is H or a C₁-C₃ alkyl optionally substituted with one or two groups selected from halo, —OH, —OMe, —SMe, —COOH, and —CONH₂,
Z¹ is NR² or C(R²)₂, where each R² is independently selected from H and C₁-C₃ alkyl, and
wherein the dashed bonds indicate where the Formula B is fused to the polycyclic aromatic ring system.

12-20. (canceled)

21. The carbon quantum dot of claim 1, which is formed by reacting at least two different precursors, at least one precursor comprising carboxyl and hydroxyl groups, and at least another precursor comprising a plurality of 6-member aromatic rings and at least two amino groups.

22-23. (canceled)

24. The carbon quantum dot of claim 1, which is formed by reacting at least two different precursors, at least one precursor comprising an alpha-amino carboxylic acid compound or an alpha-hydroxy carboxylic acid compound, and a second precursor comprising a C₁-C₈ alcohol.

25-26. (canceled)

27. The carbon quantum dot of claim 1, which is configured to selectively enter a cell that expresses a large neutral amino acid transporter (LAT1), or a subunit thereof.

28-29. (canceled)

30. The carbon quantum dot of claim 1, wherein:
the cell has a ratio between LAT1 gene expression level and expression level of another gene, e.g., a house keeping gene such as GAPDH, of at least 0.5; or
the LAT1 preferentially internalizes a branched-chain amino acid and/or an aromatic amino acid.

31-35. (canceled)

36. The carbon quantum dot of claim 27, wherein:
the LAT1 is preferentially or highly expressed in a targeted organ relative to other tissue(s) of a subject, e.g., a mammal; or
the LAT1 is preferentially or highly expressed in tumor or cancer cells relative to other tissue(s) or cells of a subject, e.g., a mammal.

37-38. (canceled)

39. The carbon quantum dot of claim 1, which:
has a size or diameter ranging from about 0.2 nm to about 10 nm;
has an excitation wavelength ranging from about 300 nm to about 900 nm, e.g., about 600 nm;
has an emission wavelength ranging from about 400 nm to about 1,000 nm, e.g., about 700 nm;
is configured for photoacoustic (PA) imaging upon radiation;
emits near infrared (NIR) fluorescence (FL) and is configured for photoacoustic (PA) imaging upon radiation; or
which is configured for deep tissue, tumor or cancer imaging.

40-48. (canceled)

49. The carbon quantum dot of claim 1, which:
is configured to selectively enter a tumor or cancer cell; or
is configured to selectively enter nucleus of a tumor or cancer cell.

50-54. (canceled)

55. A method for preparing a polycyclic compound or a particle, which method comprises solvothermal synthesis using at least two different precursors, wherein:
1) at least one precursor comprises an alpha-amino carboxylic acid compound or an alpha-hydroxy carboxylic acid compound, and at least one other precursor that comprises a plurality of 6-membered aromatic rings; or
2) at least one precursor comprising phenylalanine or a phenylalanine analog, e.g. an analog of phenylalanine having a substituent on the phenyl ring that is selected from halo, hydroxy, methoxy, methyl, and CF₃, and a C₁-C₈ alcohol.

56-68. (canceled)

69. The carbon quantum dot of claim 1, which further comprises a releasable cargo, e.g. the carbon quantum dot is covalently or non-covalently linked with a releasable cargo.

70-80. (canceled)

81. A method for sensing, marking or imaging a target cell, tissue or organ in a subject, which method comprises:
a) administering, to a subject in need, an effective amount of a carbon quantum dot of claim 1; and
b) assessing said carbon quantum dot for sensing, marking or imaging a target cell, tissue or organ in said subject.

82-83. (canceled)

84. The method of claim 81, which:
is used for sensing, marking or imaging a target cell, tissue or organ in a subject;
is used for sensing, marking or imaging an abnormal or diseased cell, tissue or organ in a subject;
is used for sensing, marking or imaging a tumor or cancer cell, tissue or organ in a subject.

85-86. (canceled)

87. The method of claim 84, which:
is used for diagnosis, prognosis, stratification, risk assessment, or treatment monitoring of a disease or disorder in a subject; or
is used for assisting or guiding therapy or treatment of a disease or disorder in a subject.

88. (canceled)

89. The method of claim 87, wherein the therapy or treatment comprises a procedure or surgery on a subject; or which is used for assisting or guiding a procedure or surgery on a tumor or cancer in a subject.

90-91. (canceled)

92. A pharmaceutical composition, which comprises an effective amount of a carbon quantum dot of claim 1, and optionally further comprising a therapeutic agent for treating a cancer, such as topotecan hydrochloride (TPTC), doxorubicin (DOX), or hydroxycamptothecin (HCPT), or a kinase inhibitor, and wherein the therapeutic agent is optionally releasably linked to the carbon quantum dot.

93. A method for treating or preventing a disease or condition in a subject, which comprises administering to a subject in need thereof an effective amount of a carbon quantum dot of claim 1.

94. The method of claim 93, which is used for treating or preventing a tumor or cancer in a subject, wherein the carbon quantum dot optionally comprises a therapeutic agent for treating a cancer, such as topotecan hydrochloride (TPTC), doxorubicin (DOX), or hydroxycamptothecin (HCPT), or a kinase inhibitor, and wherein the therapeutic agent is optionally releasably linked to the carbon quantum dot.

95-102. (canceled)

103. The carbon quantum dot of claim 1 that is attached to or immobilized on a solid surface or support.

104-109. (canceled)

110. A method for detecting and/or isolating a tumor or cancer cell from a sample, which method comprises contacting a sample containing or suspected of containing a tumor or cancer cell with a carbon quantum dot of claim 103, under suitable conditions, to allow binding of said tumor or cancer cell, if present in said sample, to said carbon quantum dot attached to or immobilized on said solid surface or support.

111. (canceled)

112. The method of claim 81, which is used to detect and/or isolate a circulating tumor or cancer cell from a biological sample, e.g., a blood or urine sample.

113. The method of claim 103, which is used to detect and/or isolate a circulating tumor or cancer cell from a biological sample, e.g., a blood or urine sample.
